(19) United States
(12) Patent Application Publication
(10) Pub. No.: US 2025/0002493 A1
(43) Pub. Date: Jan. 2, 2025

(54) PYRIDONE COMPOUND AND PREPARATION METHOD THEREFOR, PHARMACEUTICAL COMPOSITION AND USE

(71) Applicant: NANJING CHINA-AUSTRALIA INSTITUTE OF TRANSLATIONAL MEDICINE CO., LTD., Nanjing (CN)

(72) Inventors: Ming ZHANG, Nanjing (CN); Jing Zhan, Nanjing (CN); Tingting Liu, Nanjing (CN); Guangyi Shi, Nanjing (CN); Xiuqin Zheng, Nanjing (CN); Panhai Chen, Nanjing (CN)

(73) Assignee: NANJING CHINA-AUSTRALIA INSTITUTE OF TRANSLATIONAL MEDICINE CO., LTD., Nanjing (CN)

(21) Appl. No.: 18/817,895

(22) Filed: Aug. 28, 2024

Related U.S. Application Data
(63) Continuation-in-part of application No. PCT/CN2023/115159, filed on Aug. 28, 2023.

(30) Foreign Application Priority Data
Nov. 7, 2022 (CN) 202211386065.5

(51) Int. Cl. C07D 471/04; A61K 31/437; A61P 35/00

(52) U.S. Cl. CPC C07D 471/04; A61K 31/437; A61P 35/00

(57) ABSTRACT

A pyridone compound and a preparation method therefor, a pharmaceutical composition and a use. The structure of the pyridone compound is as shown in formula (I), and the pyridone compound comprises an isomer, a pharmaceutically acceptable salt, or a mixture thereof. The pyridone compound has a highly effective inhibitory effect on cIAP1, cIAP2, XIAP and various tumor cells, and can be used to prepare anti-tumor drugs that can exert pharmacological effects at both the molecular level and the cellular level.

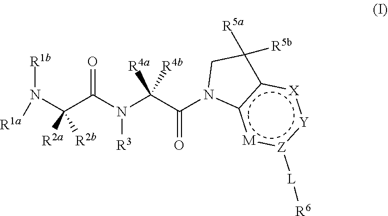

### TECHNICAL FIELD

The present invention relates to pyridone compounds and a preparation methods therefor, pharmaceutical compositions and use. In particular, the present invention relates to pyridone compounds with anti-tumor activity.

### BACKGROUND

Malignant tumors, as the primary cause of human health issues, present a formidable threat to human life and well-being. According to the latest data on cancer incidence and mortality released by the International Agency for Research on Cancer (IARC) of the World Health Organization, there were a total of 19.29 million new cancer cases and 9.96 million cancer related deaths globally in 2020. Among them, China accounted for 4.57 million new cancer cases and 3 million cancer-related deaths, representing approximately 23.7% of the global incidence and 30% of global mortality rates, thereby securing its position as the leading country in both categories. Therefore, the prevention and treatment of cancer present significant challenges, necessitating urgent promotion of research and development in innovative anti-cancer drugs to alleviate the burden of this disease.

One of the hallmarks of cancer is the resistance of cancer cells to apoptotic signals, leading to impaired programmed cell death. The extensively investigated mechanisms underlying cell death encompass apoptosis, necroptosis, autophagy, ferroptosis, pyroptosis, necrosis, etc. (Galluzzi et al., Cell Death Differ. 2018, 25: 486-541). The high expression of anti-cell death protein factors enables cancer cells to evade apoptosis and necroptosis, thereby facilitating their malignant proliferation and promoting cellular variation (Fuchs et al., Nat. Rev. Mol. Cell Biol. 2015, 16: 329-344.). The process of apoptosis, which occurs naturally in programmed cell death, is stimulated and regulated by two types of signals: mitochondria-dependent endogenous apoptotic signals and exogenous apoptotic signals that rely on cell surface death receptors (Salvesen et al, Nat. Rev. Mol. Cell Biol. 2002, 3:40). Therefore, reactivating the apoptotic potential of cancer cells through inhibition of a key negative regulator in the cell death pathway represents an attractive strategy for cancer treatment.

Mitochondrial apoptosis is a primary mechanism for removing damaged cells during normal organism development, and it is induced by various apoptotic signals, including DNA damage, oxidative damage, loss of cell adhesion, and steroid hormones. (Li et al., Cell. 1998, 94: 491-501, Luo et al., Cell. 1998, 94: 481-490). When stimulated by upstream apoptotic signals, the integrity of the mitochondrial outer membrane is compromised, leading to mitochondrial outer membrane permeabilization (MOMP), the release of cytochrome c into the cytoplasm, and the activation of a series of apoptosis-effector molecules called caspases (Stantucci et al., Int. J. Biol. Macromol. 2019, 136: 1237-1246; Rodriguez et al., Genes Dev. 1999. 13: 3179-3184; Thornberry et al., Science. 1998, 281: 1312-1316). Bcl-2 family proteins play important regulatory functions as pro-apoptotic and anti-apoptotic molecules during mitochondrial apoptosis (Adams et al., Science. 1998, 281, 1322-1326; Strasser et al., Annu. Rev. Biochem. 2000, 69, 217-245). The Bcl-2 family includes two types of members, anti-apoptotic proteins such as Bcl-2, Bcl-xl and Mcl-1, and pro-apoptotic proteins such as Bax, Bak and Bid (Cory et al., Oncogene. 2003, 22: 8590-8607; Maji et al., Adv. Cancer Res. 2018, 137: 37-75). Up-regulation of anti-apoptotic molecules Bcl-2 and Bcl-xl has been found in a variety of tumors, including breast cancer, prostate cancer, glioblastoma, leukemia and lymphoma (Delbridge et al., Nat. Rev. Cancer. 2016, 16: 99-109; Kelly et al., Cell Death Differ. 2011, 18: 1414-1424; Placzek et al., 2010; Youle et al., Nat. Rev. Mol. Cell Biol. 2008, 9: 47-59). Several small molecule drugs targeting the anti-apoptotic molecules Bcl-2 and Bcl-xl are in clinical trials. Among these, Venetoclax, the first Bcl-2 inhibitor approved by the FDA in 2016, is used for treating recurrent or refractory chronic lymphocytic leukemia (CLL).

The inhibitor of apoptosis proteins (IAPs) regulate gene expression through ubiquitination and play an important role in both mitochondrial apoptosis and death receptor mediated apoptosis. IAP family proteins contain 1 to 3 baculovirus IAP repeats (BIRs) that serve as protein-protein interaction domains (Takahashi et al., J. Biol. Chem. 1998, 273: 7787-7790). The IAP family consists of eight members: cIAP1, cIAP2, XIAP, ML-IAP, NAIP, survivin, Bruce and ILP-2. Among these, cIAP1, cIAP2 and XIAP have an anti-apoptotic function (De Almagro et al., Exp. Oncol. 2012, 34:200-211; Vaux et al., Nat. Rev. Mol. Cell Biol. 2005, 6: 287; Obexer et al., Front. Oncol. 2014, 4: 197). XIAP contains three BIR domains, which mediate direct binding to caspases 3, 7, and 9 to inhibit their enzymatic activity. In contrast, cIAP1 and cIAP2 primarily exert their anti-apoptotic effects in TNFR mediated extrinsic apoptotic pathways (McComb et al., Sci. Adv. 2019, 5: eaau94332019; Vince et al., Cell, 2017, 131: 682-693; Silke et al., EMBO J. 2001, 20: 3114-3123; Bertrand et al., Mol. Cell 2008, 30: 689-700; Mahoney et al., Proc. Natl. Acad. Sci. USA 2008, 105: 11778-11783).

It has been reported that elevated levels of IAP proteins are closely associated with the occurrence of a variety of cancers (Mohamed et al., Apoptosis. 2017, 2: 1487-1509). XIAP is overexpressed in leukemia, breast cancer, lung cancer, colorectal cancer, melanoma, ovarian cancer, prostate cancer, bladder cancer, kidney cancer and thyroid cancer (Dubrez et al., Onco. Ther. 2013, 9: 1285-1304; Krajewski et al., Clin. Cancer Res. 2003, 9: 4914-4925). cIAP1 and cIAP2 are highly expressed in the samples from CLL and acute B lymphoblastic leukemia patients (Akyurek et al., Cancer. 2006, 107, 1844-1851; Munzert et al., Blood. 2002, 100: 3749-3756). The expression level of cIAP1 is significantly increased in colorectal cancer, bladder cancer and cervical B-cell CLL (Dubrez et al., Onco. Ther. 2013, 9:1285-1304; Tamm et al., Clin. Cancer Res. 2000, 9: 1796-1803). cIAP2 is overexpressed in mucosa-associated lymphoid tissue lymphoma (Gyrd-Hansen et al., Nat. Rev. Cancer. 2010, 10: 561-574). Moreover, the overexpression of IAP proteins may play a crucial role in cancer cells resistance to current tumor therapies, as they can inhibit programmed cell death induced by chemotherapy drugs, radiation, and immunotherapy. For instance, XIAP hinders Apo2 ligand and TNF-mediated apoptosis in prostate cancer (McEleny et al., Prostate. 2002, 51: 133), and its overexpression is associated with cisplatin resistance in ovarian cancer (Li et al., Endocrinology. 2001, 142:370). Therefore, targeting IAP protein families to induce programmed cell death in cancer cells and overcome resistance to current tumor drugs represents an efficacious therapeutic strategy.

cIAP1 and cIAP2 interact with TNF receptor associated factors (TRAFs) to prevent the formation of exogenous pro-apoptotic signaling complexes and influence cell survival through both canonical and non-canonical NF-κB pathways (Bertrand et al., Mol. Cell. 2008, 30: 689-700; Vince et al., Cell. 2007, 131:682-693; Rathore et al., Apoptosis. 2017, 22: 898-919). The binding of TNF to its receptor TNFR1 recruits and assembles a signaling complex composed of TRADD, TRAF2, RIPK1, cIAP1 and cIAP2 (Dowling et al., Nat. Commun. 2019, 10, 705; Füllsack et al., Cell Death Dis. 2019, 10, 122; Anderton et al., Cell Death Di er. 2019, 26, 877). cIAP1/2 ubiquitinates the components of the complex, including RIPK1, as well as itself, thereby activating the downstream canonical NF-κB pathway. Furthermore, cIAP1 and cIAP2 may also degrade NF-κB-inducing kinase (NIK) through ubiquitination, thereby activating the non-canoical NF-κB pathway to promote pro-survival gene expression (Vince et al., Cell. 2007, 131:682-693).

Natural antagonists of IAP, such as the second mitochondrial activator of caspase (SMAC), bind to the BIR domains of the IAP proteins, thereby preventing XIAP from binding to and inhibiting caspases 3, 7, and 9 (Silke et al., J. Cell Biol. 2002, 157, 115-124; Bratton et al., EMBO J. 2001, 20, 998-1009; Wu et al., Nature 2000, 408, 1008-1012). Simultaneously, the binding of IAP antagonists to the BIR3 domains can release the RING domains, activating E3 ligase activity of cIAP1 and cIAP2 and promoting their own ubiquitination and proteasome degradation (Dueber et al., Science 2011, 334, 376-380; Varfolomeev et al., Cell 2007, 131, 669-681; Vince et al., Cell 2007, 131, 682-693). The degradation of cIAP1 and cIAP2 induced by IAP antagonists prevents cIAP-mediated ubiquitination, thereby shifting TNFFR1 signal transduction from pro-survival to pro-apoptosis. The degradation of cIAP1 and cIAP2 leads to the formation of complex 2a, which consists of proteins such as TRADD, RIPK1, FADD, and caspase 8. Caspase 8 is activated and cleaved, subsequently activating caspase 3 and leading to apoptosis (Micheau et al., Cell 2003, 114, 181-190; Kitson et al., Nature 1996, 384, 372-375; Dickens et al., Mol. Cell 2012, 47, 291-305).

The interaction between SMAC and IAP protein requires the first four N-terminal residues of SMAC, known as AVPI (Ala-Val-Pro-Ile). In vitro and in vivo tests have shown that SMAC can promote intrinsic and extrinsic apoptotic cell death of resistant tumor cells such as melanoma, neuroblastoma or glioblastoma (Fulda et al., Nat. Med. 2002, 8, 808-815). Studies using antisense oligonucleotides to target XIAP have also demonstrated increased sensitivity of tumor cells to chemotherapy drugs (Carter et al., Leukemia 2003, 17, 2081-2089; Arnt et al., J. Biol. Chem. 2002, 277, 44236-44243). Therefore, designing small molecule drugs that mimic the N-terminal AVPI peptide fragments of SMAC can be an effective means to develop IAP antagonists, induce apoptosis of tumor cells, and achieve cancer treatment.

### SUMMARY OF THE INVENTION

Objective of the invention: The present invention aims to provide pyridone compounds with excellent antitumor activity and pharmacokinetic properties, and preparation methods therefor, pharmaceutical compositions and use.

Technical solution: in the first aspect of the present invention, the pyridone compound provided by the present invention has a structure as shown in Formula (I) and comprises its isomer, prodrug, stable isotope derivative, pharmaceutically acceptable salt or a mixture thereof:

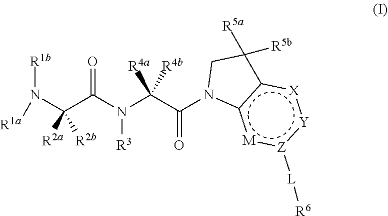

where:
R^{1a} or R^{1b} is independently selected from hydrogen or C₁˜C₆ alkyl or halogenated C₁˜C₆ alkyl, or R^{1a} or R^{1b} and the connected nitrogen atom together form 3-7-membered azacyclic alkyl;
R^{2a} or R^{2b} is independently selected from hydrogen, C₁˜C₆ alkyl or halogenated C₁˜C₆ alkyl, or R^{2a} or R^{2b} and the connected carbon atom together form C₃˜C₆ cycloalkyl or C₃˜C₆ heterocyclic alkyl, and when R^{2a} and R^{2b} are different, the carbon atom connected to R^{2a} and R^{2b} is of racemic configuration, R configuration or S configuration;
R³ is selected from hydrogen, C₁˜C₆ alkyl or C₅˜C₁₂ aryl;
R^{4a} or R^{4b} is independently selected from hydrogen, substituted C₁˜C₆ alkyl, substituted C₅˜C₁₂ aryl, substituted C₃˜C₆ cycloalkyl or C₃˜C₆ heterocyclic alkyl, the substituent is selected from hydrogen, halogen, cyano, halogenated C₁˜C₆ alkyl, C₁˜C₆ alkyl, C₁˜C₆ alkoxy, C₃˜C₆ cycloalkyl or heterocyclic alkyl, hydroxyl, amino, methylamino, dimethylamino, acetamino, carboxyl, methoxycarbonyl or nitro, there are one or more substituents, and when R^{4a} and R^{4b} are different, the connected carbon atom is of racemic configuration, R configuration or S configuration;
R^{5a} or R^{5b} is independently selected from hydrogen or C₁˜C₆ alkyl, or R^{5a} or R^{5b} and the connected carbon atom together form C₃˜C₆ cycloalkyl or C₃˜C₆ heterocyclic alkyl, and when R^{5a} and R^{5b} are different, the connected carbon atom is of racemic configuration, R configuration or S configuration;
R⁶ is selected from hydrogen, any substituted C₅˜C₁₂ aryl, any substituted C₃˜C₁₂ cycloalkyl or heterocyclic alkyl, the substituent is selected from hydrogen, halogen, cyano, halogenated C₁˜C₆ alkyl, C₁˜C₆ alkyl, C₁˜C₆ alkoxy, hydroxyl, amino, methylamino, dimethylamino, acetamino, carboxyl, methoxycarbonyl or nitro, and there are one or more substituents;
The ring A is a heteroaromatic ring, where when M is carbonyl, X and Y are CH and Z is N; when M is CH, Y is carbonyl, Z is C or N and X is CH or NR⁷;
R⁷ is selected from hydrogen, substituted C₁˜C₆ alkyl, substituted C₅˜C₁₂ aryl, substituted C₃˜C₆ cycloalkyl or C₃˜C₁₀ heterocyclic alkyl; the substituent is selected from hydrogen, halogen, cyano, halogenated C₁˜C₆ alkyl, C₁˜C₆ alkyl, C₁˜C₆ alkoxy, C₃˜C₆ cycloalkyl or C₃˜C₆ heterocyclic alkyl, hydroxyl, methoxy, amino, methylamino, dimethylamino, acetamino, carboxyl, methoxycarbonyl or nitro, and there are one or more substituents;
L is selected from oxygen, sulfur,

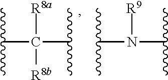

or —(CH₂)ₙ—, where the wavy lines represent points connecting the rest part of the compound;
R^{8a} or R^{8b} is independently selected from hydrogen, halogen, C₁˜C₆ alkyl or C₅˜C₁₂ aryl, and when R^{8a}, R^{8b} and R⁶ are different, the connected carbon atom is of racemic configuration, R configuration or S configuration;
R⁹ is selected from hydrogen or C₁˜C₆ alkyl or C₅˜C₁₂ aryl;
n ranges from 0 to 3.

Preferably, in the pyridone compound structure:
R^{1a} is selected from hydrogen, methyl or ethyl;
R^{1b} is selected from hydrogen;
R^{2a} or R^{2b} is independently selected from hydrogen, methyl or ethyl, or R^{2a} or R^{2b} and the connected carbon atom together form cyclopropyl, oxacyclobutyl and cyclobutyl. When R^{2a} is hydrogen, R^{2b} is methyl or ethyl, and the carbon atom connected to R^{2b} is of racemic configuration, R configuration or S configuration;
R³ is selected from hydrogen;
R^{4a} or R^{4b} is independently selected from hydrogen, cyclopropyl methyl, cyclopentyl, cyclohexyl, tetrahydropyranyl, isopropyl or tert-butyl and the carbon atom connected to R^{4a} and R^{4b} is of R or S configuration;
R^{5a} and R^{5b} are both hydrogen or methyl;
R⁶ is selected from hydrogen, substituted phenyl, substituted pyrimidyl or substituted pyridinyl, the substituent is selected from hydrogen, halogen, cyano, methoxy, hydroxyl or trifluoromethyl, and there are one or more substituents;
The ring A is a pyridine ring, where when M is carbonyl, X and Y are CH and Z is N; when M is CH, Y is carbonyl, Z is C or N and X is CH or NR⁷;
R⁷ is selected from hydrogen or methyl;
L is selected from

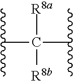

or —(CH₂)ₙ—, where the wavy lines represent points connecting the rest part of the compound;
R^{8a} or R^{8b} is independently selected from hydrogen or methyl and when R^{8a}, R^{8b} and R⁶ are different, the connected carbon atom is of racemic configuration;
n is selected from 0, 1 or 2.

More preferably, in the pyridone compound structure:
R^{1a} is methyl;
R^{2a} is hydrogen and R^{2b} is methyl;
R^{4a} is selected from cyclohexyl or tert-butyl; R^{4b} is hydrogen.

More preferably, the structure of the pyridone compound can be represented by formulae (II-1, II-2 and II-3), or tautomeric or stereochemically isomeric form thereof, a pharmaceutically acceptable salt or a solvate; where R^{1a}, R^{2a}, R^{2b}, R^{4a}, R^{4b}, R^{5a}, R^{5b}, R⁶, R⁷ and L are as defined in claims.

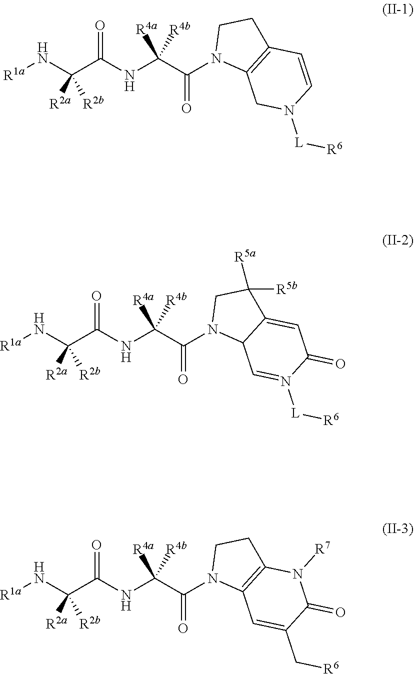

More preferably, in the structure of pyridone compound:
R⁶ is selected from

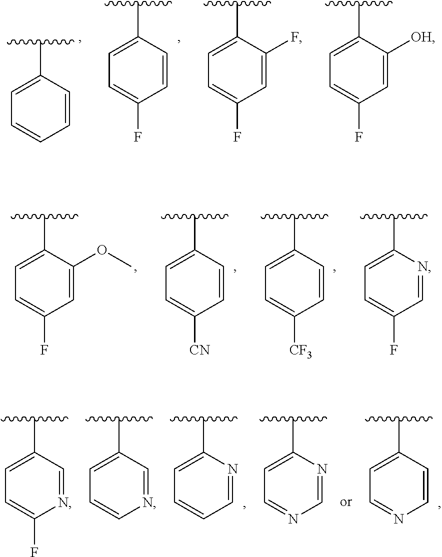

where the wavy lines represent points connecting the rest part of the compound.

More preferably, in the structure of pyridone compound:
L is selected from —(CH₂)ₙ—, where n is 1.

More specifically, the pyridone compound is selected from:

| Com- | |
|---|---|
| pound | |
| No. | Compound structure |
| I-1-1 | 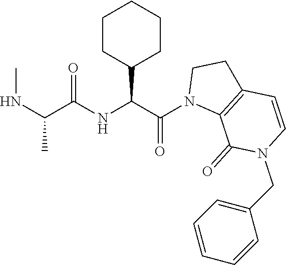 [SMILES: CN[C@@H](C)C(=O)N[C@H](C(=O)N1CCc2ccn(Cc3ccccc3)c(=O)c21)C1CCCCC1] |
| I-1-2 | 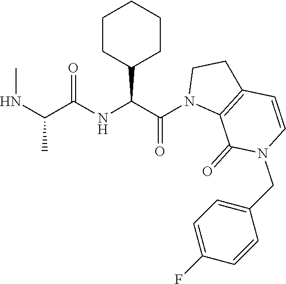 [SMILES: CN[C@@H](C)C(=O)N[C@H](C(=O)N1CCc2ccn(Cc3ccc(F)cc3)c(=O)c21)C1CCCCC1] |
| I-1-3 | 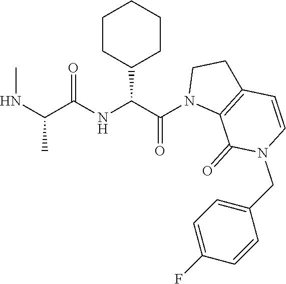 [SMILES: CN[C@@H](C)C(=O)N[C@@H](C(=O)N1CCc2ccn(Cc3ccc(F)cc3)c(=O)c21)C1CCCCC1] |
| I-1-4 | 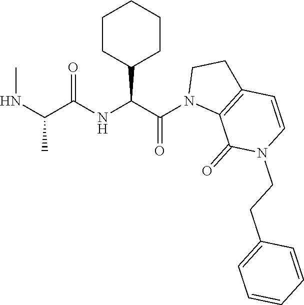 [SMILES: CN[C@@H](C)C(=O)N[C@H](C(=O)N1CCc2ccn(CCc3ccccc3)c(=O)c21)C1CCCCC1] |
| I-1-5 | 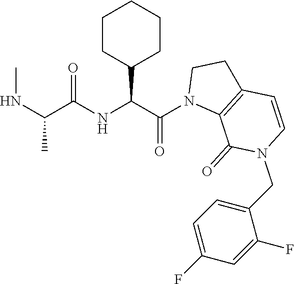 [SMILES: CN[C@@H](C)C(=O)N[C@H](C(=O)N1CCc2ccn(Cc3ccc(F)cc3F)c(=O)c21)C1CCCCC1] |
| I-1-6 | 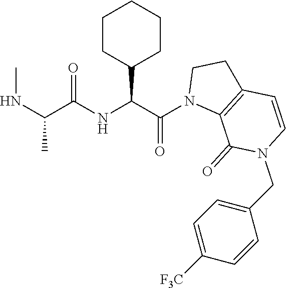 [SMILES: CN[C@@H](C)C(=O)N[C@H](C(=O)N1CCc2ccn(Cc3ccc(C(F)(F)F)cc3)c(=O)c21)C1CCCCC1] |
| I-1-7 | 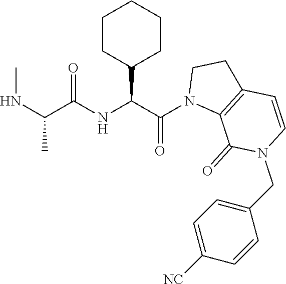 [SMILES: CN[C@@H](C)C(=O)N[C@H](C(=O)N1CCc2ccn(Cc3ccc(C#N)cc3)c(=O)c21)C1CCCCC1] |
| I-1-8 | 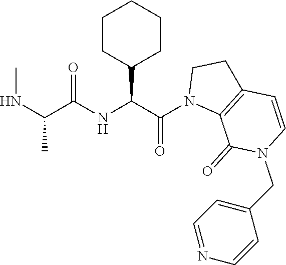 [SMILES: CN[C@@H](C)C(=O)N[C@H](C(=O)N1CCc2ccn(Cc3ccncc3)c(=O)c21)C1CCCCC1] |
| I-1-9 | 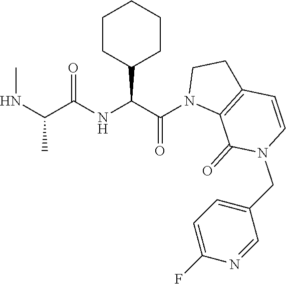 [SMILES: CN[C@@H](C)C(=O)N[C@H](C(=O)N1CCc2ccn(Cc3ccc(F)nc3)c(=O)c21)C1CCCCC1] |
| I-1-10 | 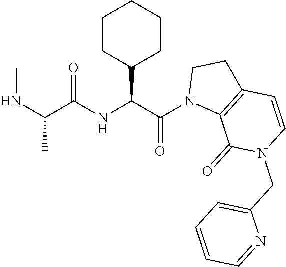 [SMILES: CN[C@@H](C)C(=O)N[C@H](C(=O)N1CCc2ccn(Cc3ccccn3)c(=O)c21)C1CCCCC1] |
| I-1-11 | 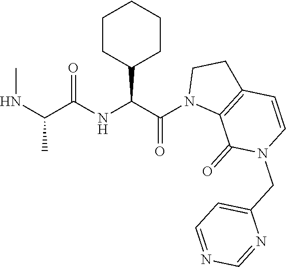 [SMILES: CN[C@@H](C)C(=O)N[C@H](C(=O)N1CCc2ccn(Cc3ccncn3)c(=O)c21)C1CCCCC1] |
| I-1-12 | 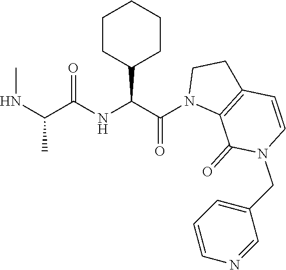 [SMILES: CN[C@@H](C)C(=O)N[C@H](C(=O)N1CCc2ccn(Cc3cccnc3)c(=O)c21)C1CCCCC1] |
| I-1-13 | 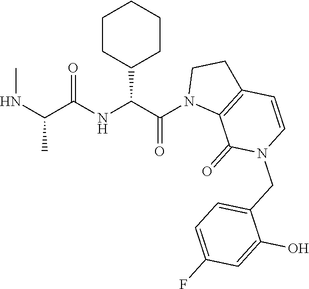 [SMILES: CN[C@@H](C)C(=O)N[C@@H](C(=O)N1CCc2ccn(Cc3ccc(F)cc3O)c(=O)c21)C1CCCCC1] |
| I-1-14 | 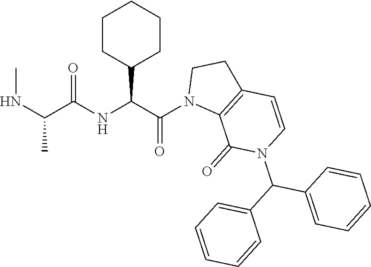 [SMILES: CN[C@@H](C)C(=O)N[C@H](C(=O)N1CCc2ccn(C(c3ccccc3)c3ccccc3)c(=O)c21)C1CCCCC1] |
| I-2-1 | 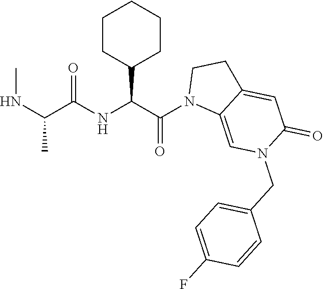 [SMILES: CN[C@@H](C)C(=O)N[C@H](C(=O)N1CCc2cc(=O)n(Cc3ccc(F)cc3)cc21)C1CCCCC1] |
| I-2-2 | 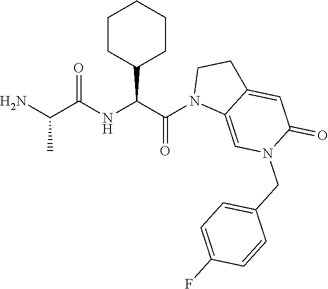 [SMILES: C[C@H](N)C(=O)N[C@H](C(=O)N1CCc2cc(=O)n(Cc3ccc(F)cc3)cc21)C1CCCCC1] |
| I-2-3 | 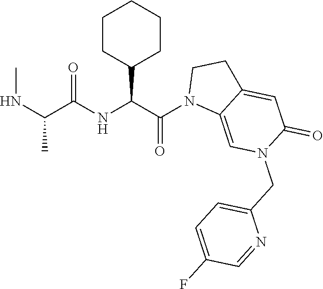 [SMILES: CN[C@@H](C)C(=O)N[C@H](C(=O)N1CCc2cc(=O)n(Cc3ccc(F)cn3)cc21)C1CCCCC1] |
| I-2-4 | 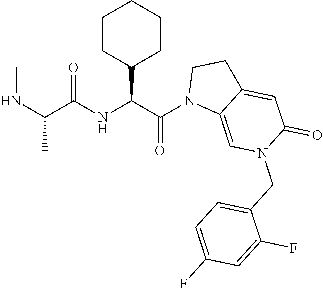 [SMILES: CN[C@@H](C)C(=O)N[C@H](C(=O)N1CCc2cc(=O)n(Cc3ccc(F)cc3F)cc21)C1CCCCC1] |
| I-2-5 | 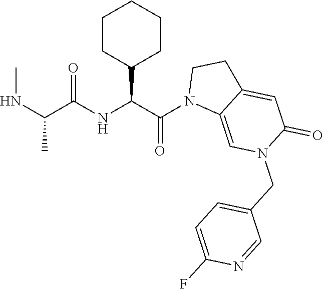 [SMILES: CN[C@@H](C)C(=O)N[C@H](C(=O)N1CCc2cc(=O)n(Cc3ccc(F)nc3)cc21)C1CCCCC1] |
| I-2-6 | 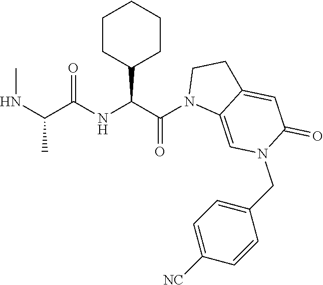 [SMILES: CN[C@@H](C)C(=O)N[C@H](C(=O)N1CCc2cc(=O)n(Cc3ccc(C#N)cc3)cc21)C1CCCCC1] |
| I-2-7 | 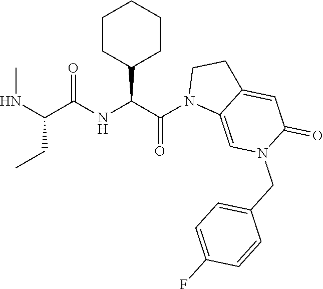 [SMILES: CC[C@H](NC)C(=O)N[C@H](C(=O)N1CCc2cc(=O)n(Cc3ccc(F)cc3)cc21)C1CCCCC1] |
| I-2-8 | 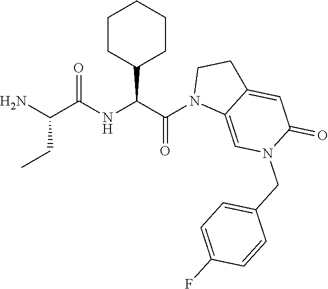 [SMILES: CC[C@H](N)C(=O)N[C@H](C(=O)N1CCc2cc(=O)n(Cc3ccc(F)cc3)cc21)C1CCCCC1] |
| I-2-9 | 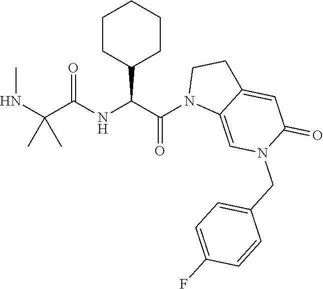 [SMILES: CNC(C)(C)C(=O)N[C@H](C(=O)N1CCc2cc(=O)n(Cc3ccc(F)cc3)cc21)C1CCCCC1] |
| I-2-10 | 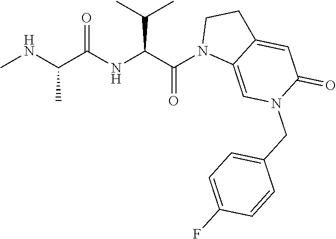 [SMILES: CN[C@@H](C)C(=O)N[C@H](C(=O)N1CCc2cc(=O)n(Cc3ccc(F)cc3)cc21)C(C)C] |
| I-2-11 | 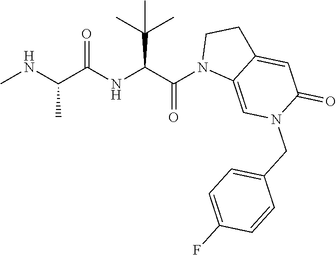 [SMILES: CN[C@@H](C)C(=O)N[C@H](C(=O)N1CCc2cc(=O)n(Cc3ccc(F)cc3)cc21)C(C)(C)C] |
| I-2-12 | 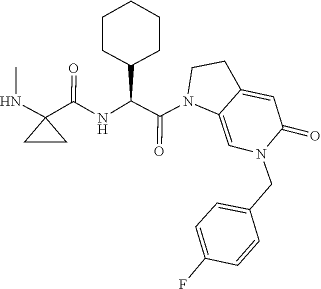 [SMILES: CNC1(C(=O)N[C@H](C(=O)N2CCc3cc(=O)n(Cc4ccc(F)cc4)cc32)C2CCCCC2)CC1] |
| I-2-13 | 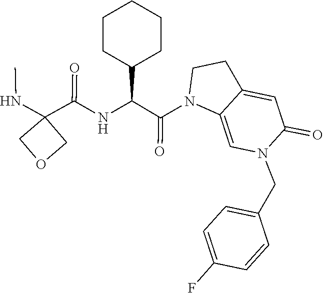 [SMILES: CNC1(C(=O)N[C@H](C(=O)N2CCc3cc(=O)n(Cc4ccc(F)cc4)cc32)C2CCCCC2)COC1] |
| I-2-14 | 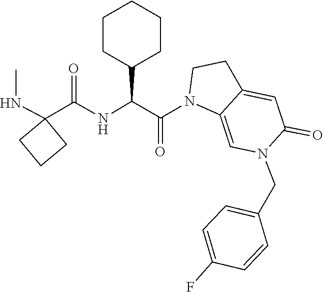 [SMILES: CNC1(C(=O)N[C@H](C(=O)N2CCc3cc(=O)n(Cc4ccc(F)cc4)cc32)C2CCCCC2)CCC1] |
| I-2-15 | 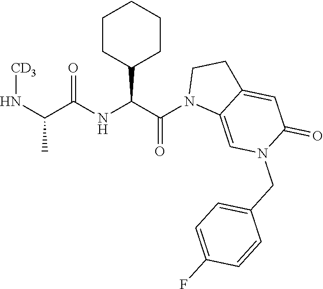 [SMILES: [2H]C([2H])([2H])N[C@@H](C)C(=O)N[C@H](C(=O)N1CCc2cc(=O)n(Cc3ccc(F)cc3)cc21)C1CCCCC1] |
| I-2-16 | 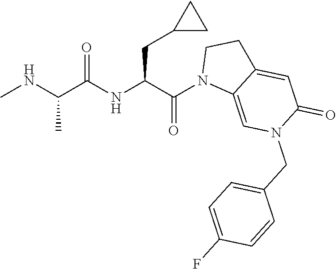 [SMILES: CN[C@@H](C)C(=O)N[C@@H](CC1CC1)C(=O)N1CCc2cc(=O)n(Cc3ccc(F)cc3)cc21] |
| I-2-17 | 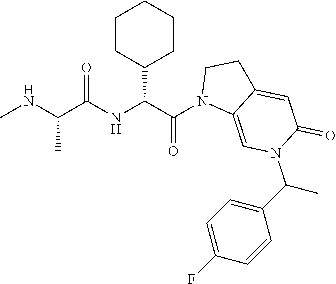 [SMILES: CN[C@@H](C)C(=O)N[C@@H](C(=O)N1CCc2cc(=O)n(C(C)c3ccc(F)cc3)cc21)C1CCCCC1] |
| I-2-18 | 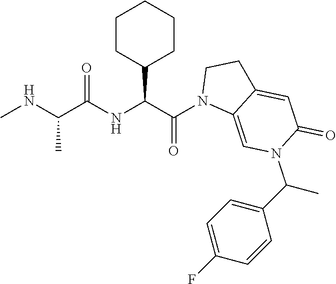 [SMILES: CN[C@@H](C)C(=O)N[C@H](C(=O)N1CCc2cc(=O)n(C(C)c3ccc(F)cc3)cc21)C1CCCCC1] |
| I-2-19 | 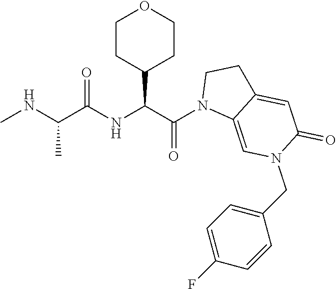 [SMILES: CN[C@@H](C)C(=O)N[C@H](C(=O)N1CCc2cc(=O)n(Cc3ccc(F)cc3)cc21)C1CCOCC1] |
| I-2-20 | 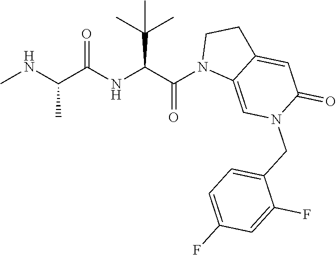 [SMILES: CN[C@@H](C)C(=O)N[C@H](C(=O)N1CCc2cc(=O)n(Cc3ccc(F)cc3F)cc21)C(C)(C)C] |
| I-2-21 | 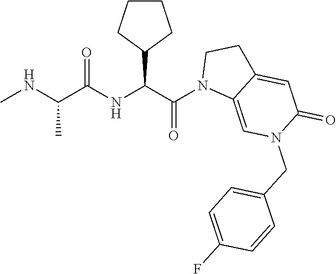 [SMILES: CN[C@@H](C)C(=O)N[C@H](C(=O)N1CCc2cc(=O)n(Cc3ccc(F)cc3)cc21)C1CCCC1] |
| I-2-22 | 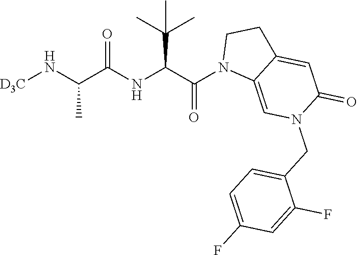 [SMILES: [2H]C([2H])([2H])N[C@@H](C)C(=O)N[C@H](C(=O)N1CCc2cc(=O)n(Cc3ccc(F)cc3F)cc21)C(C)(C)C] |
| I-2-23 | 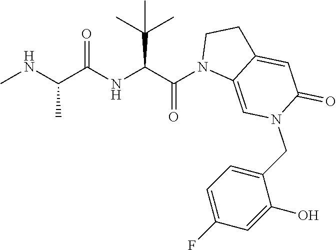 [SMILES: CN[C@@H](C)C(=O)N[C@H](C(=O)N1CCc2cc(=O)n(Cc3ccc(F)cc3O)cc21)C(C)(C)C] |
| I-2-24 | 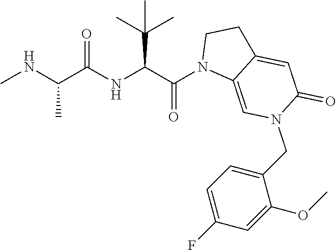 [SMILES: CN[C@@H](C)C(=O)N[C@H](C(=O)N1CCc2cc(=O)n(Cc3ccc(F)cc3OC)cc21)C(C)(C)C] |
| I-2-25 | 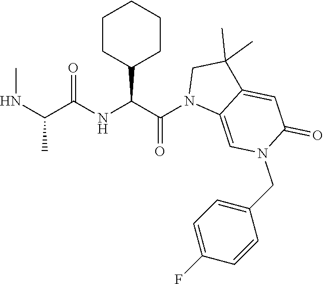 [SMILES: CN[C@@H](C)C(=O)N[C@H](C(=O)N1CC(C)(C)c2cc(=O)n(Cc3ccc(F)cc3)cc21)C1CCCCC1] |
| I-2-26 | 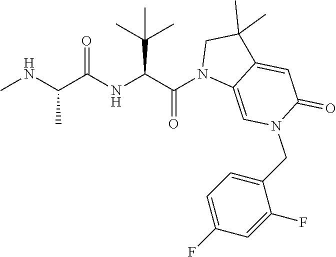 [SMILES: CN[C@@H](C)C(=O)N[C@H](C(=O)N1CC(C)(C)c2cc(=O)n(Cc3ccc(F)cc3F)cc21)C(C)(C)C] |
| I-2-27 | 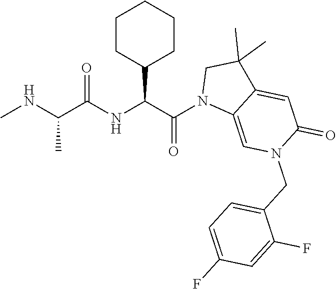 [SMILES: CN[C@@H](C)C(=O)N[C@H](C(=O)N1CC(C)(C)c2cc(=O)n(Cc3ccc(F)cc3F)cc21)C1CCCCC1] |
| I-2-28 | 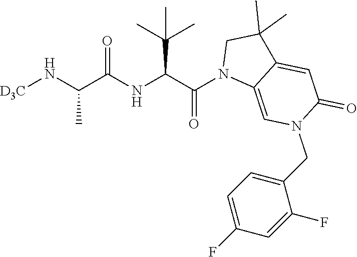 [SMILES: [2H]C([2H])([2H])N[C@@H](C)C(=O)N[C@H](C(=O)N1CC(C)(C)c2cc(=O)n(Cc3ccc(F)cc3F)cc21)C(C)(C)C] |
| I-2-29 | 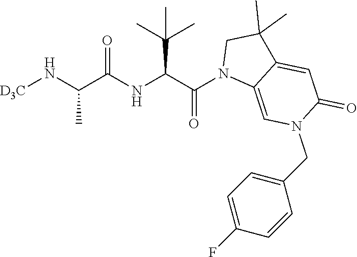 [SMILES: [2H]C([2H])([2H])N[C@@H](C)C(=O)N[C@H](C(=O)N1CC(C)(C)c2cc(=O)n(Cc3ccc(F)cc3)cc21)C(C)(C)C] |
| I-2-30 | 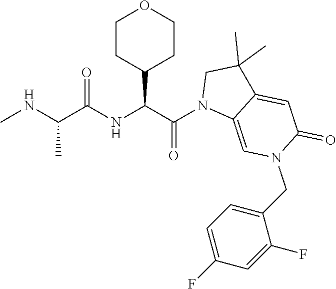 [SMILES: CN[C@@H](C)C(=O)N[C@H](C(=O)N1CC(C)(C)c2cc(=O)n(Cc3ccc(F)cc3F)cc21)C1CCOCC1] |
| I-2-31 | 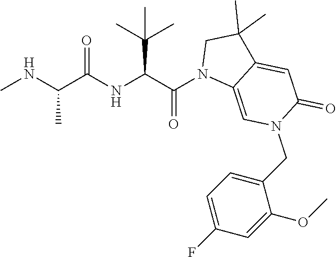 [SMILES: CN[C@@H](C)C(=O)N[C@H](C(=O)N1CC(C)(C)c2cc(=O)n(Cc3ccc(F)cc3OC)cc21)C(C)(C)C] |
| I-2-32 | 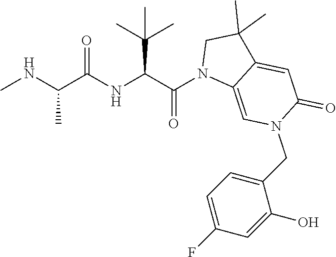 [SMILES: CN[C@@H](C)C(=O)N[C@H](C(=O)N1CC(C)(C)c2cc(=O)n(Cc3ccc(F)cc3O)cc21)C(C)(C)C] |
| I-2-33 | 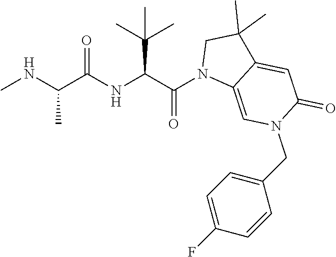 [SMILES: CN[C@@H](C)C(=O)N[C@H](C(=O)N1CC(C)(C)c2cc(=O)n(Cc3ccc(F)cc3)cc21)C(C)(C)C] |
| I-2-34 | 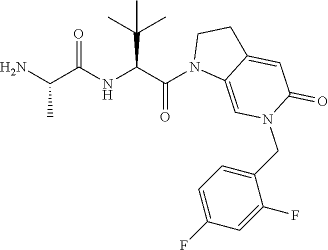 [SMILES: C[C@H](N)C(=O)N[C@H](C(=O)N1CCc2cc(=O)n(Cc3ccc(F)cc3F)cc21)C(C)(C)C] |
| I-2-35 | 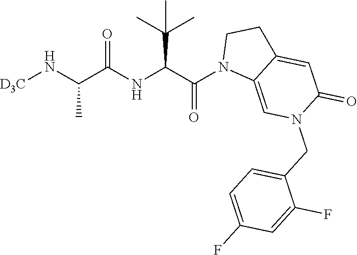 [SMILES: [2H]C([2H])([2H])N[C@@H](C)C(=O)N[C@H](C(=O)N1CCc2cc(=O)n(Cc3ccc(F)cc3F)cc21)C(C)(C)C] |
| I-3-1 | 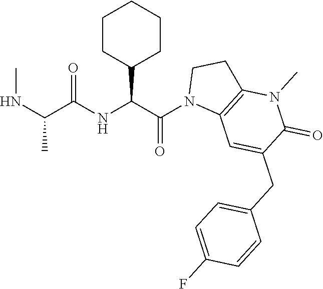 [SMILES: CN[C@@H](C)C(=O)N[C@H](C(=O)N1CCc2c1cc(Cc1ccc(F)cc1)c(=O)n2C)C1CCCCC1] |
| I-3-2 | 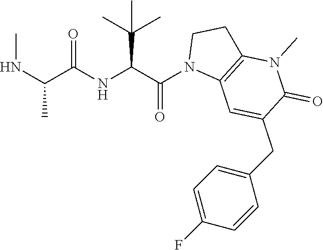 [SMILES: CN[C@@H](C)C(=O)N[C@H](C(=O)N1CCc2c1cc(Cc1ccc(F)cc1)c(=O)n2C)C(C)(C)C] |
| I-3-3 | 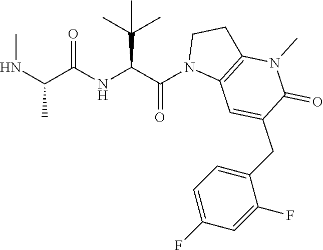 [SMILES: CN[C@@H](C)C(=O)N[C@H](C(=O)N1CCc2c1cc(Cc1ccc(F)cc1F)c(=O)n2C)C(C)(C)C] |
| I-3-4 | 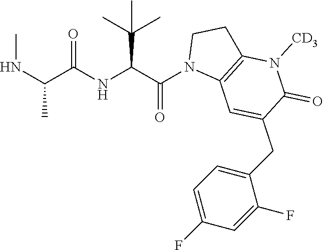 [SMILES: [2H]C([2H])([2H])n1c2c(cc(Cc3ccc(F)cc3F)c1=O)N(C(=O)[C@@H](NC(=O)[C@H](C)NC)C(C)(C)C)CC2] |
| I-3-5 | 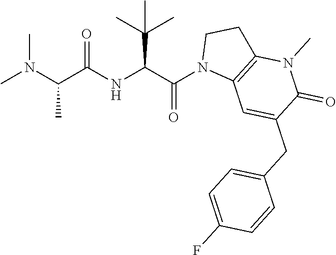 [SMILES: C[C@@H](C(=O)N[C@H](C(=O)N1CCc2c1cc(Cc1ccc(F)cc1)c(=O)n2C)C(C)(C)C)N(C)C] |
| I-3-6 | 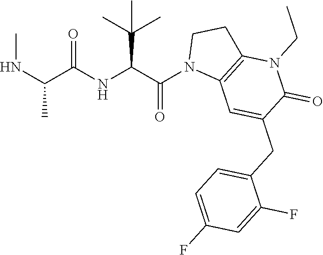 [SMILES: CCn1c2c(cc(Cc3ccc(F)cc3F)c1=O)N(C(=O)[C@@H](NC(=O)[C@H](C)NC)C(C)(C)C)CC2] |
| I-3-7 | 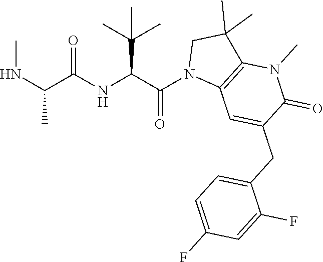 [SMILES: CN[C@@H](C)C(=O)N[C@H](C(=O)N1CC(C)(C)c2c1cc(Cc1ccc(F)cc1F)c(=O)n2C)C(C)(C)C] |
| I-3-8 | 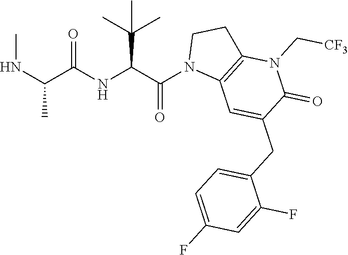 [SMILES: CN[C@@H](C)C(=O)N[C@H](C(=O)N1CCc2c1cc(Cc1ccc(F)cc1F)c(=O)n2CC(F)(F)F)C(C)(C)C] |
| I-3-9 | 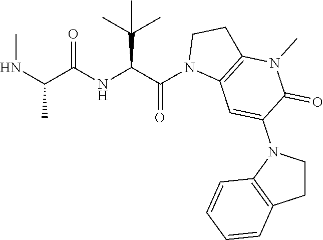 [SMILES: CN[C@@H](C)C(=O)N[C@H](C(=O)N1CCc2c1cc(N1CCc3ccccc31)c(=O)n2C)C(C)(C)C] |
| I-4-1 | 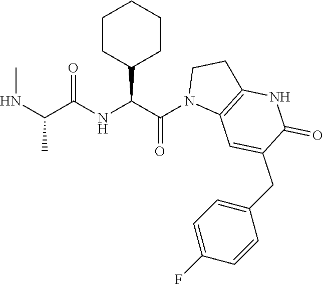 [SMILES: CN[C@@H](C)C(=O)N[C@H](C(=O)N1CCc2[nH]c(=O)c(Cc3ccc(F)cc3)cc21)C1CCCCC1] |
| I-4-2 | 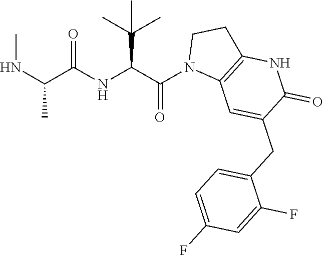 [SMILES: CN[C@@H](C)C(=O)N[C@H](C(=O)N1CCc2[nH]c(=O)c(Cc3ccc(F)cc3F)cc21)C(C)(C)C] |
| 1-4-3 | 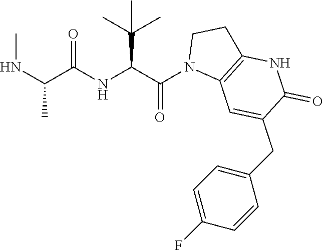 [SMILES: CN[C@@H](C)C(=O)N[C@H](C(=O)N1CCc2[nH]c(=O)c(Cc3ccc(F)cc3)cc21)C(C)(C)C] |
| I-4-4 | 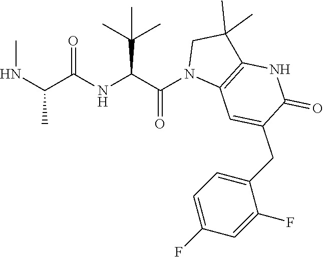 [SMILES: CN[C@@H](C)C(=O)N[C@H](C(=O)N1CC(C)(C)c2[nH]c(=O)c(Cc3ccc(F)cc3F)cc21)C(C)(C)C] |
| I-5-1 | 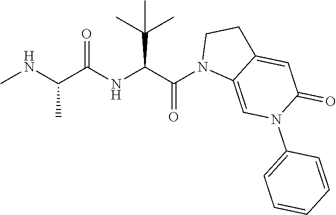 [SMILES: CN[C@@H](C)C(=O)N[C@H](C(=O)N1CCc2cc(=O)n(-c3ccccc3)cc21)C(C)(C)C] |
| I-5-2 | 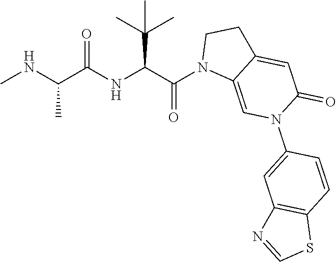 [SMILES: CN[C@@H](C)C(=O)N[C@H](C(=O)N1CCc2cc(=O)n(-c3ccc4scnc4c3)cc21)C(C)(C)C] |

The pharmaceutically acceptable salt of the above pyridone compound is a salt formed by the pyridone compound and an acid. The acid is hydrochloric acid, hydrobromic acid, sulfuric acid, phosphoric acid, carbonic acid, methanesulfonic acid, benzenesulfonic acid, p-toluenesulfonic acid, naphthalene sulfonic acid, citric acid, tartaric acid, lactic acid, pyruvic acid, acetic acid, maleic acid, succinic acid, fumaric acid, salicylic acid, phenylacetic acid, mandelic acid or ferulic acid.

Unless otherwise indicated, the term “alkyl” used herein includes branched and straight-chain saturated aliphatic hydrocarbon groups with a specific number of carbon atoms, including all isomers. Some alkyl groups can be expressed in an abbreviated form. For example, methyl can be denoted by “Me” or CH₃, ethyl can be denoted by “Et” or CH₂CH₃, propyl by “Pr” or CH₂CH₂CH₃, and butyl by “Bu” or CH₂CH₂CH₂CH₃. For example, “C₁₋₆alkyl” refer to straight or branched alkyl groups with a specific number of carbon atoms, including all isomers. The term “C₅₋₁₂ alkyl” has a similar meaning.

The term “halogen” (or “halogenated”) refers to fluorine, chlorine, bromine and iodine (or referred to as fluoro (F), chloro (Cl), bromo (Br) and iodo (I)).

The term “aryl” refers to monocyclic and polycyclic aromatic systems. In a polycyclic system, the carbon rings are condensed or connected to each other by single bonds. In general, aryl groups include phenyl, naphthyl and biphenyl.

The term “heterocycle” refers to a cyclic structure composed of carbon and non-carbon atoms, such as nitrogen, oxygen and sulfur. In general, heterocyclic groups include pyridine, quinoline, tropane, phenothiazine, benzodiazepine, furan, pyrazolone and pyrimidine.

The term “aromatic heterocycle” refers to a 5 or 6-membered single aromatic ring or a 7-12-membered double ring consisting of carbon atoms and one or more heteroatoms selected from N, O and S. Examples of aromatic heterocycle include pyridinyl, pyrryl, pyrazinyl, pyrimidyl, pyridazinyl, thiophenyl, thiazolyl, furanyl, imidazolyl, pyrazolyl, triazolyl, tetrazolyl, oxazolyl, isoxazolyl, diazolyl, thiazolyl, isothiazolyl, thiadiazolyl, benzotriazolyl, indolyl, isoindolyl, indazolyl, dihydroindazolyl, isodihydroindazolyl, quinoxalinyl, quinazolinyl, cinolinyl, chromanyl, isochromanyl, tetrahydroquinolyl, quinolyl, tetrahydroisoquinolyl, isoquinolyl, 2,3-dihydrobenzofuranyl, 2,3-dihydrobenzo-1, 4-dienyl, imidazo(2,1-b)(1,3)thiazole and benzo-1,3-m-dioxole.

The aryl in the term “substituted aryl” is as defined above. When no substituent is specified for the substituted aryl, the substituent can be selected from the following groups, including but not limited to: halogen, C₁-C₂₀alkyl, CF₃, NH₂, N(C₁-C₆ alkyl)₂, NO₂, oxo, CN, N₃, —OH, —O(C₁-C₆alkyl), C₃-C₁₀cycloalkyl, C₂-C₆alkenyl, C₂-C₆alkynyl, (C₀-C₆alkyl)S(O)₀₋₂—, aryl-S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆alkyl)CF₃, (C₀-C₆ alkyl)C(O)—, (C₀-C₆ alkyl)OC(O)—, (C₀-C₆ alkyl)₂NC(O)—(C₀-C₆ alkyl)O(C₁-C₆ alkyl)-, (C₀-C₆alkyl)C(O)₁₋₂(C₀-C₆alkyl)-, (C₀-C₆ alkyl)OC(O)NH—, aryl, aralkyl, heteroaryl, heterocyclic alkyl, halogen-aryl, halogen-aralkyl, halogen-heterocycle, halogen-heterocyclic alkyl, cyano-aryl, cyano-aralkyl, cyano-heterocycle and cyano-heterocyclic alkyl. The term “substituted phenyl” has a similar definition.

The preparation method for the pyridone compound is any one of the following methods:

(1) When R^{1b} and R³ are hydrogen and L is

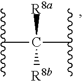

the preparation method for the compound as shown Formula (II-1) is as follows:

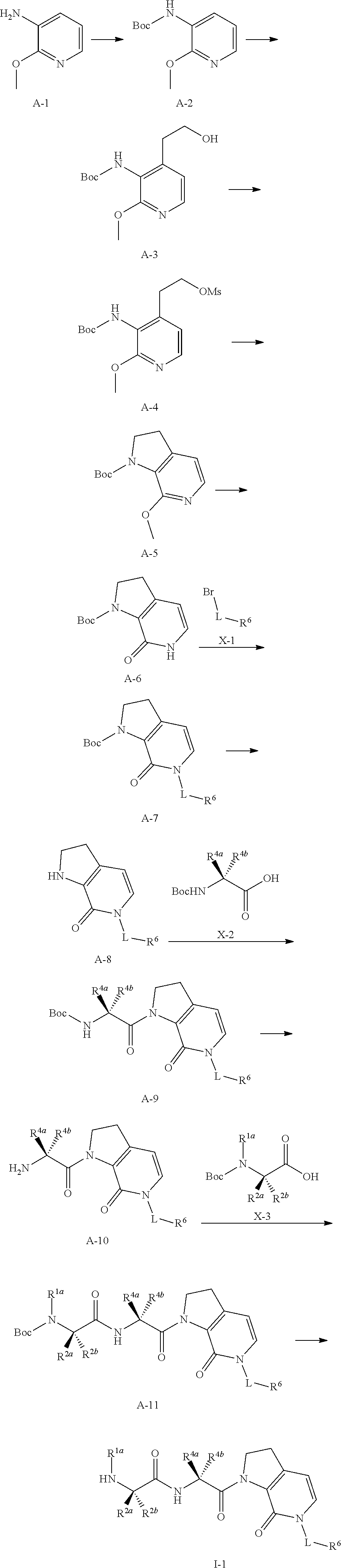

Compound A-2 is prepared from compound A-1 by dissolving both A-1 and Boc anhydride (Boc₂O) in a solvent and adding an organic alkali and an additive for amino protection reaction. The reaction solvent is N,N-dimethylformamide (DMF), tetrahydrofuran (THF), 1,4-dioxane, dichloromethane (DCM) or diethyl ether, preferably DCM; the organic alkali is triethylamine (TEA) or N,N-diisopropylethylamine (DIPEA), preferably TEA; the additive is 4-dimethylaminopyridine (DMAP).

Compound A-3 is prepared from compound A-2 by dissolving both A-2 and ethylene oxide in a dry solvent and adding an organic alkali and n-butyl lithium for reaction at −78° C. The reaction solvent is tetrahydrofuran (THF), 1,4-dioxane, dichloromethane (DCM) or diethyl ether, preferably diethyl ether; the organic alkali is triethylamine (TEA), N,N-diisopropylethylamine (DIPEA) or tetramethylenediamine (TMEDA), preferably TMEDA.

Compound A-4 is prepared from compound A-3 by dissolving both A-3 and methylsulfonyl chloride in a solvent and adding an organic alkali for hydroxyl protection reaction. The reaction solvent is N,N-dimethylformamide (DMF), tetrahydrofuran (THF), 1,4-dioxane, dichloromethane (DCM) or diethyl ether, preferably DCM; the organic alkali is triethylamine (TEA) or N,N-diisopropylethylamine (DIPEA), preferably TEA.

Compound A-5 is prepared from compound A-4 by dissolving A-4 in a solvent and adding an alkali for reaction. The reaction solvent is N,N-dimethylformamide (DMF), tetrahydrofuran (THF), 1,4-dioxane, dichloromethane (DCM) or diethyl ether, preferably tetrahydrofuran (THF); the alkali is lithium bis(trimethylsilyl)amide (LiHMDS).

Compound A-6 is prepared from compound A-5 by dissolving both A-5 and NaSCH₃ in a solvent and heating them at 100° C. for reaction. The reaction solvent is N,N-dimethylformamide (DMF) or dimethyl sulfoxide (DMSO), preferably N,N-dimethylformamide (DMF).

Compound A-7 is prepared from compound A-6 by dissolving both A-6 and X-1 in a solvent and adding an organic alkali for substitution reaction. The reaction solvent is N,N-dimethylformamide (DMF), dimethyl sulfoxide (DMSO) or 1,4-dioxane, preferably N,N-dimethylformamide (DMF); the inorganic alkali is sodium carbonate, potassium carbonate, cesium carbonate or sodium hydroxide, preferably cesium carbonate.

Compound A-8 is prepared from compound A-7 by dissolving A-7 in a solvent and adding them to an ethyl acetate solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably ethyl acetate.

Compound A-9 is prepared from compound A-8 by dissolving A-8 in a solvent, adding a condensing agent and then adding an alkali and compound X-2 for condensation reaction. The solvent is dichloromethane, tetrahydrofuran (THF), N,N-dimethylformamide (DMF), 1,4-dioxane, glycol dimethyl ether or acetonitrile, preferably tetrahydrofuran (THF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU) orbenzotriazole-1-yl-oxytripyrrolidinophosphonium hexafluorophosphate (PyBop), preferably 4-(4,6-dimethoxy-1,3,5-triazin-2-yl)-4-methyl morpholinium chloride (DMTMM); the alkali is triethylamine, sodium bicarbonate, sodium carbonate, potassium carbonate or DIPEA, preferably DIPEA.

Compound A-10 is prepared from compound A-9 by dissolving A-9 in a solvent and adding an acid for deprotection. The reaction solvent is N,N-dimethylformamide (DMF), tetrahydrofuran (THF), 1,4-dioxane, dichloromethane (DCM) or ethyl acetate, preferably dichloromethane (DCM); the acid is hydrochloric acid, formic acid or trifluoroacetic acid, preferably formic acid.

Compound A-11 is prepared from compound A-10 by dissolving A-10 in a solvent, adding a condensing agent and then adding an alkali and compound X-3 for condensation reaction. The solvent is dichloromethane, tetrahydrofuran (THF), N,N-dimethylformamide (DMF), 1,4-dioxane, glycol dimethyl ether or acetonitrile, preferably N,N-dimethylformamide (DMF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU), 1-hydroxybenzotriazole (HOBT) orbenzotriazole-1-yl-oxytripyrrolidinophosphonium hexafluorophosphate (PyBop), preferably 1-(3-dimethylaminopropyl)-3-ethyl-carbodiimid monohydrochloride (EDCI) and 1-hydroxybenzotriazole (HOBT); the alkali is triethylamine, sodium bicarbonate, sodium carbonate, potassium carbonate or DIPEA, preferably DIPEA.

Target compound I-1 is prepared from compound A-11 by dissolving A-11 in a solvent and adding an acid for deprotection. The reaction solvent is N,N-dimethylformamide (DMF), tetrahydrofuran (THF), 1,4-dioxane, dichloromethane (DCM), acetonitrile or ethyl acetate, preferably dichloromethane (DCM); the acid is hydrochloric acid, formic acid or trifluoroacetic acid, preferably formic acid.

(2) When R^{1b} and R³ are hydrogen and L is

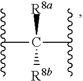

the preparation method for the compound as shown in Formula (II-2) is as follows:

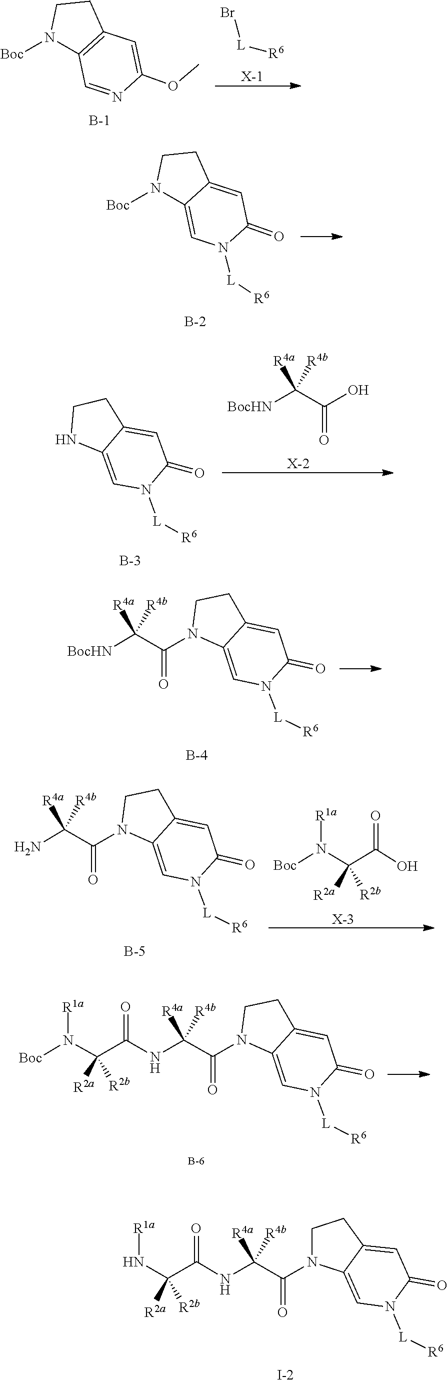

Compound B-2 is prepared from compound B-1 by dissolving both B-1 and NaI in a solvent and adding X-1 for substitution reaction. The reaction solvent is N,N-dimethylformamide (DMF), tetrahydrofuran (THF), 1,4-dioxane, dichloromethane (DCM), acetonitrile or ethyl acetate, preferably acetonitrile.

Compound B-3 is prepared from compound B-2 by dissolving B-2 in a solvent and adding them to an ethyl acetate solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably ethyl acetate.

Compound B-4 is prepared from compound B-3 by dissolving B-3 in a solvent, adding a condensing agent and then adding an alkali and compound X-2 for condensation reaction. The solvent is dichloromethane, tetrahydrofuran (THF), N,N-dimethylformamide (DMF), 1,4-dioxane, glycol dimethyl ether or acetonitrile, preferably N,N-dimethylformamide (DMF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU), 1-hydroxybenzotriazole (HOBT) orbenzotriazole-1-yl-oxytripyrrolidinophosphonium hexafluorophosphate (PyBop), preferably 2-(7-azabenzotriazol-1-yl)-N,N,N′,N′-tetramethyluronium hexafluorophosphate (HATU); the alkali is triethylamine (TEA), sodium bicarbonate, sodium carbonate, potassium carbonate or DIPEA, preferably triethylamine.

Compound B-5 is prepared from compound B-4 by dissolving B-4 in a solvent and adding an acid for deprotection. The reaction solvent is N,N-dimethylformamide (DMF), tetrahydrofuran (THF), 1,4-dioxane, dichloromethane (DCM), acetonitrile or ethyl acetate, preferably dichloromethane (DCM); the acid is hydrochloric acid, formic acid or trifluoroacetic acid, preferably formic acid.

Compound B-6 is prepared from compound B-5 by dissolving B-5 in a solvent, adding a condensing agent and then adding an alkali and compound X-3 for condensation reaction. The solvent is dichloromethane, tetrahydrofuran (THF), N,N-dimethylformamide (DMF), 1,4-dioxane, glycol dimethyl ether or acetonitrile, preferably N,N-dimethylformamide (DMF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU), 1-hydroxybenzotriazole (HOBT) orbenzotriazole-1-yl-oxytripyrrolidinophosphonium hexafluorophosphate (PyBop), preferably 1-(3-dimethylaminopropyl)-3-ethyl-carbodiimid monohydrochloride (EDCI) and 1-hydroxybenzotriazole (HOBT); the alkali is triethylamine, sodium bicarbonate, sodium carbonate, potassium carbonate or DIPEA, preferably triethylamine.

Target compound I-2 is prepared from compound B-6 by dissolving B-6 in a solvent and adding an acid for deprotection. The reaction solvent is N,N-dimethylformamide (DMF), tetrahydrofuran (THF), 1,4-dioxane, dichloromethane (DCM), acetonitrile or ethyl acetate, preferably dichloromethane (DCM); the acid is hydrochloric acid, formic acid or trifluoroacetic acid, preferably formic acid.

(3) When R^{1b} and R³ are hydrogen, R⁷ is alkyl and L is

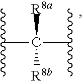

the preparation method for the compound as shown in Formula (II-3) is as follows:

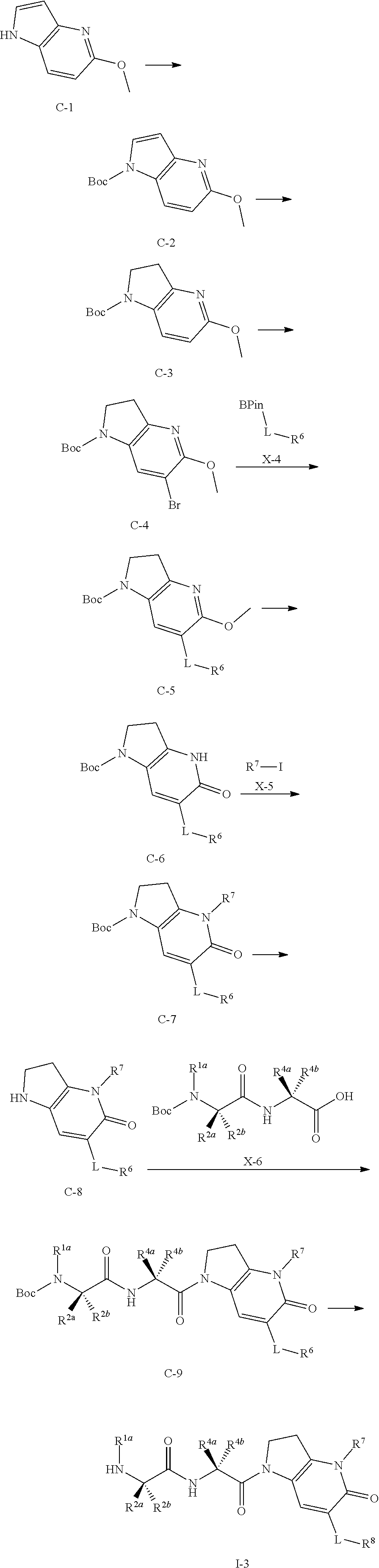

Compound C-2 is prepared from compound C-1 by dissolving both C-1 and Boc anhydride (Boc₂O) in a solvent and adding an organic alkali and an additive for amino protection reaction. The reaction solvent is N,N-dimethylformamide (DMF), tetrahydrofuran (THF), 1,4-dioxane, dichloromethane (DCM), methanol or diethyl ether, preferably DCM; the organic alkali is triethylamine (TEA) or N,N-diisopropylethylamine (DIPEA), preferably TEA; the additive is 4-dimethylaminopyridine (DMAP).

Compound C-3 is prepared from compound C-2 by dissolving C-2 in a solvent and adding Pd/C and Pd(OH)₂ in turn for catalytic reduction in a hydrogen atmosphere. The reaction solvent is N,N-dimethylformamide (DMF), tetrahydrofuran (THF), 1,4-dioxane, dichloromethane (DCM), methanol or ethanol, preferably methanol.

Compound C-4 is prepared from compound C-3 by dissolving C-3 in a solvent and adding a brominating agent for substitution reaction. The reaction solvent is N,N-dimethylformamide (DMF), tetrahydrofuran (THF), 1,4-dioxane, dichloromethane (DCM), acetonitrile or diethyl ether, preferably N,N-dimethylformamide (DMF); the brominating agent is N-bromosuccinimide (NBS), 1,3-dibromo-5,5-dimethylhydantoin (DBDMH) or bromine, preferably N-bromosuccinimide (NBS).

Compound C-5 is prepared from compound C-4 by dissolving both C-4 and X-4 in a solvent and adding a palladium catalyst and an alkali in turn for coupling reaction. The reaction solvent is N,N-dimethylformamide (DMF), tetrahydrofuran (THF), 1,4-dioxane, dimethyl sulfoxide (DMSO) or water, preferably a mixed solvent of 1,4-dioxane and water; the palladium catalyst is Pd(dppf)Cl₂, Pd₂(dba)₃ or Pd(PPh₃)₄, preferably Pd(dppf)Cl₂; the alkali is sodium carbonate, cesium carbonate, potassium phosphate, potassium carbonate or potassium acetate, preferably potassium phosphate.

Compound C-6 is prepared from compound C-5 by dissolving C-5 in a solvent and adding NaSCH₅ for reaction. The reaction solvent is N,N-dimethylformamide (DMF), tetrahydrofuran (THF), 1,4-dioxane, dichloromethane (DCM), acetonitrile or diethyl ether, preferably N,N-dimethylformamide (DMF).

Compound C-7 is prepared from compound C-6 by dissolving C-6 in a solvent and adding X-5 and an alkali through substitution reaction. The reaction solvent is N,N-dimethylformamide (DMF), tetrahydrofuran (THF), 1,4-dioxane, dichloromethane (DCM), acetonitrile or diethyl ether, preferably N,N-dimethylformamide (DMF); the alkali is sodium carbonate, cesium carbonate, potassium phosphate, potassium carbonate or potassium acetate, preferably cesium carbonate.

Compound C-8 is prepared from compound C-7 by dissolving C-7 in a solvent and adding them to an ethyl acetate solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably ethyl acetate.

Compound C-9 is prepared from compound C-8 by dissolving C-8 in a solvent, adding a condensing agent and then adding an alkali and compound X-6 for condensation reaction. The solvent is dichloromethane, tetrahydrofuran (THF), N,N-dimethylformamide (DMF), 1,4-dioxane, glycol dimethyl ether or acetonitrile, preferably tetrahydrofuran (THF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU) orbenzotriazole-1-yl-oxytripyrrolidinophosphonium hexafluorophosphate (PyBop), preferably 4-(4,6-dimethoxy-1,3,5-triazin-2-yl)-4-methyl morpholinium chloride (DMTMM); the alkali is triethylamine, sodium bicarbonate, sodium carbonate, potassium carbonate or DIPEA, preferably DIPEA.

Target compound I-3 is prepared from compound C-9 by dissolving C-9 in a solvent and adding an acid for deprotection. The reaction solvent is N,N-dimethylformamide (DMF), tetrahydrofuran (THF), 1,4-dioxane, dichloromethane (DCM), acetonitrile or ethyl acetate, preferably dichloromethane (DCM); the acid is hydrochloric acid, formic acid or trifluoroacetic acid, preferably formic acid.

(4) When R^{1b}, R³ and R⁷ are hydrogen and L is

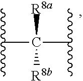

the preparation method for the compound as shown in Formula (II-3) is as follows:

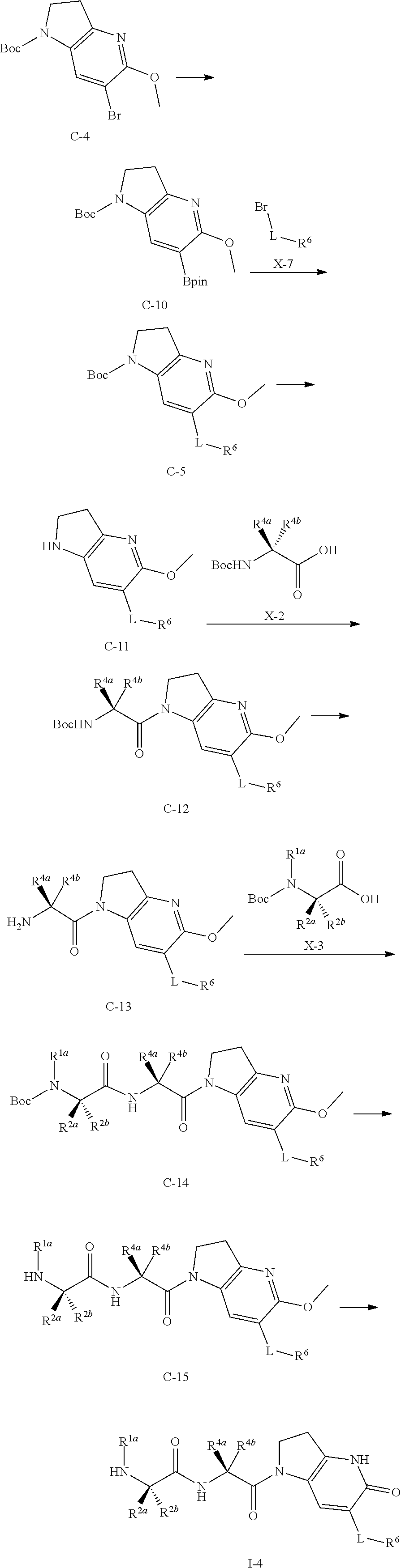

Compound C-10 is prepared from compound C-4 by dissolving both C-4 and bisdiboron in a solvent and adding a palladium catalyst and an alkali in turn for reaction. The reaction solvent is N,N-dimethylformamide (DMF), tetrahydrofuran (THF), 1,4-dioxane, toluene or acetonitrile, preferably toluene; the palladium catalyst is Pd(dppf)Cl₂, Pd₂(dba)₃ or Pd(PPh₃)₄, preferably Pd(dppf)Cl₂; the alkali is sodium carbonate, cesium carbonate, potassium phosphate, potassium carbonate or potassium acetate, preferably potassium acetate.

An alternative method for preparing compound C-5 is to dissolve both compound C-10 and X-7 in a solvent and add a palladium catalyst and an alkali in turn for reaction. The reaction solvent is N,N-dimethylformamide (DMF), tetrahydrofuran (THF), 1,4-dioxane, dimethyl sulfoxide (DMSO) or water, preferably a mixed solvent of 1,4-dioxane and water; the palladium catalyst is Pd(dppf)Cl₂, Pd₂(dba)₃ or Pd(PPh₃)₄, preferably Pd(dppf)Cl₂; the alkali is sodium carbonate, cesium carbonate, potassium phosphate, potassium carbonate or potassium acetate, preferably potassium phosphate.

Compound C-11 is prepared from compound C-5 by dissolving C-5 in a solvent and adding them to a 1,4-dioxane solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably 1,4-dioxane.

Compound C-12 is prepared from compound C-11 by dissolving C-11 in a solvent, adding a condensing agent and then adding an alkali and compound X-2 for condensation reaction. The solvent is dichloromethane, tetrahydrofuran (THF), N,N-dimethylformamide (DMF), 1,4-dioxane, glycol dimethyl ether or acetonitrile, preferably N,N-dimethylformamide (DMF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU), 1-hydroxybenzotriazole (HOBT) orbenzotriazole-1-yl-oxytripyrrolidinophosphonium hexafluorophosphate (PyBop), preferably 2-(7-azabenzotriazol-1-yl)-N,N,N′,N′-tetramethyluronium hexafluorophosphate (HATU); the alkali is triethylamine (TEA), sodium bicarbonate, sodium carbonate, potassium carbonate or DIPEA, preferably triethylamine.

Compound C-13 is prepared from compound C-12 by dissolving C-12 in a solvent and adding an acid for deprotection. The reaction solvent is N,N-dimethylformamide (DMF), tetrahydrofuran (THF), 1,4-dioxane, dichloromethane (DCM), acetonitrile or ethyl acetate, preferably dichloromethane (DCM); the acid is hydrochloric acid, formic acid or trifluoroacetic acid, preferably formic acid.

Compound C-14 is prepared from compound C-13 by dissolving C-13 in a solvent, adding a condensing agent and then adding an alkali and compound X-3 for condensation reaction. The solvent is dichloromethane, tetrahydrofuran (THF), N,N-dimethylformamide (DMF), 1,4-dioxane, glycol dimethyl ether or acetonitrile, preferably N,N-dimethylformamide (DMF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU), 1-hydroxybenzotriazole (HOBT) orbenzotriazole-1-yl-oxytripyrrolidinophosphonium hexafluorophosphate (PyBop), preferably 1-(3-dimethylaminopropyl)-3-ethyl-carbodiimid monohydrochloride (EDCI) and 1-hydroxybenzotriazole (HOBT); the alkali is triethylamine, sodium bicarbonate, sodium carbonate, potassium carbonate or DIPEA, preferably DIPEA.

Compound C-15 is prepared from compound C-14 by dissolving C-14 in a solvent and adding them to a 1,4-dioxane solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably dichloromethane (DCM).

Target compound I-4 is prepared from compound C-15 by dissolving C-15 in a solvent and adding NaSCH₅ for reaction. The reaction solvent is N,N-dimethylformamide (DMF), tetrahydrofuran (THF), 1,4-dioxane, dichloromethane (DCM), acetonitrile or diethyl ether, preferably N,N-dimethylformamide (DMF).

(5) When R^{1b} and R³ are hydrogen, L is —(CH₂)ₙ—, and n is 0, the preparation method for the compound as shown in Formula (II-2) is as follows:

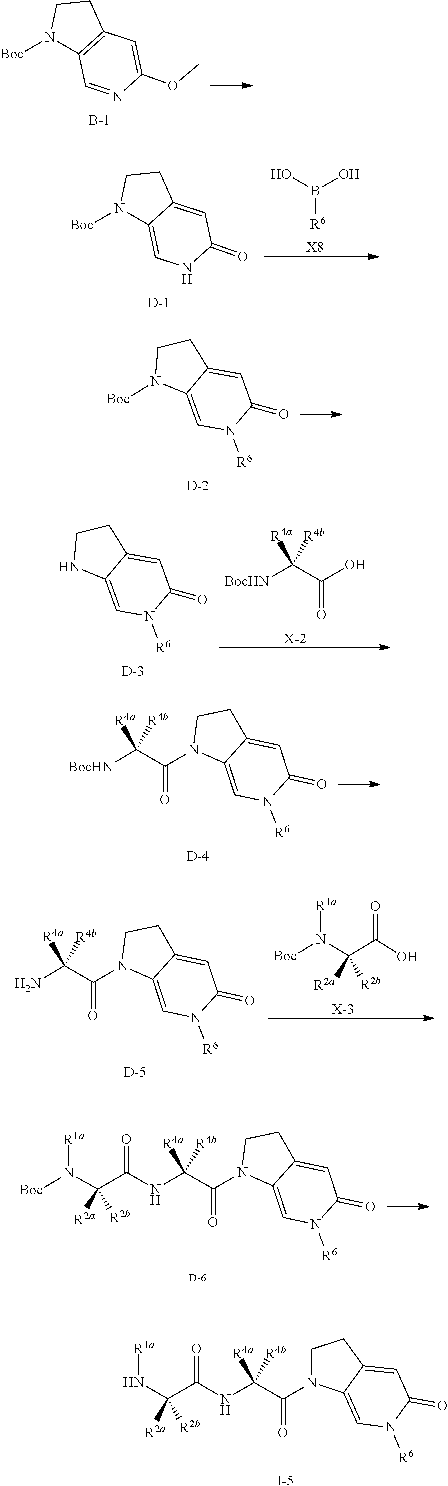

Compound D-1 is prepared from compound B-1 by dissolving compound B-1 in a solvent and adding NaSCH₅ for reaction. The reaction solvent is N,N-dimethylformamide (DMF), tetrahydrofuran (THF), 1,4-dioxane, dichloromethane (DCM), acetonitrile or diethyl ether, preferably N,N-dimethylformamide (DMF).

Compound D-2 is prepared from compound D-1 by dissolving compound D-1, X-8, a copper catalyst and an alkali in a solvent and reacting openly at room temperature. The reaction solvent is dichloromethane, tetrahydrofuran (THF), N,N-dimethylformamide (DMF), 1,4-dioxane, glycol dimethyl ether or acetonitrile, preferably N,N-dimethylformamide (DMF), preferably dichloromethane; the metal catalyst is copper sulfate, ketone iodide, cuprous iodide, copper chloride, cuprous chloride or copper acetate, preferably copper acetate; the alkali is triethylamine, pyridine or DIPEA, preferably a mixture of triethylamine and pyridine.

Compound D-3 is prepared from compound D-2 by dissolving D-2 in a solvent and adding them to a 1,4-dioxane solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably 1,4-dioxane.

Compound D-4 is prepared from compound D-3 by dissolving D-3 in a solvent, adding a condensing agent and then adding an alkali and compound X-2 for condensation reaction. The solvent is dichloromethane, tetrahydrofuran (THF), N,N-dimethylformamide (DMF), 1,4-dioxane, glycol dimethyl ether or acetonitrile, preferably N,N-dimethylformamide (DMF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU), 1-hydroxybenzotriazole (HOBT) orbenzotriazole-1-yl-oxytripyrrolidinophosphonium hexafluorophosphate (PyBop), preferably 2-(7-azabenzotriazol-1-yl)-N,N,N′,N′-tetramethyluronium hexafluorophosphate (HATU); the alkali is triethylamine (TEA), sodium bicarbonate, sodium carbonate, potassium carbonate or DIPEA, preferably triethylamine.

Compound D-5 is prepared from compound D-4 by dissolving D-4 in a solvent and adding them to a 1,4-dioxane solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably 1,4-dioxane.

Compound D-6 is prepared from compound D-5 by dissolving D-5 in a solvent, adding a condensing agent and then adding an alkali and compound X-3 for condensation reaction. The solvent is dichloromethane, tetrahydrofuran (THF), N,N-dimethylformamide (DMF), 1,4-dioxane, glycol dimethyl ether or acetonitrile, preferably N,N-dimethylformamide (DMF); the condensing agent is selected from N,N′-carbonyl diimidazole (CDI), 1-(3-dimethylaminopropyl)-3-ethyl-carbodiimid monohydrochloride (EDCI), N,N′-dicyclohexyl carbodiimide (DCC), N,N′-diisopropyl carbodiimide (DIC), 2-(7-azabenzotriazol-1-yl)-N,N,N′,N′-tetramethyluronium hexafluorophosphate (HATU), 4-(4,6-dimethoxy-1,3,5-triazin-2-yl)-4-methyl morpholinium chloride (DMTMM), o-benzotriazol-N,N,N′,N′-tetramethyluronium-hexafluophosphate (HBTU), 1-hydroxybenzotriazole (HOBT) orbenzotriazole-1-yl-oxytripyrrolidinophosphonium hexafluorophosphate (PyBop), preferably 1-(3-dimethylaminopropyl)-3-ethyl-carbodiimid monohydrochloride (EDCI) and 1-hydroxybenzotriazole (HOBT); the alkali is triethylamine, sodium bicarbonate, sodium carbonate, potassium carbonate or DIPEA, preferably DIPEA.

Target compound I-5 is prepared from compound D-6 by dissolving D-6 in a solvent and adding a 1,4-dioxane solution of hydrogen chloride for deprotection reaction. The reaction solvent is N,N-dimethylformamide (DMF), tetrahydrofuran (THF), 1,4-dioxane, dichloromethane (DCM), acetonitrile or ethyl acetate, preferably 1,4-dioxane.

The corresponding acid is salified with the target compound (I) prepared by the foregoing method to obtain a pharmaceutically acceptable salt of the pyridone compound.

In the second aspect of the present invention, the pharmaceutical composition comprises any of the foregoing pyridone compounds as well as a pharmaceutically acceptable carrier. The pharmaceutically acceptable carrier can be added to make common pharmaceutical preparations, such as tablets, capsules, syrups, suspensions or injections. Common pharmaceutical adjuvants such as flavors, sweeteners, liquid/solid fillers and thinners, can be added.

In the third aspect of the present invention, the pyridone compound and its pharmaceutical composition can be made into cIAP1, cIAP2 and XIAP inhibitor drugs to treat tumors, specifically ovarian cancer, breast cancer and other cancers.

Advantages: Compared with the prior art, the present invention has the following significant advantages:
(1) Such pyridone compounds can effectively inhibit the enzyme activity of cIAP1, cIAP2 and XIAP, with the optimal enzyme inhibition IC₅₀ value of less than 10 nM; and have an inhibitory effect on a variety of tumor cells, with the optimal tumor cell inhibition IC₅₀ value of less than 10 nM, reaching the nanomolar concentration level;
(2) Such pyridone compounds have excellent in vivo pharmacokinetic properties, significantly increase half-life, in vivo exposure level and bioavailability, and have significant advantages in druggability;
(3) Such pyridone compounds and their pharmaceutical compositions are widely used and can be made into anti-tumor drugs. The drugs can have a pharmaceutical effect at both the molecular level and the cellular level, and particularly, show more excellent in vivo pharmacokinetic properties.

### DETAILED DESCRIPTION

Below the technical solution of the present invention is further described in conjunction with embodiments.

The technical terms and abbreviations in this patent are explained as follows:
1,4-dioxane: 1,4-dioxane
DCM: dichloromethane
THF: tetrahydrofuran
DMF: N,N-dimethylformamide
DMSO: dimethyl sulfoxide
Boc₂O: di-tert-butyl dicarbonate
Oxirane: ethylene oxide
n-BuLi: n-butyl lithium
TMEDA: N,N,N′,N′-tetramethylenediamine
MsCl: methyl sulfonyl chloride
TEA: triethylamine
DIPEA: N,N-diisopropylethylamine
DMAP: 4-dimethylaminopyridine
TMEDA: tetramethylenediamine
LiHMDS: lithium bis(trimethylsilyl)amide
NaSCH₃: sodium methylmercaptan
CDI: N,N′-carbonyl diimidazole
EDCI: 1-(3-dimethylaminopropyl)-3-ethyl-carbodiimid monohydrochloride
DCC: N,N′-dicyclohexyl carbodiimide
DIC: N,N′-diisopropyl carbodiimide
HATU: 2-(7-azabenzotriazol-1-yl)-N,N,N′,N′-tetramethyluronium hexafluorophosphate
DMTMM: 4-(4,6-dimethoxy-1,3,5-triazin-2-yl)-4-methyl morpholinium chloride
HBTU: o-benzotriazol-N,N,N′,N′-tetramethyluronium-hexafluophosphate
PyBop: benzotriazole-1-yl-oxytripyrrolidinophosphonium hexafluorophosphate
DMTMM: 4-(4,6-dimethoxy-1,3,5-triazin-2-yl)-4-methyl morpholinium chloride
HOBT: 1-hydroxybenzotriazole
DTT: dithiothreitol
WST-8 kit: cytotoxicity/proliferation assay kit

In the embodiments described below, the conditions for reversed phase preparative chromatography are (brand and model of the preparative chromatograph: SHIMADZU LC 20AP; column: Agilent 10 Prep-C18, 250*21.2 mm, 10 μm; mobile phase A: aqueous solution containing 0.1% formic acid, mobile phase B: acetonitrile, detection wavelength: 214 nm, flow rate: 15 mL/min).

### Embodiment 1: Synthesis of (S)-N-((S)-2-(6-benzyl-7-oxo-2,3,6,7-tetrahydro-1H-pyrrolo[2,3-c]pyridine-1-yl)-1-cyclohexyl-2-oxoethyl)-2-(methylamino) propanamide (I-1-1)

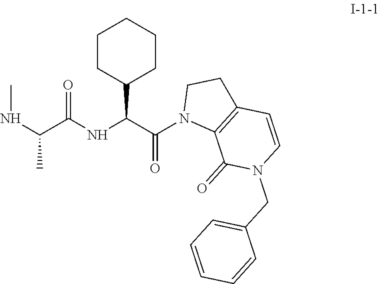 [SMILES: CN[C@@H](C)C(=O)N[C@H](C(=O)N1CCc2ccn(Cc3ccccc3)c(=O)c21)C1CCCCC1]

### Synthesis of (2-methoxypyridine-3-yl) tert-butyl carbamate (A-2)

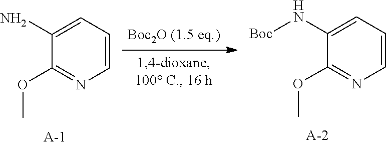

To a solution of A-1 (10.00 g, 80.6 mmol, 1.0 equivalent) in 1,4-dioxane (300 mL) was added di-tert-butyl dicarbonate (26.4 g, 120.9 mmol, 1.5 equivalents), and stirred at 100° C. for 10 h. The reaction mixture was concentrated in vacuo. The crude material was absorbed onto a plug of silica gel and purified by column chromatography to obtain product A-2 as the colorless oil (18 g, yield 99%).

LCMS: ESI-MS(+), [M+1]=225.1.

### Synthesis of (4-(2-hydroxyethyl)-2-methoxypyridine-3-yl) tert-butyl carbamate (A-3)

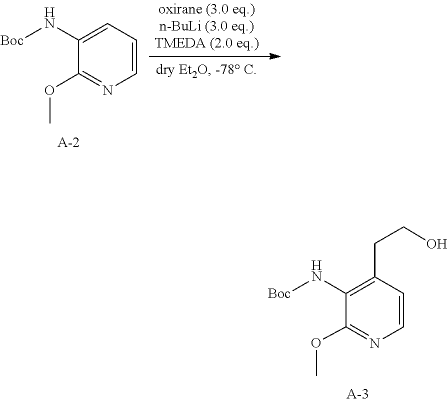

A-2 (26.00 g, 0.12 mol, 1.0 equivalent) and N,N,N′,N′-tetramethylenediamine (54.00 g, 0.46 mol, 2.0 equivalents) was dissolved in anhydrous ether (200 mL), and the reaction mixture was cool to −78° C. under nitrogen. n-Butyl lithium (225 mL, 1.6 mol/L, 0.36 mol, 3.0 equivalents) was dropwise added to the reaction medium slowly at −78° C. The mixture was stirred for 1 h at the same temperature, then ethylene oxide (116 mL, 0.35 mol, 3.0 equivalents) was added and stirred for another for 6 h at −78° C. the reaction solution was quenched with ice water (20 mL), and then added another ice water (180 mL) and stirred for 10 min. The reaction solution was extracted with ethyl acetate (200 mL*2). The combined organic extracts were washed with brine (180 mL), and dried over anhydrous sodium sulfate, filtered and concentrated under vacuum. The crude product was purified by silica gel column chromatography (petroleum ether:ethyl acetate=5:1) to obtain A-3 (8.80 g, yield 28%) and the recovered raw material A-2 (14.80 g).

¹H NMR (400 MHz, DMSO-d₆) δ 8.19 (s, 1H), 7.92 (d, J=5.2 Hz, 1H), 6.89 (d, J=5.2 Hz, 1H), 4.71 (t, J=5.1 Hz, 1H), 3.82 (s, 3H), 3.57 (td, J=7.1, 5.1 Hz, 2H), 2.68 (t, J=7.1 Hz, 2H), 1.41 (s, 9H).

LCMS: ESI-MS(+), [M+1]=269.1.

### Synthesis of 2-(3-((tert-butoxycarbonyl)amino)-2-methoxy-pyridine-4-yl)ethyl methanesulfonate (A-4)

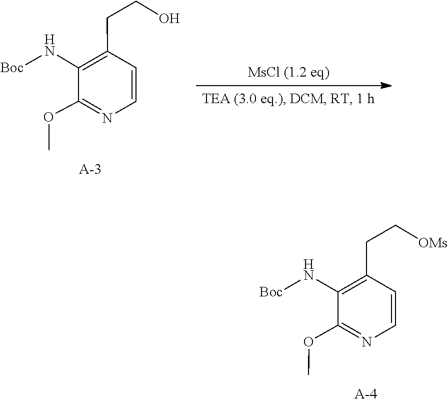

To a solution of A-3 (8.80 g, 32.8 mmol, 1.0 equivalent) in dichloromethane (50 mL) was added triethylamine (4.50 g, 39.4 mmol, 3.0 equivalents) and methyl sulfonyl chloride (4.50 g, 39.4 mmol, 1.2 equivalents) at 0° C., respectively. The reaction solution was stirred at room temperature for 2 h. The reaction solution was quenched with ice water (50 mL), and extracted with dichloromethane (20 mL*2). The combined organic layers were dried with anhydrous sodium sulfate, filtered, and concentrated under vacuum. The crude products was purified by silica gel column chromatography (petroleum ether:ethyl acetate=10:1) to obtain A-4 (10.50 g, yield 92%).

LCMS: ESI-MS(+), [M+1]=347.1.

### Synthesis of 7-methoxy-2,3-dihydro-1H-pyrrolo[2,3-c] pyridine-1-tert-butyl formate (A-5)

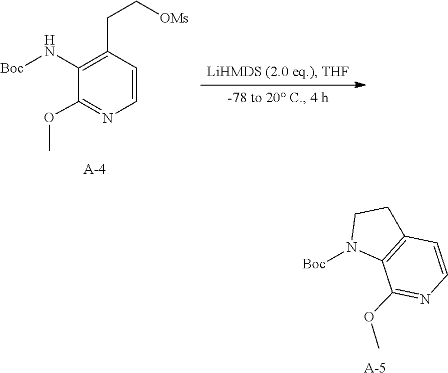

To a solution of A-4 (10.50 g, 30.34 mmol, 1.0 equivalent) in tetrahydrofuran (50 mL) was added lithium bis(trimethylsilyl)amide (60.7 mL, 60.7 mmol, 2.0 equivalents) dropwise at −78° C. under nitrogen protection. Then, the reaction solution was stirred at room temperature for 4 h. The reaction solution was quenched with ice water (200 mL) and extracted with ethyl acetate (200 mL*3). The organic layer was washed with brine (100 mL*2), dried over anhydrous sodium sulfate, filtered, and concentrated in vacuo to obtain a crude product, which was purified by silica gel column chromatography (petroleum ether:ethyl acetate=2:1) to obtain A-5 (5.90 g, yield 77.6%).

¹H NMR (400 MHz, DMSO-d₆) δ 7.85 (d, J=4.9 Hz, 1H), 6.94 (d, J=4.9 Hz, 1H), 3.94 (t, J=8.0 Hz, 2H), 3.87 (s, 3H), 2.99 (t, J=8.0 Hz, 2H), 1.43 (s, 9H).

LCMS: ESI-MS(+), [M+1]=251.1.

### Synthesis of 7-oxo-2,3,6,7-tetrahydro-1H-pyrrolo[2,3-c]pyridine-1-tert-butyl formate (A-6)

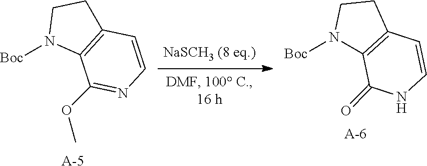

A suspension of A-5 (5.90 g, 23.6 mmol, 1.0 equivalent) and sodium methylmercaptan (13.2 g, 189 mmol, 8.0 equivalents) in N,N-dimethylformamide (30 mL) was heated to 100° C. under nitrogen, and stirred for 16 h. N,N-dimethylformamide was removed under reduced pressure. To the residue was added ethyl acetate (100 mL), and filtered to remove the insoluble impurities. The filtrate was concentrated, and further purified by silica gel column chromatography (petroleum ether:ethyl acetate=2:1) to obtain A-6 (3.95 g, yield 71%).

¹H NMR (400 MHz, DMSO-d₆) δ 11.44 (s, 1H), 7.15 (d, J=6.3 Hz, 1H), 6.21 (d, J=6.3 Hz, 1H), 3.86 (t, J=8.2 Hz, 2H), 2.89 (t, J=8.2 Hz, 2H), 1.42 (s, 9H).

LCMS: ESI-MS (+), [M+1]=237.1.

### Synthesis of 6-benzyl-7-oxo-2,3,6,7-tetrahydro-1H-pyrrolo[2,3-c]pyridine-1-tert-butyl formate (A-7-1)

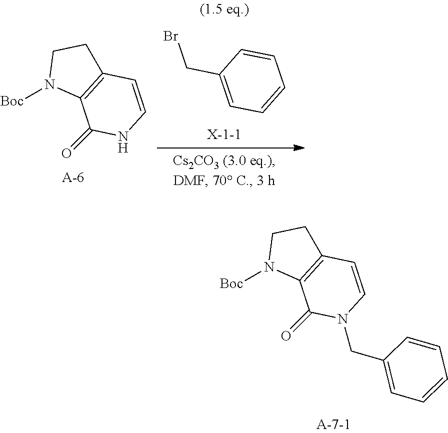

A suspension of A-6 (1.50 g, 6.4 mmol, 1.0 equivalent), cesium carbonate (1.63 g, 9.5 mmol, 1.5 equivalents) and X-1-1 (6.21 g, 19.1 mmol, 1.5 equivalents) in N,N-dimethylformamide (25 mL) was stirred at 70° C. for 4 h. The mixture was cooled to room temperature and added ethyl acetate (100 mL*3) and water (150 mL). The organic phase was washed with brine (100 mL*2), dried over anhydrous sodium sulfate, concentrated and the crude product was purified by silica gel column chromatography (petroleum ether:ethyl acetate=1:1) to obtain A-7-1 (1.75 g, yield 85%).

¹H NMR (400 MHz, DMSO-d₆) δ 7.58 (d, J=6.7 Hz, 1H), 7.34-7.23 (m, 5H), 6.25 (d, J=6.7 Hz, 1H), 5.12 (s, 2H), 3.88 (t, J=8.2 Hz, 2H), 2.90 (t, J=8.1 Hz, 2H), 1.37 (s, 9H).

LCMS: ESI-MS (+), [M+1]=326.7, [2M+23]=675.3.

### Synthesis of 6-benzyl-1,2,3,6-tetrahydro-7H-pyrrolo[2,3-c]pyridine-7-one (A-8-1)

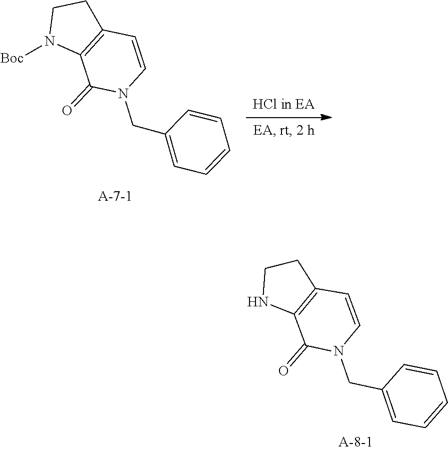 [SMILES: CC(C)(C)OC(=O)N1CCc2ccn(Cc3ccccc3)c(=O)c21 ; O=c1c2c(ccn1Cc1ccccc1)CCN2]

To a solution of A-7-1 (1.75 g, 5.4 mmol, 1.0 equivalent) in ethyl acetate (5 mL) was added hydrogen chloride in ethyl acetate solution (4 mol/L, 20 mL) dropwise, and stirred at room temperature for 2 h and concentrated under vacuum. The residue was diluted with saturated sodium bicarbonate solution (30 mL) and extracted with ethyl acetate (50 mL*3). The organic layer was washed with brine (50 mL), dried over anhydrous sodium sulfate, and concentrated to obtain a crude product A-8-1 (1.20 g), which was directly used for subsequent reaction.

LCMS: ESI-MS (+), [M+1]=227.2.

### Synthesis of (S)-(2-(6-benzyl-7-oxo-2,3,6,7-tetrahydro-1H-pyrrolo[2,3-c]pyridine-1-yl)-1-cyclohexyl-2-oxyethyl)tert-butyl carbamate (A-9-1)

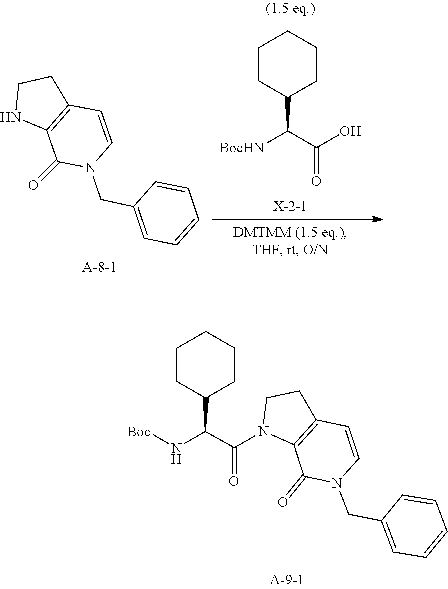 [SMILES: O=c1c2c(ccn1Cc1ccccc1)CCN2 ; CC(C)(C)OC(=O)N[C@H](C(=O)O)C1CCCCC1 ; CC(C)(C)OC(=O)N[C@H](C(=O)N1CCc2ccn(Cc3ccccc3)c(=O)c21)C1CCCCC1]

To a solution of A-8-1 (1.20 g, 5.3 mmol, 1.0 equivalent) and X-2-1 (2.05 g, 8.0 mmol, 1.5 equivalents) in tetrahydrofuran (20 mL) was added 4-(4,6-dimethoxy-1,3,5-triazin-2-yl)-4-methyl morpholinium chloride (2.20 g, 8 mmol, 1.5 equivalents) and stirred at room temperature for 16 h. The reaction was diluted with water (20 mL) and extracted with ethyl acetate (30 mL*3). The organic phase was washed with brine (50 mL), dried over anhydrous sodium sulfate and concentrated. The crude product was purified by silica gel column chromatography (petroleum ether:ethyl acetate=1:1) to obtain A-9-1 (1.50 g, yield 61%).

¹H NMR (400 MHz, DMSO-d₆) δ 7.66 (d, J=6.6 Hz, 1H), 7.33-7.27 (m, 5H), 6.37 (d, J=6.4 Hz, 1H), 5.30 (d, J=14.8 Hz, 1H), 5.09 (d, J=14.3 Hz, 1H), 4.26 (s, 1H), 3.97-3.90 (m, 1H), 2.98-2.89 (m, 1H), 2.86-2.77 (m, 1H), 1.67-1.43 (m, 6H), 1.34 (s, 9H), 1.07-0.92 (m, 5H).

LCMS: ESI-MS (+), [M+1]=466.2.

### Synthesis of (S)-1-(2-amino-2-cyclohexyl acetyl)-6-benzyl-1,2,3,6-tetrahydro-7H-pyrrolo[2,3-c]pyridine-7-one (A-10-1)

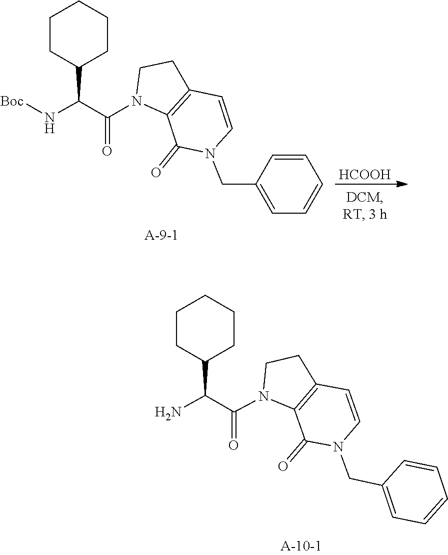 [SMILES: CC(C)(C)OC(=O)N[C@H](C(=O)N1CCc2ccn(Cc3ccccc3)c(=O)c21)C1CCCCC1 ; "HCOOH" ; N[C@H](C(=O)N1CCc2ccn(Cc3ccccc3)c(=O)c21)C1CCCCC1]

To a solution of A-9-1 (1.51 g, 3.25 mmol, 1.0 equivalent) in dichloromethane (3 mL) was added formic acid (15 mL) and stirred at room temperature for 3 h. The reaction solution was diluted with water (20 mL), adjusted to pH 8 with saturated sodium bicarbonate solution (250 mL), then extracted with ethyl acetate (50 mL*6). The organic phase was washed with brine (50 mL), dried over anhydrous sodium sulfate and then concentrated. The crude product was purified by silica gel column chromatography (dichloromethane:methanol=20:1) to obtain A-10-1 (1.09 g, yield 92.4%).

LCMS: ESI-MS (+), [M+1]=366.2.

### Synthesis of ((S)-1-(((S)-2-(6-benzyl-7-oxo-2,3,6,7-tetrahydro-1H-pyrrolo[2,3-c]pyridine-1-yl)-1-cyclohexyl-2-oxyethyl)amino)-1-oxopropyl-2-yl)(methyl)tert-butyl carbamate (A-11-1)

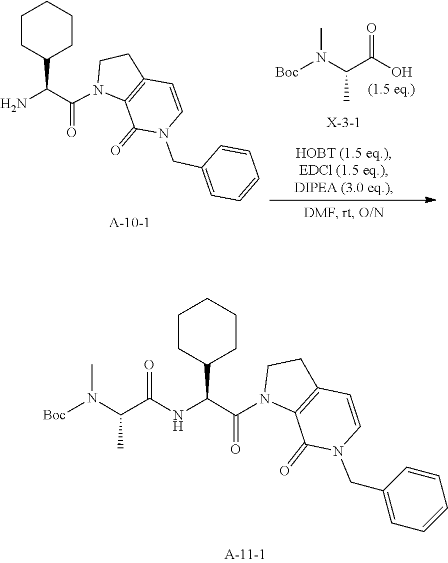 [SMILES: N[C@H](C(=O)N1CCc2ccn(Cc3ccccc3)c(=O)c21)C1CCCCC1 ; C[C@@H](C(=O)O)N(C)C(=O)OC(C)(C)C ; C[C@@H](C(=O)N[C@H](C(=O)N1CCc2ccn(Cc3ccccc3)c(=O)c21)C1CCCCC1)N(C)C(=O)OC(C)(C)C]

A suspension of A-10-1 (1.09 g, 3.0 mmol, 1.0 equivalent), X-3-1 (909 mg, 4.5 mmol, 1.5 equivalents), 1-hydroxybenzotriazole (605 mg, 4.5 mmol, 1.5 equivalents), 1-(3-dimethylaminopropyl)-3-ethyl-carbodiimid monohydrochloride (860 mg, 4.5 mmol, 1.5 equivalents) and N,N-diisopropylethylamine (1.156 g, 9.0 mmol, 3.0 equivalents) in N,N-dimethylformamide (15 mL) was stirred at room temperature for 16 h. The reaction was quenched with water (100 mL) and extracted with ethyl acetate (30 mL*3). The organic phase was washed with brine (50 mL), dried with anhydrous sodium sulfate and then concentrate. The crude product was purified by silica gel column chromatography (petroleum ether:ethyl acetate=1:1) to obtain A-11-1 (1.23 g, yield 75%).

LCMS: ESI-MS (+), [M+1]=551.2.

### Synthesis of (S)-N-((S)-2-(6-benzyl-7-oxo-2,3,6,7-4H-1H-pyrrolo [2,3-c] pyridine-1-yl)-1-cyclohexyl-2-oxoethyl)-2-(methylamino) propanamide (I-1-1)

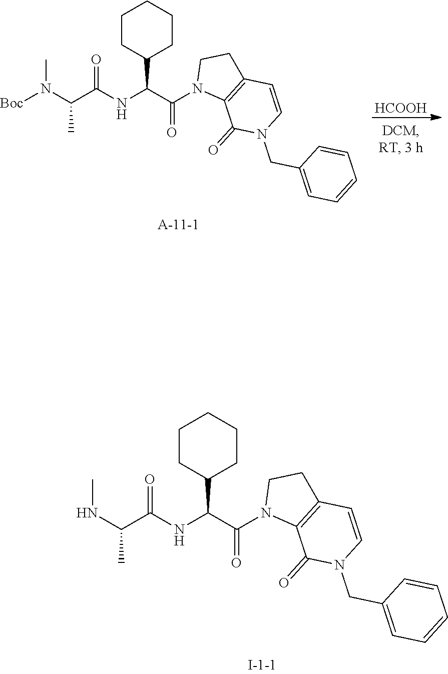 [SMILES: C[C@@H](C(=O)N[C@H](C(=O)N1CCc2ccn(Cc3ccccc3)c(=O)c21)C1CCCCC1)N(C)C(=O)OC(C)(C)C ; "HCOOH" ; CN[C@@H](C)C(=O)N[C@H](C(=O)N1CCc2ccn(Cc3ccccc3)c(=O)c21)C1CCCCC1]

To a solution of A-11-1 (600 mg, 1.091 mmol, 1.0 equivalent) in dichloromethane (1 mL) was added formic acid (5 mL), and stirred at room temperature for 16 h. The reaction solution was concentrated and then purified by reversed phase preparative liquid chromatography to obtain I-1-1 (403 mg, yield 82.1%).

¹H NMR (400 MHz, DMSO-d₆) δ 8.24 (s, 1H), 7.94 (d, J=8.9 Hz, 1H), 7.68 (d, J=6.7 Hz, 1H), 7.38-7.22 (m, 5H), 6.38 (d, J=6.7 Hz, 1H), 5.33 (d, J=14.4 Hz, 1H), 5.07 (d, J=14.5 Hz, 1H), 4.25 (s, 1H), 3.97-3.94 (d, 1H), 3.12 (q, J=6.8 Hz, 1H), 3.01-2.89 (m, 1H), 2.88-2.76 (m, 1H), 2.24 (s, 3H), 1.67-1.45 (m, 6H), 1.12 (d, J=6.8 Hz, 3H), 1.08-0.84 (m, 5H).

LCMS: ES-MS(+), [M+1]=5451.2.

The following compounds are prepared by referring to the preparation method in Embodiment 1:

| Embodiment | No. | ¹H-NMR | MS |
|---|---|---|---|
| Embodiment | I-1-2 | ¹H NMR (400 MHz, DMSO-d₆) δ 8.16 (s, | ESI-MS: |
| 2 | | 1H), 8.10 (d, J = 8.4 Hz, 1H), 7.72 (d, J = | [M + H]⁺ = |
| | | 6.7 Hz, 1H), 7.42-7.36 (m, 2H), 7.15 (t, | 469.15 |
| | | J = 8.8 Hz, 2H), 6.39 (d, J = 6.7 Hz, 1H), | |
| | | 5.43 (s, 1H), 5.28 (d, J = 14.1 Hz, 1H), | |
| | | 5.06 (d, J = 14.4 Hz, 1H), 4.23 (s, 1H), | |
| | | 4.00-3.92 (m, 1H), 3.37 (d, J = 6.7 Hz, | |
| | | 1H), 2.96 (dd, J = 16.7, 8.4 Hz, 1H), | |
| | | 2.861-2.785 (m, J = 11.9, 10.4, 4.6 Hz, | |
| | | 1H), 2.33 (s, 3H), 1.66-1.44 (m, 6H), 1.18 | |
| | | (d, J = 6.8 Hz, 3H), 1.047-0.837 (m, 5H). | |
| Embodiment | I-1-3 | ¹H NMR (400 MHz, DMSO-d₆) δ 8.21 (d, | ESI-MS: |
| 3 | | J = 8.6 Hz, 1H), 8.18 (s, 1H), 7.73 (d, J = | [M + H]⁺ = |
| | | 6.7 Hz, 1H), 7.39 (dd, J = 8.5, 5.6 Hz, | 469.15 |
| | | 2H), 7.15 (t, J = 8.9 Hz, 2H), 6.39 (d, J = | |
| | | 6.7 Hz, 1H), 5.40 (s, 1H), 5.28 (d, J = 14.3 | |
| | | Hz, 1H), 5.06 (d, J = 14.3 Hz, 1H), 4.24 | |
| | | (s, 1H), 3.96 (q, J = 10.8 Hz, 1H), 3.44 (q, | |
| | | J = 6.8 Hz, 1H), 2.98-2.90 (m, 1H), 2.87- | |
| | | 2.80 (m, 1H), 2.32 (s, 3H), 1.69-1.44 (m, | |
| | | 6H), 1.21 (d, J = 6.8 Hz, 3H), 1.09-0.84 | |
| | | (m, 5H). | |
| Embodiment | I-1-4 | ¹H NMR (400 MHz, DMSO-d₆) δ 7.97 (d, | ESI-MS: |
| 4 | | J = 8.8 Hz, 1H), 7.41-7.18 (m, 6H), 6.24 | [M + H]⁺ = |
| | | (d, J = 6.8 Hz, 1H), 5.44 (br, 1H), 4.31- | 465.20 |
| | | 4.18 (m, 2H), 4.16-4.06 (m, 1H), 3.96 (q, | |
| | | J = 9.2 Hz, 1H), 3.13 (q, J = 6.8 Hz, 1H), | |
| | | 3.02-2.87 (m, 3H), 2.85-2.75 (m, 1H), | |
| | | 2.25 (s, 3H), 1.75-1.46 (m, 6H), 1.13 (d, | |
| | | J = 6.8 Hz, 3H), 1.11-0.88 (m, 5H). | |
| Embodiment | I-1-5 | ¹H NMR (400 MHz, DMSO-d₆) δ | ESI-MS: |
| 5 | | 8.178.11 (m, 1H), 7.67 (d, J = 6.8 Hz, | [M + H]⁺ = |
| | | 1H), 7.32-7.22 (m, 2H), 7.04 (td, J = 8.4, | 487.35 |
| | | 2.4 Hz, 1H), 6.41 (d, J = 6.8 Hz, 1H), 5.29 | |
| | | (d, J = 14.8 Hz, 1H), 5.09 (d, J = 14.8 Hz, | |
| | | 1H), 4.23 (br, 1H), 4.02-3.89 (m, 2H), | |
| | | 3.43 (q, J = 6.8 Hz, 1H), 3.012.78 (m, | |
| | | 2H), 2.33 (s, 3H), 1.681.36 (m, 6H), 1.20 | |
| | | (d, J = 6.8 Hz, 3H), 1.09-0.86 (m, 5H). | |
| Embodiment | I-1-6 | ¹H NMR (400 MHz, DMSO-d₆) δ 7.92 (d, | ESI-MS: |
| 6 | | J = 8.8 Hz, 1H), 7.75 (d, J = 6.8 Hz, 1H), | [M + H]⁺ = |
| | | 7.69 (d, J = 8.0 Hz, 2H), 7.50 (d, J = 8.0 | 519.15 |
| | | Hz, 2H), 6.42 (d, J = 6.8 Hz, 1H), 5.37 (d, | |
| | | J = 14.8 Hz, 1H), 5.20 (d, J = 14.8 Hz, | |
| | | 1H), 4.23 (br, 1H), 4.02-3.90 (m, 2H), | |
| | | 3.10 (q, J = 6.8 Hz, 1H), 3.012.90 (m, | |
| | | 1H), 2.88-2.77 (m, 1H), 2.23 (s, 3H), | |
| | | 1.69-1.39 (m, 6H), 1.11 (d, J = 6.8 Hz, | |
| | | 3H), 1.05-0.83 (m, 5H). | |
| Embodiment | I-1-7 | ¹H NMR (400 MHz, DMSO-d₆) δ 8.09 (d, | ESI-MS: |
| 7 | | J = 8.4 Hz, 1H), 7.81 (d, J = 8.4 Hz, 2H), | [M + H]⁺ = |
| | | 7.74 (d, J = 6.8 Hz, 1H), 7.46 (d, J = 8.4 | 476.20 |
| | | Hz, 2H), 6.43 (d, J = 6.8 Hz, 1H), 5.35 (d, | |
| | | J = 15.2 Hz, 1H), 5.20 (d, J = 15.2 Hz, | |
| | | 1H), 4.21 (br, 1H), 3.99 (q, J = 10.4 Hz, | |
| | | 1H), 3.33 (q, J = 6.8 Hz, 2H), 3.01-2.90 | |
| | | (m, 1H), 2.89-2.79 (m, 1H), 2.30 (s, 3H), | |
| | | 1.69-1.41 (m, 6H), 1.17 (d, J = 6.8 Hz, | |
| | | 3H), 1.07-0.84 (m, 5H) | |
| Embodiment | I-1-8 | ¹H NMR (400 MHz, DMSO-d₆) δ 8.51 (d, | ESI-MS: |
| 8 | | J = 4.4 Hz, 1H), 8.02 (d, J = 8.0 Hz, 1H), | [M + H]⁺ = |
| | | 7.71 (d, J = 6.4 Hz, 1H), 7.21 (d, J = 4.8 | 452.30 |
| | | Hz, 2H), 6.44 (d, J = 6.4 Hz, 1H), 5.31 (d, | |
| | | J = 15.4 Hz, 1H), 5.15 (d, J = 15.4 Hz, | |
| | | 1H), 4.04-3.94 (m, 2H), 3.25-3.15 (m, | |
| | | 1H), 3.03-2.79 (m, 2H), 2.25 (s, 3H), | |
| | | 1.71-1.42 (m, 6H), 1.13 (d, J = 6.8 Hz, | |
| | | 3H), 1.08-0.84 (m, 5H) | |
| Embodiment | I-1-9 | ¹H NMR (400 MHz, Methanol-d₄) δ 8.39 | ESI-MS: |
| 9 | | (s, 1H), 8.03 (t, J = 7.2 Hz, 1H), 7.73 (d, | [M + H]⁺ = |
| | | J = 6.8 Hz, 1H), 7.12-6.98 (m, 1H), 6.48 (d, | 470.15 |
| | | J = 6.8 Hz, 1H), 5.36-5.19 (m, 2H), 4.43 | |
| | | (br, 1H), 4.08 (q, J = 9.2 Hz, 1H), 3.86 (d, | |
| | | J = 6.4 Hz, 1H), 3.31 (s, 1H), 3.14-2.88 | |
| | | (m, 2H), 2.65 (s, 3H), 1.78-1.55 (m, 6H), | |
| | | 1.47 (d, J = 6.4 Hz, 3H), 1.21-0.99 (m, 5H). | |
| Embodiment | I-1-10 | ¹H NMR (400 MHz, Methanol-d₄) δ 7.81 | ESI-MS: |
| 10 | | (t, J = 7.6 Hz, 1H), 7.72 (d, J = 6.4 Hz, | [M + H]⁺ = |
| | | 1H), 7.44-7.31 (m, 2H), 6.52 (d, J = 6.8 | 452.30 |
| | | Hz, 1H), 5.57-5.22 (m, 2H), 4.50 (br, 1H), | |
| | | 4.10 (q, J = 9.6 Hz, 1H), 3.89-3.74 (m, | |
| | | 1H), 3.24-3.07 (m, 1H), 3.04-2.90 (m, | |
| | | 1H), 2.63 (s, 3H), 1.841.56 (m, 6H), 1.46 | |
| | | (d, J = 6.8 Hz, 3H), 1.26-1.00 (m, 5H). | |
| Embodiment | I-1-11 | ¹H NMR (400 MHz, Methanol-d₄) δ 9.06 | ESI-MS: |
| 11 | | (s, 1H), 8.71 (d, J = 5.2 Hz, 1H), 8.54 (br, | [M + H]⁺ = |
| | | 1H), 7.68 (d, J = 6.8 Hz, 1H), 7.43 (d, J = | 453.30 |
| | | 4.8 Hz, 1H), 6.52 (d, J = 6.8 Hz, 1H), 5.33 | |
| | | (t, J = 13.2 Hz, 2H), 4.45 (br, 1H), 4.10 (q, | |
| | | J = 9.2 Hz, 1H), 3.56 (q, J = 6.8 Hz, 1H), | |
| | | 3.30 (s, 1H), 3.20-3.06 (m, 1H), 3.03-2.91 | |
| | | (m, 1H), 2.50 (s, 3H), 1.72-1.61 (m, 6H), | |
| | | 1.35 (d, J = 6.8 Hz, 3H), 1.22-0.98 (m, 5H). | |
| Embodiment | I-1-12 | ¹H NMR (400 MHz, Methanol-d₄) δ 8.90 | ESI-MS: |
| 12 | | (s, 1H), 8.74 (s, 1H), 8.52-8.42 (m, 1H), | [M + H]⁺ = |
| | | 7.97-7.88 (m, 1H), 7.77 (d, J = 6.7 Hz, | 452.20 |
| | | 1H), 6.52 (d, J = 6.7 Hz, 1H), 5.42 (s, | |
| | | 2H), 4.42 (s, 1H), 4.14 (q, 1H), 3.88 (q, | |
| | | J = 7.1 Hz, 1H), 3.08 (s, 1H), 2.97 (d, J = | |
| | | 4.6 Hz, 1H), 2.66 (s, 3H), 1.78-1.58 (m, | |
| | | 7H), 1.45 (d, J = 6.9 Hz, 3H), 1.25-1.03 | |
| | | (m, 5H). | |
| Embodiment | I-1-13 | ¹H NMR (400 MHz, MeOD) δ 8.51 (s, | ESI-MS: |
| 13 | | 1H), 7.71 (d, J = 6.7 Hz, 1H), 7.34 (dd, | [M + H]⁺ = |
| | | J = 8.4, 6.7 Hz, 1H), 6.60-6.38 (m, 3H), | 485.1 |
| | | 5.24 (d, J = 14.2 Hz, 1H), 5.11 (d, J = | |
| | | 14.1 Hz, 1H), 4.46 (s, 1H), 4.04 (q, J = 9.9 | |
| | | Hz, 1H), 3.76 (q, J = 6.9 Hz, 1H), 3.08 | |
| | | (dt, J = 18.2, 9.5 Hz, 1H), 2.90 (ddd, J = | |
| | | 17.0, 9.2, 3.8 Hz, 1H), 2.60 (s, 3H), 1.80- | |
| | | 1.52 (m, 6H), 1.44 (d, J = 6.9 Hz, 3H), | |
| | | 1.09 (d, J = 11.7 Hz, 5H). | |
| Embodiment | I-1-14 | ¹H NMR (400 MHz, MeOD) δ 7.52 (s, | ESI-MS: |
| 14 | | 1H), 7.38 (q, J = 10.5, 8.6 Hz, 6H), 7.26 | [M + H]⁺ = |
| | | (d, J = 6.8 Hz, 1H), 7.18 (d, J = 7.2 Hz, | 527. |
| | | 4H), 6.42 (d, J = 6.9 Hz, 1H), 5.35 (d, J = | |
| | | 4.5 Hz, 1H), 4.54 (d, J = 16.6 Hz, 1H), | |
| | | 4.07 (q, J = 9.5 Hz, 1H), 3.60 (d, J = 6.4 | |
| | | Hz, 1H), 3.12 (d, J = 9.5 Hz, 1H), 2.99- | |
| | | 2.86 (m, 1H), 2.53 (s, 3H), 2.19 (t, J = 7.5 | |
| | | Hz, 1H), 2.03 (d, J = 5.3 Hz, 1H), 1.66 (s, | |
| | | 6H), 1.39 (d, J = 6.7 Hz, 3H), 1.19-1.04 | |
| | | (m, 5H). | |

### Embodiment 15: Synthesis of (S)-N-((S)-1-cyclohexyl-2-(6-(4-fluorobenzyl)-5-oxo-2,3,5,6-tetrahydro-1H-pyrrolo[2,3-c]pyridine-1-yl)-2-oxyethyl)-2-(methylamino)propanamide (I-2-1)

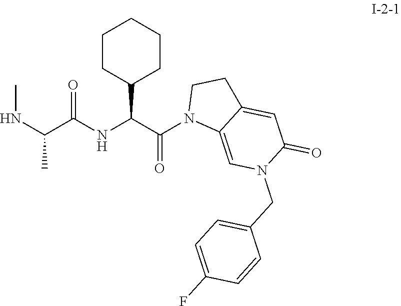 [SMILES: CN[C@@H](C)C(=O)N[C@H](C(=O)N1CCc2cc(=O)n(Cc3ccc(F)cc3)cc21)C1CCCCC1]

### Synthesis of 6-(4-fluorobenzyl)-5-oxo-2,3,5,6-tetrahydro-1H-pyrrolo[2,3-c]pyridine-1-tert-butyl formate (B-2-1)

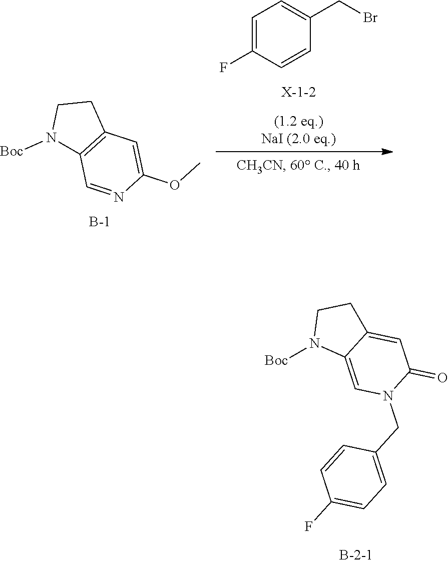

A suspension of 5-methoxy-2,3-dihydro-1H-pyrrolo[2,3-c]pyridine-1-tert-butyl carboxylate (B-1) (200 mg, 0.80 mmol, 1.0 equivalent) and sodium iodide (240 mg, 1.60 mmol, 2.0 equivalents) in acetonitrile (10 mL) was added 1-(bromomethyl)-4-fluorobenzene (X-1-2) (180 mg, 0.96 mmol, 1.2 equivalents) and stirred at 60° C. for 40 h. The reaction mixture was diluted with water (50 mL) and extracted with ethyl acetate (20 mL*3). The organic layer was washed with water (30 mL*2) and brine (50 mL) successively, dried over anhydrous sodium sulfate and then concentrated under reduced pressure. The crude product was further purified by silica gel column chromatography (petroleum ether/ethyl acetate=10/1, v/v) to obtain B-2-1 (222 mg, yield 80%).

¹H NMR (400 MHz, DMSO-d₆) δ 7.79 (s, 1H), 7.36-7.29 (m, 2H), 7.15 (t, J=8.7 Hz, 2H), 6.36 (s, 1H), 5.07 (s, 2H), 3.79 (t, J=8.2 Hz, 2H), 2.99 (t, J=8.2 Hz, 2H), 1.44 (s, 9H).

LCMS: ESI-MS(+), [M+1]=345.2.

### Synthesis of 6-(4-fluorobenzyl)-1,2,3,6-tetrahydro-5H-pyrrolo[2,3-c]pyridine-5-one (B-3-1)

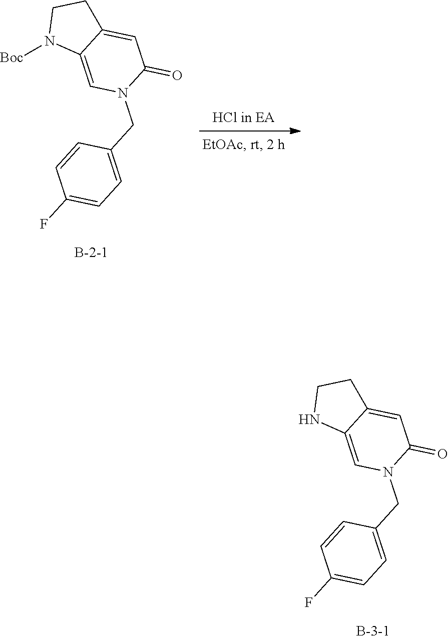 [SMILES: CC(C)(C)OC(=O)N1CCc2cc(=O)n(Cc3ccc(F)cc3)cc21 ; O=c1cc2c(cn1Cc1ccc(F)cc1)NCC2]

To a solution of 6-(4-fluorobenzyl)-5-oxo-2,3,5,6-tetrahydro-1H-pyrrolo[2,3-c]pyridine-1-tert-butyl carboxylate (B-2-1) (222 mg, 0.64 mmol, 1.0 equivalent) in ethyl acetate (1 mL) was added hydrogen chloride in ethyl acetate solution (4 mL), and stirred at room temperature for 2 h and then concentrated in vacuo. The residue was diluted with water (5 mL), adjusted pH 10 with sodium hydroxide solution (2 mol/L) and then extracted with ethyl acetate (10 mL*3). The combined organic layer was washed with water (10 mL) and brine (10 mL) successively, dried over anhydrous sodium sulfate, and then concentrated to obtain a crude product B-3-1 (147 mg, yield 94%), which was directly used at the next step without any further purification.

LCMS: ESI-MS(+), [M+1]=245.1.

### Synthesis of (S)-(1-cyclohexyl-2-(6-(4-fluorobenzyl)-5-oxo-2,3,5,6-tetrahydro-1H-pyrrolo[2,3-c]pyridine-1-yl)-2-oxyethyl)tert-butyl carbamate (B-4-1)

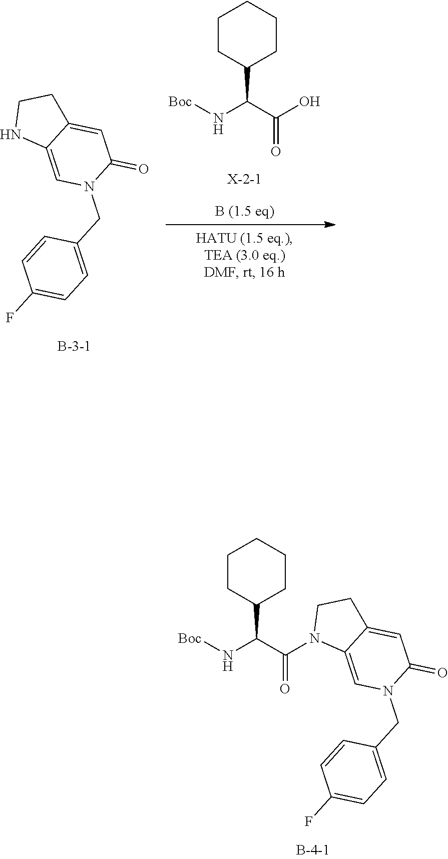 [SMILES: O=c1cc2c(cn1Cc1ccc(F)cc1)NCC2 ; CC(C)(C)OC(=O)N[C@H](C(=O)O)C1CCCCC1 ; CC(C)(C)OC(=O)N[C@H](C(=O)N1CCc2cc(=O)n(Cc3ccc(F)cc3)cc21)C1CCCCC1]

To a suspension of 6-(4-fluorobenzyl)-1,2,3,6-tetrahydro-5H-pyrrolo[2,3-c]pyridine-5-one (B-3-1) (147 mg, 0.60 mmol, 1.0 equivalent), (S)-2-((tert-butoxycarbonyl)amino)-2-cyclohexylacetic acid (X-2-1) (231 mg, 0.90 mmol, 1.5 equivalents) and triethylamine (182 mg, 1.80 mmol, 3.0 equivalents) in N,N-dimethylformamide (5 mL) was added 2-(7-azabenzotriazol-1-yl)-N,N,N′,N′-tetramethyluronium hexafluorophosphate (HATU, 342 mg, 0.90 mmol, 1.5 equivalents), and stirred at room temperature for 16 h. The reaction mixture was then diluted with water (30 mL) and extracted with ethyl acetate (20 mL*3). The combined organic layer was washed with water (30 mL*2) and brine (30 mL) successively, dried over anhydrous sodium sulfate and concentrated. The crude product was purified by silica gel column chromatography (petroleum ether/ethyl acetate=8/1, v/v) to obtain B-4-1 (280 mg, yield 95%).

¹H NMR (400 MHz, DMSO-d₆) δ 8.29 (s, 1H), 7.32 (dd, J=8.3, 5.5 Hz, 2H), 7.13 (dt, J=16.6, 8.4 Hz, 3H), 6.38 (s, 1H), 5.13 (d, J=14.4 Hz, 1H), 5.01 (d, J=14.4 Hz, 1H), 4.20 (q, J=9.0, 8.6 Hz, 1H), 4.08 (dt, J=25.4, 8.6 Hz, 2H), 3.10 (t, J=8.3 Hz, 2H), 1.76-1.47 (m, 6H), 1.35 (s, 9H), 1.12 (t, J=10.6 Hz, 5H).

LCMS: ESI-MS(+), [M+1]=484.3.

### Synthesis of (S)-1-(2-amino-2-cyclohexyl acetyl)-6-(4-fluorobenzyl)-1,2,3,6-tetrahydro-5H-pyrrolo[2,3-c]pyridine-5-one (B-5-1)

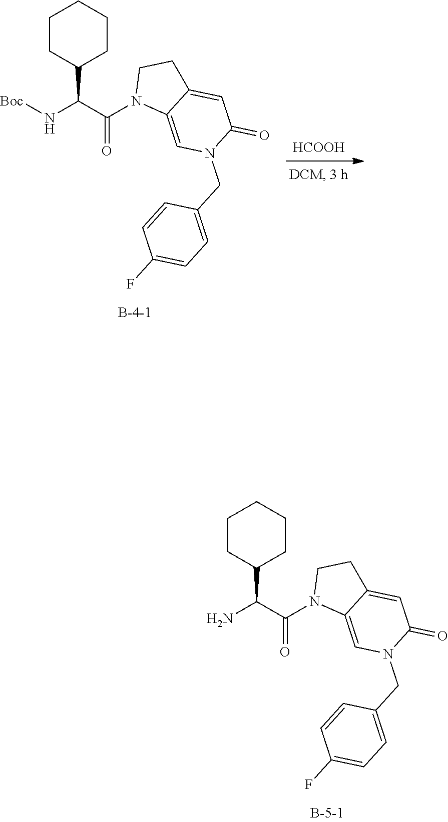 [SMILES: CC(C)(C)OC(=O)N[C@H](C(=O)N1CCc2cc(=O)n(Cc3ccc(F)cc3)cc21)C1CCCCC1 ; "HCOOH" ; N[C@H](C(=O)N1CCc2cc(=O)n(Cc3ccc(F)cc3)cc21)C1CCCCC1]

To a solution of (S)-(1-cyclohexyl-2-(6-(4-fluorobenzyl)-5-oxo-2,3,5,6-tetrahydro-1H-pyrrolo[2,3-c]pyridine-1-yl)-2-oxyethyl) tert-butyl carbamate (B-4-1) (250 mg, 0.51 mmol, 1.0 equivalent) in dichloromethane (1 mL) was added formic acid (4 mL), and stirred at room temperature for 3 h. The mixture was then adjusted pH 9 with saturated sodium carbonate solution and extracted with ethyl acetate (20 mL*3). The combined organic layer was washed with water (20 mL*2) and brine (20 mL) successively, dried over anhydrous sodium sulfate, and then concentrated to obtain crude product B-5-1 (130 mg, yield 66%), which was directly used at the next step.

LCMS: ESI-MS(+), [M+1]=384.2.

### Synthesis of ((S)-1-(((S)-1-cyclohexyl-2-(6-(4-fluorobenzyl)-5-oxo-2,3,5,6-tetrahydro-1H-pyrrolo[2,3-c]pyridine-1-yl)-2-oxyethyl)amino)-1-oxopropyl-2-yl)(methyl)tert-butyl carbamate (B-6-1)

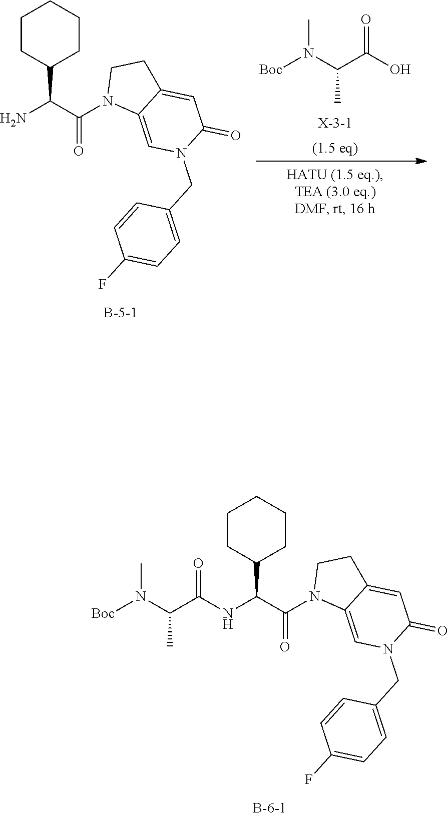

To a suspension of (S)-1-(2-amino-2-cyclohexyl acetyl)-6-(4-fluorobenzyl)-1,2,3,6-tetrahydro-5H-pyrrolo[2,3-c]pyridine-5-one (B-5-1) (130 mg, 0.34 mmol, 1.0 equivalent), N-(tert-butoxycarbonyl)-N-methyl-L-alanine (X-3-1) (104 mg, 0.51 mmol, 1.5 equivalents) and triethylamine (103 mg, 1.02 mmol, 3.0 equivalents) in N,N-dimethylformamide (3 mL) was added 2-(7-azabenzotriazol-1-yl)-N,N,N′,N′-tetramethyluronium hexafluorophosphate (194 mg, 0.51 mmol, 1.5 equivalents), and stirred at room temperature for 16 h. The reaction mixture was diluted with water (20 mL) and extracted with ethyl acetate (20 mL*3). The combined organic layer was washed with water (20 mL*2) and brine (20 mL), successively, dried over anhydrous sodium sulfate and then concentrated. The crude product was purified by silica gel column chromatography (petroleum ether/ethyl acetate=3/1) to obtain B-6-1 (150 mg, yield 77%).

LCMS: ESI-MS(+), [M+1]=569.2.

### Synthesis of (S)-N-((S)-1-cyclohexyl-2-(6-(4-fluorobenzyl)-5-oxo-2,3,5,6-tetrahydro-1H-pyrrolo[2,3-c]pyridine-1-yl)-2-oxyethyl)-2-(methylamino)propanamide (I-2-1)

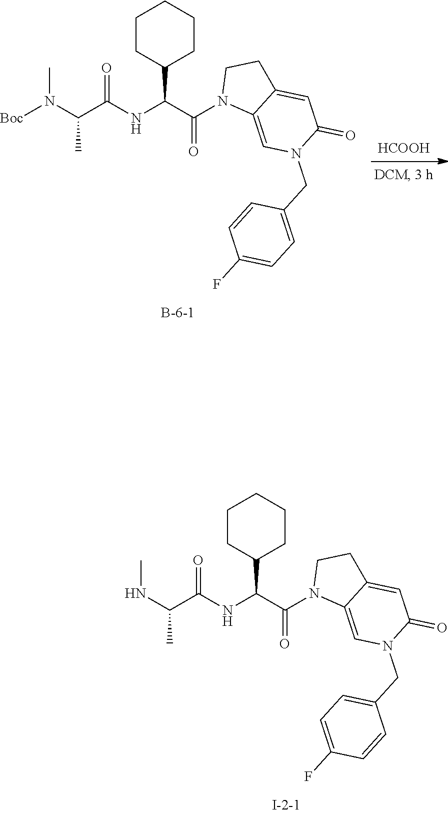 [SMILES: C[C@@H](C(=O)N[C@H](C(=O)N1CCc2cc(=O)n(Cc3ccc(F)cc3)cc21)C1CCCCC1)N(C)C(=O)OC(C)(C)C ; "HCOOH" ; CN[C@@H](C)C(=O)N[C@H](C(=O)N1CCc2cc(=O)n(Cc3ccc(F)cc3)cc21)C1CCCCC1]

To a solution of ((S)-1-(((S)-1-cyclohexyl-2-(6-(4-fluorobenzyl)-5-oxo-2,3,5,6-tetrahydro-1H-pyrrolo[2,3-c]pyridine-1-yl)-2-oxyethyl)amino)-1-oxopropyl-2-yl)(methyl)tert-butyl carbamate (B-6-1) (140 mg, 0.25 mmol, 1.0 equivalent) in dichloromethane (1.5 mL) was added formic acid (6 mL) and stirred at room temperature for 3 h. The reaction mixture was concentrated to obtain a crude product, which was further purified by reverse HPLC preparation to obtain I-2-1 (34.4 mg, yield 27%).

¹H NMR (400 MHz, DMSO-d₆) δ 8.28 (s, 1H), 8.25 (d, J=9.2 Hz, 2H), 7.32 (dd, J=8.4, 5.7 Hz, 2H), 7.16 (t, J=8.8 Hz, 2H), 6.38 (s, 1H), 5.14-5.01 (m, 2H), 4.42 (t, J=7.9 Hz, 1H), 4.18 (dq, J=30.6, 8.5 Hz, 2H), 3.21 (q, J=6.7 Hz, 1H), 3.10 (t, J=7.9 Hz, 2H), 2.25 (s, 3H), 1.63 (ddd, J=50.2, 31.9, 11.2 Hz, 6H), 1.20-0.89 (m, 8H).

LCMS: ESI-MS(+), [M+1]=469.2.

The following compounds are prepared by referring to the preparation method in Embodiment 15:

| Embodiment | No. | ¹H-NMR | MS |
|---|---|---|---|
| Embodiment | I-2-2 | ¹H NMR (400 MHz, DMSO) δ 8.28 (d, | ESI-MS: 455.2 |
| 16 | | J = 5.6 Hz, 3H), 7.32 (dd, J = 8.5, 5.6 | [M + H]⁺ |
| | | Hz, 2H), 7.16 (t, J = 8.8 Hz, 2H), 6.38 | |
| | | (s, 1H), 5.13 (d, J = 14.4 Hz, 1H), 5.01 | |
| | | (d, J = 14.4 Hz, 1H), 4.38 (s, 2H), 4.22- | |
| | | 4.12 (m, 3H), 3.52 (d, J = 7.1 Hz, 1H), | |
| | | 3.11 (d, J = 8.1 Hz, 2H), 1.79-1.49 (m, | |
| | | 6H), 1.21-0.96 (m, 8H). | |
| Embodiment | I-2-3 | ¹H NMR (400 MHz, DMSO-d₆) δ 8.49 | ESI-MS: 470.1 |
| 17 | | (d, J = 2.7 Hz, 1H), 8.24 (s, 1H), 7.91 | [M + H]⁺ |
| | | (d, J = 8.7 Hz, 1H), 7.71 (dd, J = 7.6, | |
| | | 4.3 Hz, 2H), 7.39 (dd, J = 8.6, 4.4 Hz, | |
| | | 1H), 6.40 (d, J = 6.7 Hz, 1H), 5.34 (d, | |
| | | J = 14.9 Hz, 1H), 5.18 (d, J = 14.9 Hz, | |
| | | 1H), 4.24 (s, OH), 4.00-3.91 (m, 3H), | |
| | | 3.09 (q, J = 6.8 Hz, 1H), 3.02-2.91 (m, | |
| | | 1H), 2.85 (dd, J = 9.3, 4.6 Hz, 1H), | |
| | | 2.23 (s, 2H), 1.67-1.38 (m, 3H), 1.10 | |
| | | (d, J = 6.8 Hz, 3H), 1.04-0.85 (m, 3H). | |
| Embodiment | I-2-4 | ¹H NMR (400 MHz, DMSO) δ 8.30 (s, | ESI-MS: 487.1 |
| 18 | | 1H), 8.27-8.11 (m, 2H), 7.34-7.20 (m, | [M + H]⁺ |
| | | 2H), 7.06 (td, J = 8.5, 2.4 Hz, 1H), 6.36 | |
| | | (s, 1H), 5.07 (q, J = 14.8 Hz, 2H), 4.44 | |
| | | (t, J = 8.0 Hz, 1H), 4.19 (dq, J = 30.6, | |
| | | 9.0 Hz, 2H), 3.12 (dt, J = 16.0, 7.5 Hz, | |
| | | 3H), 2.22 (s, 3H), 1.64 (ddd, J = 49.4, | |
| | | 30.6, 12.7 Hz, 6H), 1.21-0.88 (m, 8H). | |
| Embodiment | I-2-5 | ¹H NMR (400 MHz, DMSO) δ 8.38 (s, | ESI-MS: 470.1 |
| 19 | | 1H), 8.32-8.12 (m, 3H), 7.89 (td, J = | [M + H]⁺ |
| | | 8.2, 2.4 Hz, 1H), 7.15 (dd, J = 8.5, 2.7 | |
| | | Hz, 1H), 6.39 (s, 1H), 5.13 (q, J = 14.4 | |
| | | Hz, 2H), 4.43 (d, J = 8.0 Hz, 1H), 4.18 | |
| | | (dq, J = 34.6, 8.9 Hz, 3H), 3.23-3.04 | |
| | | (m, 3H), 2.23 (s, 3H), 1.78-1.50 (m, | |
| | | 6H), 1.20-0.91 (m, 8H). | |
| Embodiment | I-2-6 | ¹H NMR (400 MHz, DMSO) δ 8.31 (s, | ESI-MS: 476.2 |
| 20 | | 1H), 8.26-8.12 (m, 2H), 7.80 (d, J = 8.1 | [M + H]⁺ |
| | | Hz, 2H), 7.40 (d, J = 8.1 Hz, 2H), 6.41 | |
| | | (s, 1H), 5.19 (q, J = 15.2 Hz, 2H), 4.43 | |
| | | (t, J = 8.0 Hz, 1H), 4.28-4.13 (m, 3H), | |
| | | 3.12 (t, J = 8.0 Hz, 3H), 2.22 (s, 3H), | |
| | | 1.64 (ddd, J = 50.2, 31.2, 11.8 Hz, 6H), | |
| | | 1.19-0.90 (m, 8H). | |
| Embodiment | I-2-7 | ¹H NMR (400 MHz, DMSO) δ 8.47 (d, | ESI-MS: 483.1 |
| 21 | | J = 7.9 Hz, 1H), 8.28 (s, 1H), 8.18 (s, | [M + H]⁺ |
| | | 1H), 7.32 (dd, J = 8.5, 5.6 Hz, 2H), | |
| | | 7.16 (t, J = 8.9 Hz, 2H), 6.39 (s, 1H), | |
| | | 5.20-4.95 (m, 2H), 4.40 (t, J = 8.1 Hz, | |
| | | 1H), 4.27 (dd, J = 9.9, 7.5 Hz, 1H), | |
| | | 4.15 (q, J = 8.9 Hz, 1H), 3.14 (dt, J = | |
| | | 34.7, 7.3 Hz, 3H), 2.24 (s, 3H), 1.84- | |
| | | 1.46 (m, 8H), 1.21-0.92 (m, 5H), 0.82 | |
| | | (t, J = 7.4 Hz, 3H). | |
| Embodiment | I-2-8 | ¹H NMR (400 MHz, DMSO) δ 8.36 (d, | ESI-MS: 469.2 |
| 22 | | J = 7.9 Hz, 1H), 8.28 (d, J = 7.9 Hz, | [M + H]⁺ |
| | | 2H), 7.37-7.27 (m, 2H), 7.16 (t, J = 8.7 | |
| | | Hz, 2H), 6.39 (s, 1H), 5.17 (d, J = 14.4 | |
| | | Hz, 1H), 4.98 (d, J = 14.3 Hz, 1H), 4.37 | |
| | | (d, J = 7.9 Hz, 1H), 4.28 (q, J = 9.1 Hz, | |
| | | 1H), 4.14 (q, J = 8.8 Hz, 1H), 3.32 (d, | |
| | | J = 5.8 Hz, 1H), 3.10 (t, J = 7.8 Hz, 2H), | |
| | | 1.80-1.55 (m, 6H), 1.50 (t, J = 10.3 Hz, | |
| | | 2H), 1.25-1.05 (m, 4H), 0.96 (d, J = | |
| | | 11.1 Hz, 1H), 0.83 (t, J = 7.3 Hz, 3H). | |
| Embodiment | I-2-9 | ¹H NMR (400 MHz, DMSO) δ 8.28 (s, | ESI-MS: 483.1 |
| 23 | | 1H), 8.20 (s, 1H), 8.04 (d, J = 8.1 Hz, | [M + H]⁺ |
| | | 1H), 7.32 (dd, J = 8.3, 5.6 Hz, 2H), | |
| | | 7.16 (t, J = 8.7 Hz, 2H), 6.39 (s, 1H), | |
| | | 5.18-4.98 (m, 2H), 4.37 (t, J = 7.7 Hz, | |
| | | 1H), 4.23 (t, J = 8.8 Hz, 1H), 4.14 (t, | |
| | | J = 8.8 Hz, 1H), 3.10 (t, J = 8.1 Hz, 2H), | |
| | | 2.17 (s, 3H), 1.86-1.45 (m, 6H), 1.23- | |
| | | 0.83 (m, 11H). | |
| Embodiment | I-2-10 | ¹H NMR (400 MHz, DMSO) δ 8.28 (s, | ESI-MS: 429.1 |
| 24 | | 1H), 8.18 (d, J = 8.5 Hz, 2H), 7.32 (dd, | [M + H]⁺ |
| | | J = 8.2, 5.7 Hz, 2H), 7.16 (t, J = 8.8 Hz, | |
| | | 2H), 6.39 (s, 1H), 5.08 (q, J = 14.4 Hz, | |
| | | 2H), 4.43 (t, J = 7.8 Hz, 1H), 4.23-4.11 | |
| | | (m, 2H), 3.12 (dt, J = 15.9, 7.2 Hz, 3H), | |
| | | 2.23 (s, 3H), 2.13-1.99 (m, 1H), 1.13 (d, | |
| | | J = 6.7 Hz, 3H), 0.89 (d, J = 6.6 Hz, 6H). | |
| Embodiment | I-2-11 | ¹H NMR (400 MHz, DMSO) δ 8.30 (s, | ESI-MS: 443.3 |
| 25 | | 1H), 8.22 (s, 1H), 8.09 (d, J = 8.2 Hz, | [M + H]⁺ |
| | | 1H), 7.33 (dd, J = 8.3, 5.8 Hz, 2H), | |
| | | 7.16 (t, J = 8.8 Hz, 2H), 6.38 (s, 1H), | |
| | | 5.16 (d, J = 14.3 Hz, 1H), 5.00 (d, J = | |
| | | 14.4 Hz, 1H), 4.53 (d, J = 8.8 Hz, 1H), | |
| | | 4.19 (q, J = 7.8 Hz, 2H), 3.21-3.05 (m, | |
| | | 3H), 2.22 (s, 3H), 1.13 (d, J = 6.6 Hz, | |
| | | 3H), 0.98 (s, 9H). | |
| Embodiment | I-2-12 | ¹H NMR (400 MHz, DMSO) δ 8.30 (s, | ESI-MS: 481.2 |
| 26 | | 1H), 7.39-7.26 (m, 2H), 7.16 (t, J = 8.8 | [M + H]⁺ |
| | | Hz, 2H), 6.38 (s, 1H), 5.07 (q, J = 14.4 | |
| | | Hz, 2H), 4.43 (d, J = 7.0 Hz, 1H), 4.16 | |
| | | (dq, J = 33.1, 8.9 Hz, 2H), 3.32 (s, 3H), | |
| | | 3.10 (t, J = 7.7 Hz, 2H), 1.64 (ddd, J = | |
| | | 43.9, 25.6, 12.7 Hz, 6H), 1.22-0.76 (m, | |
| | | 9H). | |
| Embodiment | I-2-13 | ¹H NMR (400 MHz, DMSO) δ 8.28 (d, | ESI-MS: 497.1 |
| 27 | | J = 4.6 Hz, 1H), 7.31 (dd, J = 8.3, 5.4 | [M + H]⁺ |
| | | Hz, 2H), 7.15 (t, J = 8.5 Hz, 2H), 6.39 | |
| | | (s, 1H), 5.19-4.96 (m, 2H), 4.61 (dd, | |
| | | J = 20.3, 6.0 Hz, 2H), 4.52-4.10 (m, 5H), | |
| | | 3.11 (t, J = 8.0 Hz, 2H), 2.09 (s, 3H), | |
| | | 1.83-1.48 (m, 6H), 1.19-0.82 (m, 5H). | |
| Embodiment | I-2-14 | ¹H NMR (400 MHz, MeOD) δ 8.37 (s, | ESI-MS: 495.2 |
| 28 | | 1H), 7.41-7.29 (m, 2H), 7.05 (t, J = 8.7 | [M + H]⁺ |
| | | Hz, 2H), 6.51 (s, 1H), 5.13 (d, J = 7.8 | |
| | | Hz, 2H), 4.48 (dd, J = 18.4, 9.2 Hz, | |
| | | 2H), 4.23 (d, J = 9.8 Hz, 1H), 3.22 (t, | |
| | | J = 7.9 Hz, 2H), 2.47 (s, 2H), 2.29 (s, | |
| | | 3H), 2.15 (dt, J = 19.9, 8.7 Hz, 2H), | |
| | | 2.00-1.63 (m, 8H), 1.17 (ddd, J = 48.2, | |
| | | 25.2, 13.4 Hz, 5H). | |
| Embodiment | I-2-15 | ¹H NMR (400 MHz, DMSO) δ 8.28 (s, | ESI-MS: 472.2 |
| 29 | | 1H), 8.26-8.19 (m, 2H), 7.32 (dd, J = | [M + H]⁺ |
| | | 8.5, 5.6 Hz, 2H), 7.16 (t, J = 8.8 Hz, | |
| | | 2H), 6.38 (s, 1H), 5.16-4.99 (m, 2H), | |
| | | 4.42 (t, J = 8.0 Hz, 1H), 4.28-4.08 (m, | |
| | | 2H), 3.24-3.06 (m, 3H), 1.63 (ddd, J = | |
| | | 51.9, 32.3, 12.9 Hz, 6H), 1.24-0.91 (m, | |
| | | 8H). | |
| Embodiment | I-2-16 | ¹H NMR (400 MHz, DMSO) δ 8.31 (d, | ESI-MS: 441.1 |
| 30 | | J = 7.4 Hz, 1H), 8.23 (d, J = 9.4 Hz, | [M + H]⁺ |
| | | 2H), 7.38-7.26 (m, 2H), 7.16 (t, J = 8.8 | |
| | | Hz, 2H), 6.39 (s, 1H), 5.08 (s, 2H), 4.64 | |
| | | (q, J = 7.1 Hz, 1H), 4.20-4.11 (m, 2H), | |
| | | 3.13 (dt, J = 16.3, 7.2 Hz, 3H), 2.25 (s, | |
| | | 3H), 1.68-1.47 (m, 2H), 1.15 (d, J = 6.8 | |
| | | Hz, 3H), 0.73 (s, 1H), 0.48-0.30 (m, | |
| | | 2H), 0.09 (dd, J = 17.5, 3.6 Hz, 2H). | |
| Embodiment | I-2-17 | ¹H NMR (400 MHz, DMSO) δ 8.81 (d, | ESI-MS: 483.2 |
| 31 | | J = 8.1 Hz, 3H), 8.01 (s, 1H), 7.33 (dd, | [M + H]⁺ |
| | | J = 8.5, 5.5 Hz, 2H), 7.20 (t, J = 8.8 Hz, | |
| | | 2H), 6.40 (s, 1H), 6.20 (q, J = 6.9 Hz, | |
| | | 1H), 4.43 (t, J = 8.0 Hz, 1H), 4.13 (h, | |
| | | J = 9.9 Hz, 2H), 3.82 (q, J = 6.2 Hz, 1H), | |
| | | 3.09 (s, 2H), 2.47 (s, 3H), 1.66 (dd, J = | |
| | | 35.1, 8.2 Hz, 9H), 1.26 (d, J = 6.9 Hz, | |
| | | 3H), 1.21-0.92 (m, 5H). | |
| Embodiment | I-2-18 | ¹H NMR (400 MHz, DMSO) δ 8.81 (d, | ESI-MS: 483.2 |
| 32 | | J = 8.0 Hz, 3H), 8.04 (s, 1H), 7.31 (dd, | [M + H]⁺ |
| | | J = 8.5, 5.6 Hz, 2H), 7.21 (t, J = 8.8 Hz, | |
| | | 2H), 6.40 (s, 1H), 6.18 (q, J = 6.9 Hz, | |
| | | 1H), 4.43 (t, J = 7.9 Hz, 1H), 4.14 (t, | |
| | | J = 8.1 Hz, 2H), 3.86 (q, J = 6.1 Hz, 1H), | |
| | | 3.10 (t, J = 7.9 Hz, 2H), 2.51 (s, 3H), | |
| | | 1.74-1.45 (m, 9H), 1.33 (d, J = 6.9 Hz, | |
| | | 3H), 1.21-0.94 (m, 5H). | |
| Embodiment | I-2-19 | ¹H NMR (400 MHz, DMSO) δ 8.26 (d, | ESI-MS: 471.1 |
| 33 | | J = 18.7 Hz, 3H), 7.32 (dd, J = 8.2, 5.8 | [M + H]⁺ |
| | | Hz, 2H), 7.16 (t, J = 8.8 Hz, 2H), 6.39 | |
| | | (s, 1H), 5.07 (q, J = 14.4 Hz, 2H), 4.49 | |
| | | (t, J = 7.9 Hz, 1H), 4.19 (t, J = 8.0 Hz, | |
| | | 2H), 3.87-3.78 (m, 3H), 3.29-3.07 (m, | |
| | | 5H), 2.22 (s, 3H), 2.01 (s, 1H), 1.57 (d, | |
| | | J = 11.9 Hz, 1H), 1.41-1.24 (m, 3H), | |
| | | 1.13 (d, J = 6.7 Hz, 3H). | |
| Embodiment | I-2-20 | ¹H NMR (400 MHz, DMSO) δ 9.51 (s, | ESI-MS: 461.2 |
| 34 | | 1H), 8.91 (s, 1H), 8.79 (d, J = 8.1 Hz, | [M + H]⁺ |
| | | 1H), 8.31 (s, 1H), 7.37-7.20 (m, 2H), | |
| | | 7.07 (td, J = 8.5, 2.6 Hz, 1H), 6.37 (s, | |
| | | 1H), 5.16 (d, J = 14.7 Hz, 1H), 5.01 (d, | |
| | | J = 14.7 Hz, 1H), 4.55 (d, J = 8.2 Hz, | |
| | | 1H), 4.19 (dt, J = 27.1, 8.5 Hz, 2H), | |
| | | 3.96 (td, J = 7.3, 4.6 Hz, 1H), 3.11 (t, | |
| | | J = 8.1 Hz, 2H), 2.46 (t, J = 5.2 Hz, 3H), | |
| | | 1.33 (d, J = 6.8 Hz, 3H), 1.02 (s, 9H). | |
| Embodiment | I-2-21 | ¹H NMR (400 MHz, DMSO) δ 8.24 (d, | ESI-MS: 455.2 |
| 35 | | J = 20.5 Hz, 3H), 7.39-7.26 (m, 2H), | [M + H]⁺ |
| | | 7.21-7.08 (m, 2H), 6.38 (s, 1H), 5.16- | |
| | | 5.02 (m, 2H), 4.51 (t, J = 8.3 Hz, 1H), | |
| | | 4.16 (ddd, J = 11.1, 6.7, 2.6 Hz, 2H), | |
| | | 3.21-3.03 (m, 4H), 2.22 (s, 4H), 1.71- | |
| | | 1.39 (m, 6H), 1.35-1.20 (m, 2H), 1.12 | |
| | | (d, J = 6.8 Hz, 3H). | |
| Embodiment | I-2-22 | ¹H NMR (400 MHz, DMSO) δ 8.93- | ESI-MS: 464.1 |
| 36 | | 8.77 (m, 2H), 8.75 (d, J = 8.3 Hz, 1H), | [M + H]⁺ |
| | | 8.31 (s, 1H), 7.36-7.23 (m, 2H), 7.07 | |
| | | (td, J = 8.6, 2.0 Hz, 1H), 6.37 (s, 1H), | |
| | | 5.16 (d, J = 14.7 Hz, 1H), 5.00 (d, J = | |
| | | 14.7 Hz, 1H), 4.56 (d, J = 8.3 Hz, 1H), | |
| | | 4.18 (dq, J = 42.4, 9.7 Hz, 2H), 3.94 | |
| | | (dt, J = 11.9, 5.3 Hz, 1H), 3.11 (t, J = | |
| | | 8.0 Hz, 2H), 1.31 (d, J = 6.9 Hz, 3H), | |
| | | 1.01 (s, 9H). | |
| Embodiment | I-2-23 | ¹H NMR (400 MHz, DMSO) δ 8.40 (s, | ESI-MS: 459.2 |
| 37 | | 1H), 8.04 (d, J = 9.0 Hz, 1H), 7.20 (t, | [M + H]⁺ |
| | | J = 7.6 Hz, 1H), 6.64-6.55 (m, 2H), 6.40 | |
| | | (s, 1H), 5.06-4.89 (m, 2H), 4.55 (d, J = | |
| | | 9.0 Hz, 1H), 4.19 (p, J = 9.9, 8.7 Hz, | |
| | | 2H), 3.10 (t, J = 7.0 Hz, 3H), 2.19 (s, | |
| | | 3H), 1.12 (d, J = 6.8 Hz, 3H), 0.98 (s, | |
| | | 9H). | |
| Embodiment | I-2-24 | ¹H NMR (400 MHz, DMSO) δ 9.43 (s, | ESI-MS: 473.2 |
| 38 | | 1H), 8.89 (s, 1H), 8.76 (d, J = 8.1 Hz, | [M + H]⁺ |
| | | 1H), 8.32 (s, 1H), 7.15-7.08 (m, 1H), | |
| | | 6.94 (dd, J = 11.3, 2.2 Hz, 1H), 6.73 | |
| | | (td, J = 8.5, 2.3 Hz, 1H), 6.34 (s, 1H), | |
| | | 5.02-4.86 (m, 2H), 4.54 (d, J = 8.1 Hz, | |
| | | 1H), 4.19 (dq, J = 30.9, 9.6, 9.0 Hz, | |
| | | 2H), 4.01-3.91 (m, 1H), 3.82 (s, 3H), | |
| | | 3.10 (t, J = 7.8 Hz, 2H), 2.46 (t, J = 5.1 | |
| | | Hz, 3H), 1.32 (d, J = 6.8 Hz, 3H), 1.02 | |
| | | (s, 9H). | |
| Embodiment | I-2-25 | ¹H NMR (400 MHz, DMSO) δ 8.86 (d, | ESI-MS: 497.3 |
| 39 | | J = 7.4 Hz, 2H), 8.28 (s, 1H), 7.42-7.30 | [M + H]⁺ |
| | | (m, 2H), 7.17 (t, J = 8.8 Hz, 2H), 6.44 | |
| | | (s, 1H), 5.14 (d, J = 14.3 Hz, 1H), 5.01 | |
| | | (d, J = 14.3 Hz, 1H), 4.38 (t, J = 7.8 | |
| | | Hz, 1H), 3.96 (s, 2H), 3.85 (s, 1H), 3.51 | |
| | | (s, 3H), 1.85-1.48 (m, 6H), 1.31 (s, 6H), | |
| | | 1.26 (s, 3H), 1.20-0.95 (m, 5H). | |
| Embodiment | I-2-26 | ¹H NMR (400 MHz, DMSO) δ 8.32 (s, | ESI-MS: 489.1 |
| 40 | | 1H), 8.21 (s, 1H), 8.13 (d, J = 8.8 Hz, | [M + H]⁺ |
| | | 1H), 7.36-7.23 (m, 2H), 7.07 (td, J = | |
| | | 8.5, 1.9 Hz, 1H), 6.40 (s, 1H), 5.13 (d, | |
| | | J = 14.7 Hz, 1H), 5.02 (d, J = 14.6 Hz, | |
| | | 1H), 4.52 (d, J = 8.8 Hz, 1H), 4.04 (d, | |
| | | J = 10.3 Hz, 1H), 3.94 (d, J = 10.3 Hz, | |
| | | 1H), 3.22-3.17 (m, 1H), 2.22 (s, 3H), | |
| | | 1.28 (d, J = 5.3 Hz, 6H), 1.13 (d, J = | |
| | | 6.8 Hz, 3H), 1.00 (s, 9H). | |
| Embodiment | I-2-27 | ¹H NMR (400 MHz, MeOD) δ 8.39 (s, | ESI-MS: 515.1 |
| 41 | | 1H), 7.41 (q, J = 8.3 Hz, 1H), 7.03-6.91 | [M + H]⁺ |
| | | (m, 2H), 6.45 (s, 1H), 5.23-5.10 (m, | |
| | | 2H), 4.49 (d, J = 8.7 Hz, 1H), 4.19 (d, | |
| | | J = 10.2 Hz, 1H), 3.97 (d, J = 10.2 Hz, | |
| | | 1H), 3.87 (q, J = 6.9 Hz, 1H), 2.66 (s, | |
| | | 3H), 1.77 (ddd, J = 44.5, 27.7, 12.2 Hz, | |
| | | 6H), 1.47 (d, J = 7.0 Hz, 3H), 1.37 (d, | |
| | | J = 10.1 Hz, 6H), 1.20 (ddd, J = 42.5, | |
| | | 21.1, 10.9 Hz, 5H). | |
| Embodiment | I-2-28 | ¹H NMR (400 MHz, DMSO) δ 8.81 (s, | ESI-MS: 492.2 |
| 42 | | 1H), 8.75 (d, J = 8.2 Hz, 1H), 8.31 (s, | [M + H]⁺ |
| | | 1H), 7.39-7.31 (m, 1H), 7.31-7.24 (m, | |
| | | 1H), 7.08 (td, J = 8.5, 2.1 Hz, 1H), 6.42 | |
| | | (s, 1H), 5.15 (d, J = 14.6 Hz, 1H), 5.00 | |
| | | (d, J = 14.7 Hz, 1H), 4.53 (d, J = 8.1 | |
| | | Hz, 1H), 4.05 (d, J = 10.3 Hz, 1H), 3.95 | |
| | | (d, J = 12.5 Hz, 1H), 3.90 (d, J = 10.3 | |
| | | Hz, 1H), 1.29 (t, J = 7.8 Hz, 9H), 1.02 | |
| | | (s, 9H). | |
| Embodiment | I-2-29 | ¹H NMR (400 MHz, DMSO) δ 8.82 (s, | ESI-MS: 474.2 |
| 43 | | 1H), 8.70 (d, J = 7.9 Hz, 1H), 8.29 (s, | [M + H]⁺ |
| | | 1H), 7.37 (dd, J = 8.4, 5.7 Hz, 2H), | |
| | | 7.17 (t, J = 8.8 Hz, 2H), 6.43 (s, 1H), | |
| | | 5.19 (d, J = 14.2 Hz, 1H), 4.97 (d, J = | |
| | | 14.2 Hz, 1H), 4.51 (d, J = 7.9 Hz, 1H), | |
| | | 4.04 (d, J = 10.3 Hz, 1H), 3.90 (d, J = | |
| | | 10.2 Hz, 2H), 1.31-1.26 (m, 9H), 1.02 | |
| | | (s, 9H). | |
| Embodiment | I-2-30 | ¹H NMR (400 MHz, DMSO-d₆) δ 8.41- | ESI-MS: 517.2 |
| 44 | | 8.21 (m, 3H), 7.29 (dq, J = 17.9, 9.4, | [M + H]⁺ |
| | | 8.9 Hz, 2H), 7.07 (t, J = 8.4 Hz, 1H), | |
| | | 6.41 (s, 1H), 5.07 (q, J = 14.7 Hz, 2H), | |
| | | 4.47 (t, J = 8.0 Hz, 1H), 3.98 (s, 2H), | |
| | | 3.84 (d, J = 9.2 Hz, 2H), 3.25 (t, J = | |
| | | 11.6 Hz, 2H), 3.18-3.10 (m, 1H), 2.21 | |
| | | (s, 3H), 2.00 (d, J = 7.9 Hz, 1H), 1.60 | |
| | | (d, J = 12.2 Hz, 1H), 1.40 (s, 2H), 1.32- | |
| | | 1.23 (m, 8H), 1.12 (d, J = 6.8 Hz, 3H). | |
| Embodiment | I-2-31 | ¹H NMR (400 MHz, DMSO-d₆) δ 8.33 | ESI-MS: 501.1 |
| 45 | | (s, 1H), 8.22 (s, 1H), 8.08 (d, J = 8.7 | [M + H]⁺ |
| | | Hz, 1H), 7.12 (t, J = 7.6 Hz, 1H), 6.94 | |
| | | (d, J = 11.1 Hz, 1H), 6.73 (t, J = 8.4 | |
| | | Hz, 1H), 6.36 (s, 1H), 5.01-4.84 (m, | |
| | | 2H), 4.51 (d, J = 8.7 Hz, 1H), 4.03 (d, | |
| | | J = 10.2 Hz, 1H), 3.93 (d, J = 10.2 Hz, | |
| | | 2H), 3.82 (s, 3H), 3.15 (q, J = 6.2 Hz, | |
| | | 2H), 2.21 (s, 3H), 1.30-1.25 (m, 10H), | |
| | | 1.13 (d, J = 6.7 Hz, 4H), 0.99 (s, 9H). | |
| Embodiment | I-2-32 | ¹H NMR (400 MHz, DMSO-d₆) δ 8.41 | ESI-MS: 487.1 |
| 46 | | (s, 1H), 8.07 (d, J = 8.8 Hz, 1H), 7.25 | [M + H]⁺ |
| | | (t, J = 7.8 Hz, 1H), 6.63-6.57 (m, 2H), | |
| | | 6.45 (s, 1H), 5.05-4.90 (m, 2H), 4.52 | |
| | | (d, J = 8.8 Hz, 1H), 4.03 (d, J = 10.3 | |
| | | Hz, 1H), 3.93 (d, J = 10.3 Hz, 1H), 3.12 | |
| | | (t, J = 6.9 Hz, 1H), 2.21 (s, 3H), 1.27 | |
| | | (d, J = 4.8 Hz, 6H), 1.12 (d, J = 6.8 Hz, | |
| | | 3H), 0.99 (s, 9H). | |
| Embodiment | I-2-33 | ¹H NMR (400 MHz, DMSO-d₆) δ 8.30 | ESI-MS: 471.1 |
| 47 | | (s, 1H), 8.21 (s, 1H), 8.06 (d, J = 8.7 | [M + H]⁺ |
| | | Hz, 1H), 7.36 (dd, J = 8.5, 5.6 Hz, 2H), | |
| | | 7.16 (t, J = 8.9 Hz, 2H), 6.42 (s, 1H), | |
| | | 5.15 (d, J = 14.3 Hz, 1H), 5.01 (d, J = | |
| | | 14.3 Hz, 1H), 4.51 (d, J = 8.7 Hz, 1H), | |
| | | 4.03 (d, J = 10.3 Hz, 1H), 3.93 (d, J = | |
| | | 10.4 Hz, 1H), 3.13 (q, J = 6.8 Hz, 1H), | |
| | | 2.20 (s, 3H), 1.28 (d, J = 5.5 Hz, 6H), | |
| | | 1.12 (d, J = 6.8 Hz, 3H), 0.99 (s, 9H). | |
| Embodiment | I-2-34 | ¹H NMR (400 MHz, DMSO-d₆) δ 8.55 | ESI-MS: 447.1 |
| 48 | | (d, J = 8.1 Hz, 1H), 8.31 (s, 1H), 8.06 | [M + H]⁺ |
| | | (d, J = 5.2 Hz, 3H), 7.36-7.23 (m, 2H), | |
| | | 7.07 (td, J = 8.5, 2.5 Hz, 1H), 6.37 (s, | |
| | | 1H), 5.15 (d, J = 14.7 Hz, 1H), 5.01 (d, | |
| | | J = 14.7 Hz, 1H), 4.51 (d, J = 8.1 Hz, | |
| | | 1H), 4.26-4.12 (m, 2H), 3.98 (p, J = 6.0 | |
| | | Hz, 1H), 3.10 (t, J = 8.1 Hz, 2H), 1.27 | |
| | | (d, J = 6.9 Hz, 3H), 1.01 (s, 9H). | |
| Embodiment | I-2-35 | ¹H NMR (400 MHz, DMSO-d₆) δ 8.93- | ESI-MS: 464.2 |
| 49 | | 8.71 (m, 3H), 8.31 (s, 1H), 7.36-7.23 | [M + H]⁺ |
| | | (m, 2H), 7.07 (td, J = 9.0, 2.5 Hz, 1H), | |
| | | 6.37 (s, 1H), 5.22-4.94 (m, 2H), 4.56 | |
| | | (d, J = 8.3 Hz, 1H), 4.18 (dq, J = 42.7, | |
| | | 8.9 Hz, 2H), 3.98-3.88 (m, 1H), 3.11 (t, | |
| | | J = 8.2 Hz, 2H), 1.31 (d, J = 6.9 Hz, | |
| | | 3H), 1.01 (s, 9H). | |

### Embodiment 50: Synthesis of(S)-N-((S)-1-cyclohexyl-2-(6-(4-fluorobenzyl)-4-methyl-5-oxo-2,3,4,5-tetrahydro-1H-pyrrolo[3,2-b]pyridine-1-yl)-2-oxyethyl)-2-(methylamino)propanamide (I-3-1)

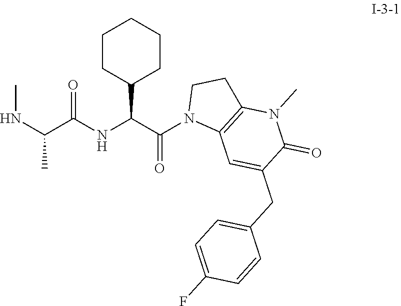 [SMILES: CN[C@@H](C)C(=O)N[C@H](C(=O)N1CCc2c1cc(Cc1ccc(F)cc1)c(=O)n2C)C1CCCCC1]

### Synthesis of 5-methoxy-1H-pyrrolo[3,2-b]pyridine-1-tert-butyl formate (C-2)

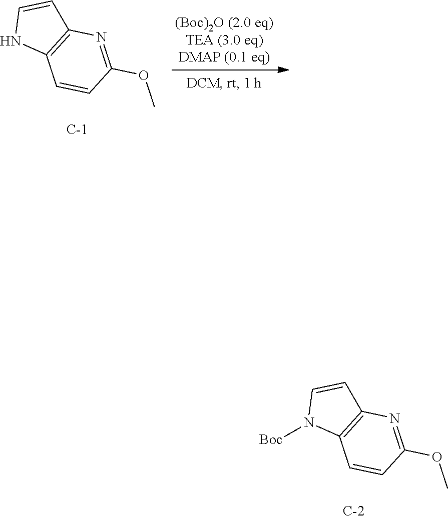

To a solution of 5-methoxy-1H-pyrrolo[3,2-b]pyridine (3.00 g, 20.27 mmol, 1.0 equivalent) in dichloromethane (50 mL) was added triethylamine (6.06 g, 60 mmol, 3.0 equivalents), di-tert-butyl dicarbonate (8.87 g, 40.54 mmol, 2.0 equivalents) and 4-dimethylaminopyridine (244 mg, 2 mmol, 0.1 equivalent), successively, and stirred at room temperature for 1 h. The reaction mixture was then concentrated in vacuo to obtain a crude product, which was purified by a silica gel column (petroleum ether/ethyl acetate=10/1, v/v) to obtain 5-methoxy-1H-pyrrolo[3,2-b]pyridine-1-tert-butyl formate (C-2) (5.30 g, yield 100%).

¹H NMR (400 MHz, DMSO-d₆) δ 8.20 (d, J=8.9 Hz, 1H), 7.84 (d, J=3.7 Hz, 1H), 6.75 (d, J=8.9 Hz, 1H), 6.68 (d, J=3.7 Hz, 1H), 3.88 (s, 3H), 1.62 (s, 9H).

LCMS: ESI-MS(+), [M+1]=249.2.

### Synthesis of 5-methoxy-2,3-dihydro-1H-pyrrolo[3,2-b]pyridine-1-tert-butyl formate (C-3)

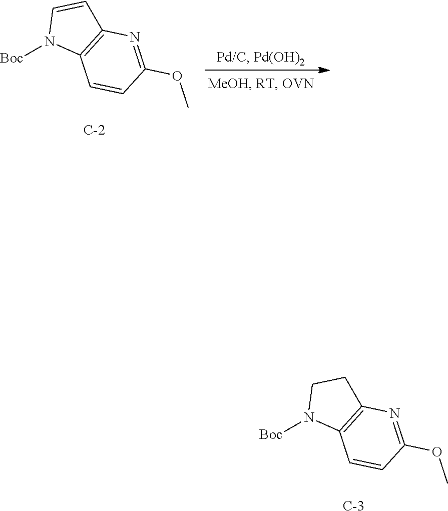

To a solution of 5-methoxy-1H-pyrrolo[3,2-b]pyridine-1-tert-butyl formate (5.30 g, 20.27 mmol, 1.0 equivalent) in methanol (50 mL) was added Pd/C (1.20 g, 10%, w/w, 0.05 equivalent) and palladium hydroxide (1.20 g, 5%, w/w, 0.02 equivalent). The reaction was performed in the atmosphere of hydrogen and stirred at room temperature overnight. The reaction mixture was filtered through a Celite pad. The filtrate was collected and concentrated to give 5-methoxy-2,3-dihydro-1H-pyrrolo[3,2-b]pyridine-1-tert-butyl formate (C-3) (same as above).

¹H NMR (400 MHz, DMSO-d₆) δ 7.72 (d, J=124.9 Hz, 1H), 6.56 (d, J=8.7 Hz, 1H), 3.89 (t, J=8.8 Hz, 2H), 3.78 (s, 3H), 3.06 (t, J=8.8 Hz, 2H), 1.49 (s, 9H).

LCMS: ESI-MS(+), [M+1]=251.2

### Synthesis of 6-bromo-5-methoxy-2,3-dihydro-1H-pyrrolo[3,2-b]pyridine-1-tert-butyl formate (C-4)

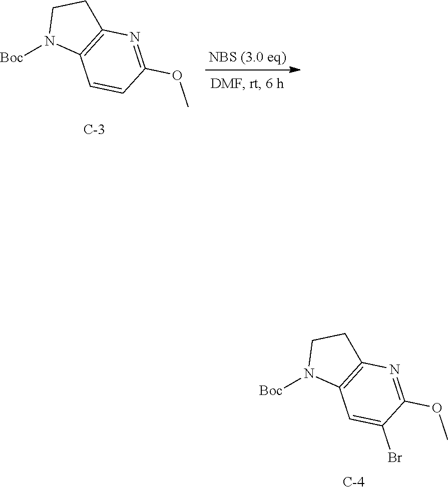

To a solution of 5-methoxy-2,3-dihydro-1H-pyrrolo[3,2-b]pyridine-1-tert-butyl formate (5.10 g, 20.27 mmol, 1.0 equivalent) in N,N-dimethylformamide (60 mL) was added N-bromo-succinimide (NBS) (10.88 g, 61.12 mmol, 3.0 equivalents) and stirred at room temperature for 6 h and. Then water (100 mL) was added, and extracted with ethyl acetate (100 mL*2). The organic layer was washed with water (50 mL*2) and brine (50 mL) successively, dried over anhydrous sodium sulfate and then concentrated under reduced pressure. The crude product was further purified by silica gel column chromatography (petroleum ether/ethyl acetate=30/1) to obtain 6-bromo-5-methoxy-2,3-dihydro-1H-pyrrolo[3,2-b]pyridine-1-tert-butyl formate (C-4) (same as above).

¹H NMR (400 MHz, DMSO-d₆) δ 7.92 (d, J=134.7 Hz, 1H), 3.94 (t, J=8.7 Hz, 2H), 3.87 (s, 3H), 3.08 (t, J=8.7 Hz, 2H), 1.49 (s, 9H).

LCMS: ESI-MS(+), [M+1]=330.2/332.2.

### Synthesis of 5-methoxy-6-(4,4,5,5-tetramethyl-1,3,2-dioxo-borane-2-yl)-2,3-dihydro-1H-pyrrolo[3,2-b]pyridine-1-tert-butyl formate (C-10)

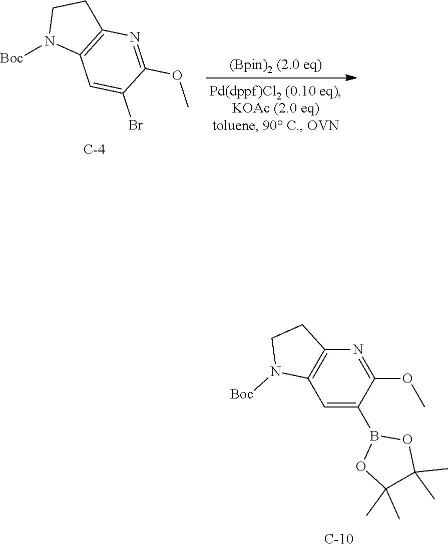

To a solution of 6-bromo-5-methoxy-2,3-dihydro-1H-pyrrolo[3,2-b]pyridine-1-tert-butyl formate (1.20 g, 3.64 mmol, 1.0 equivalent) in 1,4-dioxane (20 mL) was added bisdiboron (1.85 g, 7.28 mmol, 2.0 equivalents), potassium acetate (715 mg, 7.28 mmol, 2.0 equivalents) and Pd(dppf)Cl₂ (267 mg, 0.36 mmol, 0.1 equivalent), successively, and stirred at 90° C. for 16 h. The reaction mixture was diluted with water (50 mL) and extracted with ethyl acetate (50 mL*2). The organic layer was dried over anhydrous sodium sulfate and then concentrated under reduced pressure. The crude product was further purified by silica gel column chromatography (petroleum ether/ethyl acetate=5/1) to obtain 5-methoxy-6-(4,4,5,5-tetramethyl-1,3,2-dioxo-borane-2-yl)-2,3-dihydro-1H-pyrrolo[3,2-b]pyridine-1-tert-butyl formate (C-10) (920 mg, yield 67%).

¹H NMR (400 MHz, DMSO-d₆) δ 8.22-7.89 (m, 1H), 3.90 (t, J=8.2 Hz, 2H), 3.79 (s, 3H), 3.10 (s, 2H), 1.48 (s, 9H), 1.21 (d, J=44.9 Hz, 12H).

LCMS: ESI-MS(+), [M+1−82]=295.1.

### Synthesis of 6-(4-fluorobenzyl)-5-methoxy-2,3-dihydro-1H-pyrrolo[3,2-b]pyridine-1-tert-butyl formate (C-5-1)

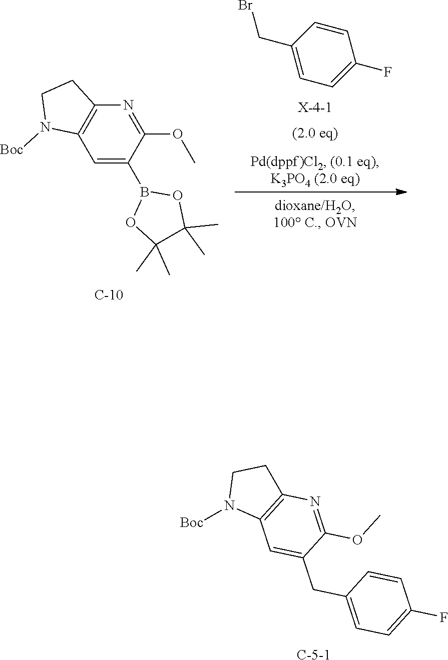

To a solution of 5-methoxy-6-(4,4,5,5-tetramethyl-1,3,2-dioxo-borane-2-yl)-2,3-dihydro-1H-pyrrolo[3,2-b]pyridine-1-tert-butyl formate (920 mg, 2.44 mmol, 1.0 equivalent) in 1,4-dioxane and water (v/v=5/1, 6 mL) was added 4-fluorobenzyl bromide (920 mg, 4.89 mmol, 2.0 equivalents), potassium phosphate (866 mg, 4.89 mmol, 2.0 equivalents) and Pd(dppf)Cl₂ (180 mg, 0.24 mmol, 0.1 equivalent), and stirred at 110° C. overnight. The reaction mixture was diluted with water (20 mL) and extracted with ethyl acetate (20 mL*2). The organic layer was dried over anhydrous sodium sulfate and then concentrated under reduced pressure. The crude product was further purified by silica gel column chromatography (petroleum ether/ethyl acetate=5/1) to obtain 6-(4-fluorobenzyl)-5-methoxy-2,3-dihydro-1H-pyrrolo[3,2-b]pyridine-1-tert-butyl formate (C-5-1) (440 mg, yield 50%).

¹H NMR (400 MHz, DMSO-d₆) δ 7.80 (s, 0.4H) 7.22 (t, J=6.9 Hz, 2.6H), 7.10 (s, 2H), 3.93-3.72 (m, 7H), 3.05 (t, J=8.8 Hz, 2H), 1.40 (d, J=42.0 Hz, 9H).

LCMS: ESI-MS(+), [M+1]=359.4.

### Synthesis of 6-(4-fluorobenzyl)-5-oxo-2,3,4,5-tetrahydro-1H-pyrrolo[3,2-b]pyridine-1-tert-butyl formate (C-6-1)

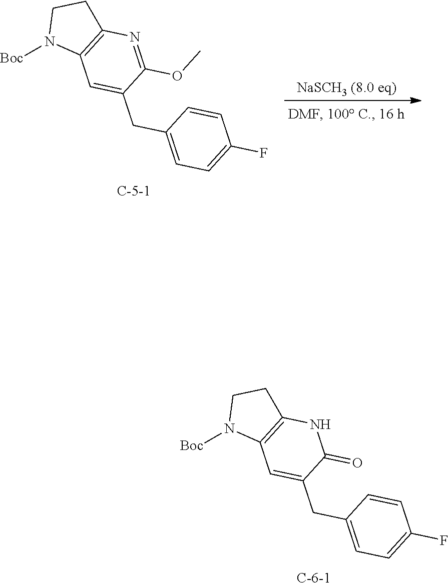 [SMILES: COc1nc2c(cc1Cc1ccc(F)cc1)N(C(=O)OC(C)(C)C)CC2 ; "NaSCH3 (8.0 eq)" ; "C-5-1" ; CC(C)(C)OC(=O)N1CCc2[nH]c(=O)c(Cc3ccc(F)cc3)cc21 ; "C-6-1"]

To a solution of 6-(4-fluorobenzyl)-5-methoxy-2,3-dihydro-1H-pyrrolo[3,2-b]pyridine-1-tert-butyl formate (686 mg, 1.92 mmol, 1.0 equivalent) in N,N-dimethylformamide (6 mL) was added sodium methylmercaptan (1.07 g, 15.36 mmol, 8.0 equivalents), and stirred at 100° C. for 16 h. The reaction mixture was diluted with water (10 mL) and extracted with ethyl acetate (10 mL*3). The organic layer was washed with water (20 mL*2) and brine (20 mL), successively, dried over anhydrous sodium sulfate and then concentrated under reduced pressure. The crude product was further purified by silica gel column chromatography (petroleum ether/ethyl acetate=1/1) to obtain 6-(4-fluorobenzyl)-5-oxo-2,3,4,5-tetrahydro-1H-pyrrolo[3,2-b]pyridine-1-tert-butyl formate (C-6-1) (320 mg, yield 48%).

LCMS: ESI-MS(+), [M+1]=345.2.

### Synthesis of 6-(4-fluorobenzyl)-4-methyl-5-oxo-2,3,4,5-tetrahydro-1H-pyrrolo[3,2-b]pyridine-1-tert-butyl formate (C-7-1)

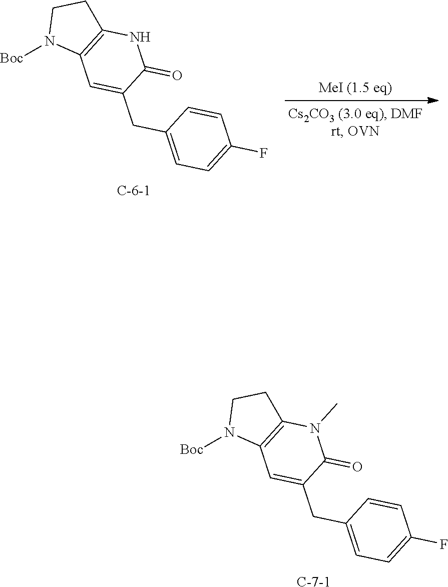 [SMILES: CC(C)(C)OC(=O)N1CCc2[nH]c(=O)c(Cc3ccc(F)cc3)cc21 ; "MeI (1.5 eq)" ; "C-6-1" ; Cn1c2c(cc(Cc3ccc(F)cc3)c1=O)N(C(=O)OC(C)(C)C)CC2 ; "C-7-1"]

To a solution of 6-(4-fluorobenzyl)-5-oxo-2,3,4,5-tetrahydro-1H-pyrrolo[3,2-b]pyridine-1-tert-butyl formate (470 mg, 1.37 mmol, 1.0 equivalent) in N,N-dimethylformamide (10 mL) was added cesium carbonate (1.34 g, 4.11 mmol, 3.0 equivalents). The mixture was kept at 0° C. and then added methyl iodide (291 mg, 2.05 mmol, 1.50 equivalents) slowly. After addition, the system was warmed to room temperature overnight. The reaction mixture was diluted with water (20 mL) and extracted with ethyl acetate (20 mL*3). The organic layer was washed with sodium bisulfite solution (10 mL), water (30 mL*2) and brine (50 mL), successively, dried over anhydrous sodium sulfate and then concentrated under reduced pressure. The crude product was further purified by silica gel column chromatography (petroleum ether/ethyl acetate=2/1, v/v) to obtain 6-(4-fluorobenzyl)-4-methyl-5-oxo-2,3,4,5-tetrahydro-1H-pyrrolo[3,2-b]pyridine-1-tert-butyl formate (C-7-1) (173 mg, yield 35%).

¹H NMR (400 MHz, DMSO-d₆) δ 7.26 (dd, J=8.2, 5.8 Hz, 2H), 7.09 (s, 2H), 3.85 (s, 2H), 3.73 (s, 2H), 3.38 (s, 3H), 3.14 (t, J=8.7 Hz, 2H), 1.37 (d, J=48.0 Hz, 9H).

LCMS: ESI-MS(+), [M+1]=359.2.

### Synthesis of 6-(4-fluorobenzyl)-4-methyl-1,2,3,4-tetrahydro-5H-pyrrolo[3,2-b]pyridine-5-one (C-8-1)

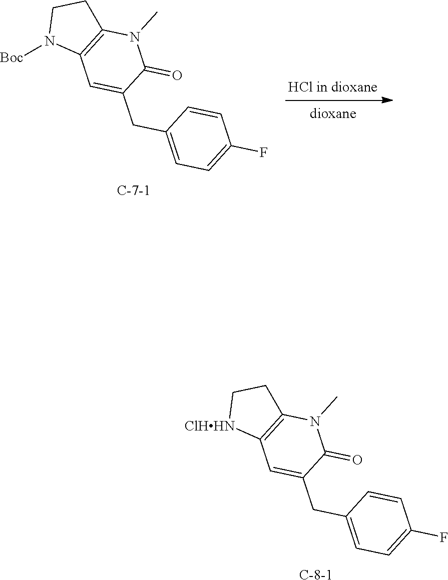

To a solution of 6-(4-fluorobenzyl)-4-methyl-5-oxo-2,3,4,5-tetrahydro-1H-pyrrolo[3,2-b]pyridine-1-tert-butyl formate (173 mg, 0.48 mmol, 1.0 equivalent) in 1,4-dioxane (0.5 mL) was added hydrochloric acid solution (2 mL, 4 mol/L in 1,4-dioxane), and stirred at room temperature for 3 h. The reaction mixture was concentrated in vacuo to obtain the crude product of 6-(4-fluorobenzyl)-4-methyl-1,2,3,4-tetrahydro-5H-pyrrolo[3,2-b]pyridine-5-one hydrochloride (C-8-1) (120 mg, yield 100%), which was directly used at the next step.

LCMS: ESI-MS(+), [M+1]=259.2.

### Synthesis of ((S)-1-(((S)-1-cyclohexyl-2-(6-(4-fluorobenzyl)-4-methyl-5-oxo-2,3,4,5-tetrahydro-1H-pyrrolo[3,2-b]pyridine-1-yl)-2-oxyethyl)amino)-1-oxopropyl-2-yl)(methyl)tert-butyl carbamate (C-9-1)

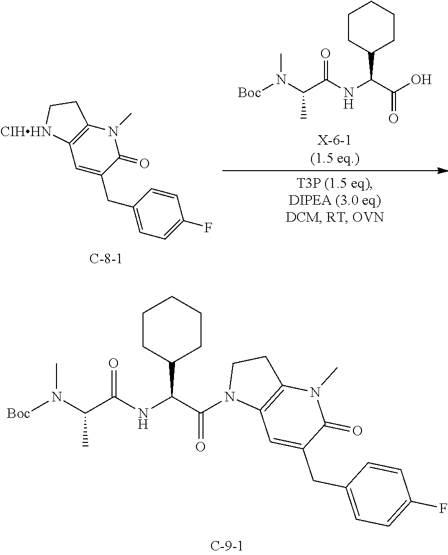

To a solution of 6-(4-fluorobenzyl)-4-methyl-1,2,3,4-tetrahydro-5H-pyrrolo[3,2-b]pyridine-5-one hydrochloride (100 mg, 0.34 mmol, 1.0 equivalent) in dichloromethane (3 mL) was added (S)-2-(S)-2-(tert-butoxycarbonyl)(methyl)amino)propanamide)-2-cyclohexylacetic acid (175 mg, 0.51 mmol, 1.5 equivalents) and N,N-diisopropylethylamine (132 mg, 1.02 mmol, 3.0 equivalents). The mixture was cooled to 0° C. and then added 1-propyl phosphoric anhydride (163 mg, 0.51 mmol, 1.5 equivalents) slowly. After the addition, the system was warmed to room temperature and stirred overnight. The reaction mixture was diluted with water (15 mL) and extracted with ethyl acetate (15 mL*3). The organic layer was dried over anhydrous sodium sulfate and then concentrated under reduced pressure. The crude product was further purified by silica gel column chromatography to obtain ((S)-1-(((S)-1-cyclohexyl-2-(6-(4-fluorobenzyl)-4-methyl-5-oxo-2,3,4,5-tetrahydro-1H-pyrrolo[3,2-b]pyridine-1-yl)-2-oxyethyl)amino)-1-oxopropyl-2-yl)(methyl)tert-butyl carbamate (C-9-1) (150 mg, yield 75%).

LCMS: ESI-MS(+), [M+1]=583.4.

### Synthesis of (S)-N-((S)-1-cyclohexyl-2-(6-(4-fluorobenzyl)-4-methyl-5-oxo-2,3,4,5-tetrahydro-1H-pyrrolo[3,2-b]pyridine-1-yl)-2-oxyethyl)-2-(methylamino)propanamide (I-3-1)

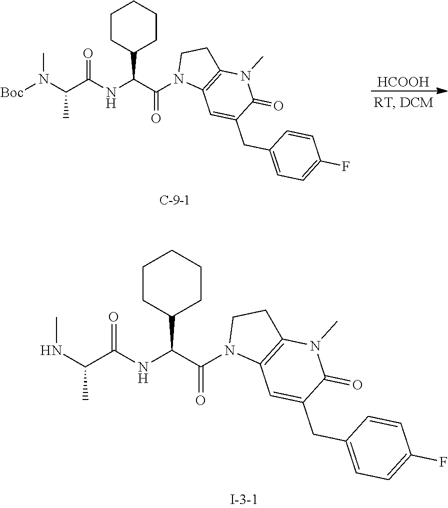 [SMILES: C[C@@H](C(=O)N[C@H](C(=O)N1CCc2c1cc(Cc1ccc(F)cc1)c(=O)n2C)C1CCCCC1)N(C)C(=O)OC(C)(C)C ; "HCOOH" ; CN[C@@H](C)C(=O)N[C@H](C(=O)N1CCc2c1cc(Cc1ccc(F)cc1)c(=O)n2C)C1CCCCC1]

To a solution of ((S)-1-(((S)-1-cyclohexyl-2-(6-(4-fluorobenzyl)-4-methyl-5-oxo-2,3,4,5-tetrahydro-1H-pyrrolo[3,2-b]pyridine-1-yl)-2-oxyethyl)amino)-1-oxopropyl-2-yl)(methyl)tert-butyl carbamate (C-9-1) (150 mg, 0.26 mmol, 1.0 equivalent) in dichloromethane (0.5 mL) was added formic acid (2 mL), and stirred at room temperature for 3 h. The mixture was then concentrated in vacuo to obtain the crude product, which was further purified by reversed phase preparative liquid chromatography to obtain (S)-N-((S)-1-cyclohexyl-2-(6-(4-fluorobenzyl)-4-methyl-5-oxo-2,3,4,5-tetrahydro-1H-pyrrolo[3,2-b]pyridine-1-yl)-2-oxyethyl)-2-(methylamino)propanamide (I-3-1) (57.5 mg, yield 45%).

¹H NMR (400 MHz, DMSO-d₆) δ8.31 (s, 1H), 8.16 (d, J=13.1 Hz, 1H), 7.26 (dd, J=8.3, 5.8 Hz, 2H), 7.07 (t, J=8.8 Hz, 2H), 4.40-4.15 (i, 4H), 3.73 (s, 2H), 3.40 (s, 3H), 3.27 (d, J=7.4 Hz, 2H), 2.28 (s, 3H), 1.58 (ddd, J=53.3, 30.9, 7.2 Hz, 8H), 1.12 (d, J=31.9 Hz, 6H).

LCMS: ESI-MS(+), [M+1]=483.3.

The following compounds were prepared by referring to the preparation method in Embodiment 50:

| Embodiment | No. | ¹H-NMR | MS |
|---|---|---|---|
| Embodiment | I-3-2 | ¹H NMR (400 MHz, DMSO) δ 8.25 (s, | ESI-MS: 457.3 |
| 51 | | 1H), 8.16 (s, 1H), 8.09 (d, J = 9.0 Hz, | [M + H]⁺ |
| | | 1H), 7.29-7.23 (m, 2H), 7.07 (t, J = 8.7 | |
| | | Hz, 2H), 4.49 (d, J = 9.0 Hz, 1H), 4.24 (t, | |
| | | J = 8.3 Hz, 2H), 3.74-3.72 (m, 3H), 3.39 | |
| | | (s, 3H), 3.30-3.22 (m, 2H), 3.21-3.16 (m, | |
| | | 1H), 2.22 (s, 3H), 1.14 (d, J = 6.6 Hz, | |
| | | 3H), 0.98 (s, 9H). | |
| Embodiment | I-3-3 | ¹H NMR (400 MHz, MeOD) δ 8.54 (s, | ESI-MS: 475.3 |
| 52 | | 1H), 8.23 (s, 1H), 7.35-7.25 (m, 1H), | [M + H]⁺ |
| | | 6.95-6.81 (m, 2H), 4.60 (s, 2H), 4.56 (s, | |
| | | 1H), 4.46 (dd, J = 9.8, 7.1 Hz, 1H), 4.31 | |
| | | (td, J = 10.0, 7.2 Hz, 1H), 3.83 (d, J = 2.8 | |
| | | Hz, 1H), 3.62-3.57 (m, 1H), 3.54 (s, 3H), | |
| | | 3.37-3.34 (m, 1H), 2.50 (s, 3H), 1.36 (d, | |
| | | J = 6.9 Hz, 3H), 1.08 (s, 9H). | |
| Embodiment | I-3-4 | ¹H NMR (400 MHz, Methanol-d₄) δ 8.23 | ESI-MS: 478.4 |
| 53 | | (s, 1H), 7.30 (q, J = 8.1 Hz, 1H), 6.88 (q, | [M + H]⁺ |
| | | J= 9.2, 8.6 Hz, 2H), 4.60 (s, 1H), 4.47 (d, | |
| | | J = 8.0 Hz, 1H), 4.32 (t, J = 9.2 Hz, 1H), | |
| | | 3.83 (s, 2H), 3.65 (d, J = 7.1 Hz, 1H), 3.35 | |
| | | (d, J = 8.5 Hz, 2H), 2.52 (s, 3H), 1.37 (d, | |
| | | J = 6.9 Hz, 3H), 1.08 (s, 9H). | |
| Embodiment | I-3-5 | ¹H NMR (400 MHz, DMSO-d₆) δ 8.15 (d, | ESI-MS: 471.4 |
| 54 | | J = 8.1 Hz, 1H), 7.84 (d, J = 9.3 Hz, 1H), | [M + H]⁺ |
| | | 7.30-7.23 (m, 2H), 7.11-7.04 (m, 2H), | |
| | | 4.54-4.45 (m, 1H), 4.24 (t, J = 8.6 Hz, | |
| | | 2H), 3.79-3.68 (m, 2H), 3.39 (s, 3H), 3.26 | |
| | | (d, J = 7.5 Hz, 2H), 3.16 (d, J = 7.2 Hz, | |
| | | 1H), 2.23 (s, 6H), 1.08 (d, J = 6.9 Hz, | |
| | | 3H), 0.98 (s, 9H). | |
| Embodiment | I-3-6 | ¹H NMR (400 MHz, DMSO-d₆) δ 8.22 (d, | ESI-MS: 489.4 |
| 55 | | J = 9.0 Hz, 1H), 8.08 (s, 1H), 7.33 (td, J = | [M + H]⁺ |
| | | 8.7, 6.7 Hz, 1H), 7.18 (td, J = 9.9, 2.6 Hz, | |
| | | 1H), 7.01 (td, J = 8.6, 2.7 Hz, 1H), 4.54- | |
| | | 4.49 (m, 1H), 4.24 (ddd, J = 10.2, 7.0, 4.2 | |
| | | Hz, 2H), 3.90 (q, J = 7.0 Hz, 2H), 3.73 (d, | |
| | | J = 4.9 Hz, 2H), 3.34 (d, J = 3.6 Hz, 1H), | |
| | | 3.33-3.27 (m, 2H), 2.27 (s, 3H), 1.18 (q, | |
| | | J = 6.9 Hz, 6H), 0.97 (s, 9H). | |
| Embodiment | I-3-7 | ¹H NMR (400 MHz, Methanol-d₄) δ 8.49 | ESI-MS: 503.3 |
| 56 | | (s, 1H), 8.23 (s, 1H), 7.31 (q, J = 8.1 Hz, | [M + H]⁺ |
| | | 1H), 6.95-6.83 (m, 2H), 4.58 (s, 1H), 4.11 | |
| | | (dd, J = 71.6, 10.4 Hz, 2H), 3.80 (dd, J = | |
| | | 16.6, 4.7 Hz, 3H), 3.65 (s, 3H), 2.59 (s, | |
| | | 3H), 1.51 (d, J = 10.5 Hz, 6H), 1.41 (d, | |
| | | J = 6.8 Hz, 3H), 1.09 (s, 9H). | |
| Embodiment | I-3-8 | ¹H NMR (400 MHz, DMSO-d₆) δ 8.20 (d, | ESI-MS: 543.3 |
| 57 | | J = 16.7 Hz, 2H), 8.12 (d, J = 9.0 Hz, 1H), | |
| | | 7.34 (td, J = 8.7, 6.6 Hz, 1H), 7.20 (td, J = | |
| | | 9.9, 2.6 Hz, 1H), 7.02 (td, J = 8.5, 2.6 Hz, | |
| | | 1H), 4.87 (q, J = 9.2 Hz, 2H), 4.48 (d, J = | |
| | | 9.0 Hz, 1H), 4.26 (t, J = 8.7 Hz, 2H), 3.75 | |
| | | (s, 2H), 3.29 (t, J = 7.4 Hz, 2H), 3.17 (t, | |
| | | J = 6.8 Hz, 1H), 2.21 (s, 3H), 1.13 (d, J = | |
| | | 6.8 Hz, 3H), 0.97 (s, 9H). | |
| Embodiment | I-3-9 | ¹H NMR (400 MHz, Methanol-d4) δ 8.48 | ESI-MS: 466.2 |
| 58 | | (s, 1H), 7.13 (d, J = 7.3 Hz, 1H), 6.98 (t, | [M + H]⁺ |
| | | J = 7.7 Hz, 1H), 6.69 (t, J = 7.4 Hz, 1H), | |
| | | 6.51 (d, J = 8.0 Hz, 1H), 4.63 (s, 1H), 4.52 | |
| | | (td, J = 9.7, 6.9 Hz, 1H), 4.36 (td, J = | |
| | | 10.1, 7.5 Hz, 1H), 3.93-3.78 (m, 3H), 3.59 | |
| | | (s, 3H), 3.38 (ddd, J = 10.2, 7.1, 3.1 Hz, | |
| | | 2H), 3.10 (t, J = 8.4 Hz, 2H), 2.60 (s, 3H), | |
| | | 1.43 (d, J = 6.9 Hz, 3H), 1.11 (s, 9H). | |

### Embodiment 59: Synthesis of (S)-N-((S)-1-cyclohexyl-2-(6-(4-fluorobenzyl)-5-oxo-2,3,4,5-tetrahydro-1H-pyrrolo[3,2-b]pyridine-1-yl)-2-oxyethyl)-2-(methylamino)propanamide (I-4-1)

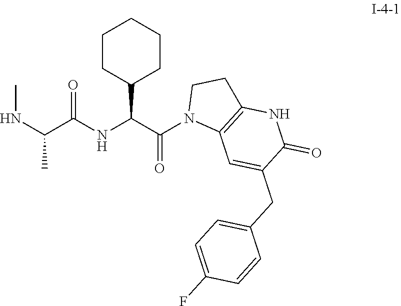 [SMILES: CN[C@@H](C)C(=O)N[C@H](C(=O)N1CCc2[nH]c(=O)c(Cc3ccc(F)cc3)cc21)C1CCCCC1]

### Synthesis of 6-(4-fluorobenzyl)-5-methoxy-2,3-dihydro-1H-pyrrolo[3,2-b]pyridine (C-11-1)

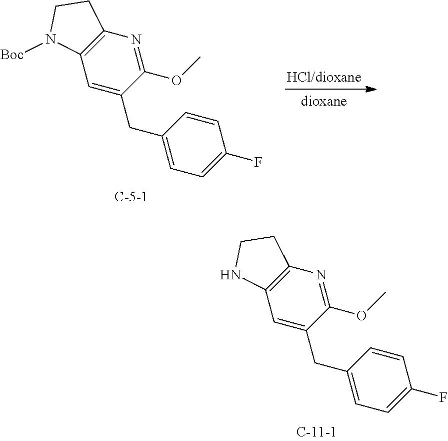 [SMILES: COc1nc2c(cc1Cc1ccc(F)cc1)N(C(=O)OC(C)(C)C)CC2 ; COc1nc2c(cc1Cc1ccc(F)cc1)NCC2]

To a solution of 6-(4-fluorobenzyl)-5-methoxy-2,3-dihydro-1H-pyrrolo[3,2-b]pyridine-1-tert-butyl formate (C-5-1) (120 mg, 0.33 mmol, 1.0 equivalent) in 1,4-dioxane (0.5 mL) was added hydrochloric acid solution (2 mL, 4 mol/L in 1,4-dioxane), and stirred at room temperature for 4 h. The mixture was concentrated in vacuo to obtain the crude product (86 mg, yield 100%) of 6-(4-fluorobenzyl)-5-methoxy-2,3-dihydro-1H-pyrrolo[3,2-b]pyridine hydrochloride (C-11-1).

LCMS: ESI-MS(+), [M+1]=259.2.

### Synthesis of (S)-(1-cyclohexyl-2-(6-(4-fluorobenzyl)-5-methoxy-2,3-dihydro-1H-pyrrolo[3,2-b]pyridine-1-yl)-2-oxyethyl)tert-butyl carbamate (C-12-1)

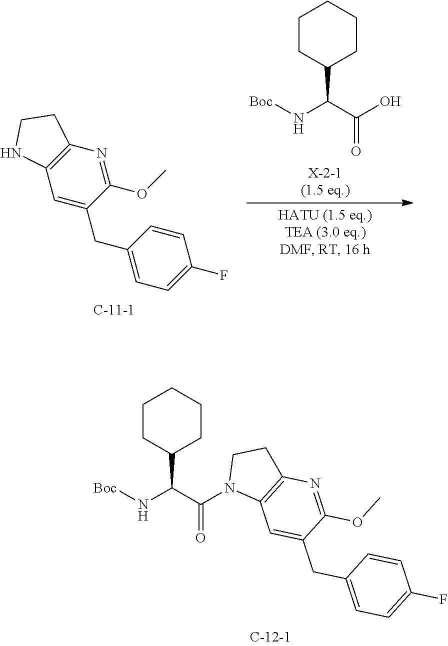 [SMILES: COc1nc2c(cc1Cc1ccc(F)cc1)NCC2 ; CC(C)(C)OC(=O)N[C@H](C(=O)O)C1CCCCC1 ; COc1nc2c(cc1Cc1ccc(F)cc1)N(C(=O)[C@@H](NC(=O)OC(C)(C)C)C1CCCCC1)CC2]

To a solution of 6-(4-fluorobenzyl)-5-methoxy-2,3-dihydro-1H-pyrrolo[3,2-b]pyridine (86 mg, 0.33 mmol, 1.0 equivalent) in N,N-dimethylformamide (3 mL) was added (S)-2-((tert-butoxycarbonyl)amino)-2-cyclohexylacetic acid (128 mg, 0.50 mmol, 1.5 equivalents), triethylamine (101 mg, 1.00 mmol, 3.0 equivalents) and 2-(7-azabenzotriazol-1-yl)-N,N,N′,N′-tetramethyluronium hexafluorophosphate (190 mg, 0.50 mmol, 1.5 equivalents) and stirred at room temperature overnight. The reaction mixture was diluted with water (15 mL) and extracted with ethyl acetate (15 mL*3). The organic layer was washed with water (20 mL*2) and brine (10 mL), successively, dried over anhydrous sodium sulfate and then concentrated under reduced pressure. The crude product was further purified by silica gel column chromatography (petroleum ether/ethyl acetate=3/1,v/v) to obtain (S)-(1-cyclohexyl-2-(6-(4-fluorobenzyl)-5-methoxy-2,3-dihydro-1H-pyrrolo[3,2-b]pyridine-1-yl)-2-oxyethyl)tert-butyl carbamate (C-12-1) (167 mg, yield 100%).

LCMS: ESI-MS(+), [M+1]=498.4.

### Synthesis of (S)-2-amino-2-cyclohexyl-1-(6-(4-fluorobenzyl)-5-methoxy-2,3-dihydro-1H-pyrrolo[3,2-b]pyridine-1-yl)ethane-1-one (C-13-1)

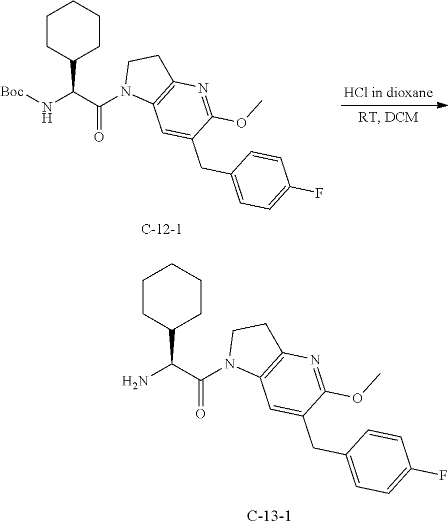 [SMILES: COc1nc2c(cc1Cc1ccc(F)cc1)N(C(=O)[C@@H](NC(=O)OC(C)(C)C)C1CCCCC1)CC2 ; COc1nc2c(cc1Cc1ccc(F)cc1)N(C(=O)[C@@H](N)C1CCCCC1)CC2]

To a solution of (S)-(1-cyclohexyl-2-(6-(4-fluorobenzyl)-5-methoxy-2,3-dihydro-1H-pyrrolo[3,2-b]pyridine-1-yl)-2-oxyethyl)tert-butyl carbamate (167 mg, 0.33 mmol, 1.0 equivalent) in dichloromethane (0.5 mL) was added hydrochloric acid solution (2 mL, 4 mol/L in 1,4-dioxane), and stirred at room temperature for 3 h. The mixture was concentrated to obtain the crude product (132 mg, yield 100%) of (S)-2-amino-2-cyclohexyl-1-(6-(4-fluorobenzyl)-5-methoxy-2,3-dihydro-1H-pyrrolo[3,2-b]pyridine-1-yl)ethane-1-one hydrochloride (C-13-1).

LCMS: ESI-MS(+), [M+1]=398.4.

### Synthesis of ((S)-1-(((S)-1-cyclohexyl-2-(6-(4-fluorobenzyl)-5-methoxy-2,3-dihydro-1H-pyrrolo[3,2-b]pyridine-1-yl)-2-oxyethyl)amino)-1-oxopropyl-2-yl)(methyl)tert-butyl carbamate (C-14-1)

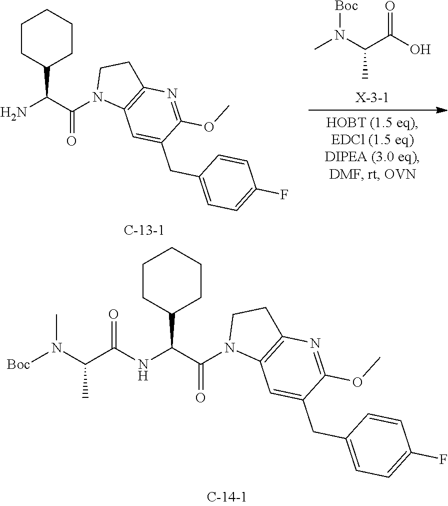 [SMILES: COc1nc2c(cc1Cc1ccc(F)cc1)N(C(=O)[C@@H](N)C1CCCCC1)CC2 ; C[C@@H](C(=O)O)N(C)C(=O)OC(C)(C)C ; COc1nc2c(cc1Cc1ccc(F)cc1)N(C(=O)[C@@H](NC(=O)[C@H](C)N(C)C(=O)OC(C)(C)C)C1CCCCC1)CC2]

To a solution of (S)-2-amino-2-cyclohexyl-1-(6-(4-fluorobenzyl)-5-methoxy-2,3-dihydro-1H-pyrrolo[3,2-b]pyridine-1-yl)ethane-1-one (132 mg, 0.33 mmol, 1.0 equivalent) in N,N-dimethylformamide (4 mL) was added N-(tert-butoxycarbonyl)-N-methyl-L-alanine (101 mg, 0.50 mmol, 1.5 equivalents), N,N-diisopropylethylamine (129 mg, 1.00 mmol, 3.0 equivalents), 1-hydroxybenzotriazole (HOBT) (67.5 mg, 0.50 mmol, 1.5 equivalents) and 1-(3-dimethylaminopropyl)-3-ethyl-carbodiimid monohydrochloride (96.5 mg, 0.50 mmol, 1.5 equivalents), successively, and stirred at room temperature overnight. The reaction mixture was diluted with water (20 mL) and extracted with ethyl acetate (20 mL*3). The organic layer was washed with water (20 mL*2) and brine (20 mL), dried over anhydrous sodium sulfate and then concentrated under reduced pressure. The crude product was further purified by silica gel column chromatography (petroleum ether/ethyl acetate=2/1) to obtain ((S)-1-(((S)-1-cyclohexyl-2-(6-(4-fluorobenzyl)-5-methoxy-2,3-dihydro-1H-pyrrolo[3,2-b]pyridine-1-yl)-2-oxyethyl)amino)-1-oxopropyl-2-yl)(methyl)tert-butyl carbamate (C-14-1) (117 mg, yield 60%).

LCMS: ESI-MS(+), [M+1]=583.5.

### Synthesis of (S)-N-((S)-1-cyclohexyl-2-(6-(4-fluorobenzyl)-5-methoxy-2,3-dihydro-1H-pyrrolo[3,2-b]pyridine-1-yl)-2-oxyethyl)-2-(methylamino)propanamide (C-15-1)

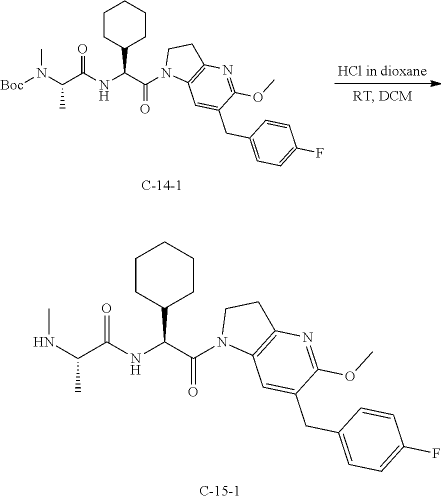 [SMILES: COc1nc2c(cc1Cc1ccc(F)cc1)N(C(=O)[C@@H](NC(=O)[C@H](C)N(C)C(=O)OC(C)(C)C)C1CCCCC1)CC2 ; CN[C@@H](C)C(=O)N[C@H](C(=O)N1CCc2nc(OC)c(Cc3ccc(F)cc3)cc21)C1CCCCC1]

To a solution of ((S)-1-(((S)-1-cyclohexyl-2-(6-(4-fluorobenzyl)-5-methoxy-2,3-dihydro-1H-pyrrolo[3,2-b]pyridine-1-yl)-2-oxyethyl)amino)-1-oxopropyl-2-yl)(methyl)tert-butyl carbamate (C-14-1) (117 mg, 0.20 mmol, 1.0 equivalent) in dichloromethane (0.5 mL) was added hydrochloric acid solution (2 mL, 4 mol/L in 1,4-dioxane), and stirred at room temperature for 3 h. The mixture was concentrated to obtain a crude product, which was further purified by reversed phase preparative liquid chromatography to obtain (S)-N-((S)-1-cyclohexyl-2-(6-(4-fluorobenzyl)-5-methoxy-2,3-dihydro-1H-pyrrolo[3,2-b]pyridine-1-yl)-2-oxyethyl)-2-(methylamino)propanamide (C-15-1) (62 mg, yield 60%).

¹H NMR (400 MHz, DMSO-d₆) δ 8.25 (s, 1H), 8.16 (s, 1H), 8.09 (d, J=9.0 Hz, 1H), 7.29-7.23 (m, 2H), 7.07 (t, J=8.7 Hz, 2H), 4.49 (d, J=9.0 Hz, 1H), 4.24 (t, J=8.3 Hz, 2H), 3.74-3.72 (m, 3H), 3.39 (s, 3H), 3.30-3.22 (m, 2H), 3.21-3.16 (m, 1H), 2.22 (s, 3H), 1.14 (d, J=6.6 Hz, 3H), 0.98 (s, 9H).

LCMS: ESI-MS(+), [M+1]=483.3.

### Synthesis of (S)-N-((S)-1-cyclohexyl-2-(6-(4-fluorobenzyl)-5-oxo-2,3,4,5-tetrahydro-1H-pyrrolo[3,2-b]pyridine-1-yl)-2-oxyethyl)-2-(methylamino)propanamide (I-4-1)

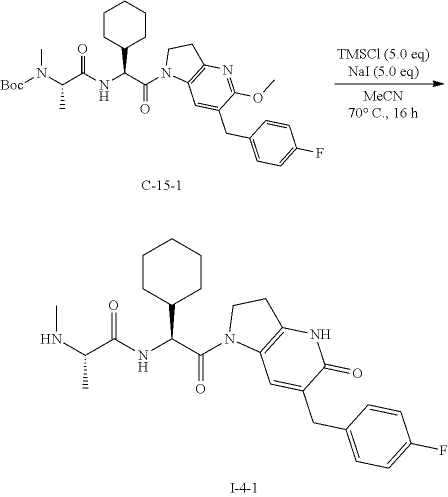 [SMILES: COc1nc2c(cc1Cc1ccc(F)cc1)N(C(=O)[C@@H](NC(=O)[C@H](C)N(C)C(=O)OC(C)(C)C)C1CCCCC1)CC2 ; CN[C@@H](C)C(=O)N[C@H](C(=O)N1CCc2[nH]c(=O)c(Cc3ccc(F)cc3)cc21)C1CCCCC1]

To a solution of ((S)-1-(((S)-1-cyclohexyl-2-(6-(4-fluorobenzyl)-5-methoxy-2,3-dihydro-1H-pyrrolo[3,2-b]pyridine-1-yl)-2-oxyethyl)amino)-1-oxopropyl-2-yl)(methyl)tert-butyl carbamate (C-15-1) (34 mg, 0.058 mmol, 1.0 equivalent) in acetonitrile (2 mL) was added sodium iodide (43 mg, 0.29 mmol, 5.0 equivalents) and trimethylchlorosilane (32.4 mg, 0.29 mmol, 5.0 equivalents) slowly at room temperature, and stirred for 2 h. The mixture was then heated to 70° C. and stirred overnight. The mixture was concentrated to obtain the crude product, which was further purified by reversed phase preparative liquid chromatography to obtain (S)-N-((S)-1-cyclohexyl-2-(6-(4-fluorobenzyl)-5-oxo-2,3,4,5-tetrahydro-1H-pyrrolo[3,2-b]pyridine-1-yl)-2-oxyethyl)-2-(methylamino)propanamide (I-4-1) (8 mg, yield 30%).

¹H NMR (400 MHz, DMSO-d₆) δ 8.25 (s, 1H), 8.14 (s, 1H), 8.05 (d, J=8.5 Hz, 1H), 7.21 (dd, J=8.4, 5.7 Hz, 2H), 7.09 (t, J=8.9 Hz, 2H), 4.46 (t, J=8.1 Hz, 1H), 4.31 (t, J=8.8 Hz, 1H), 4.22 (q, J=10.0, 9.5 Hz, 1H), 3.84 (s, 5H), 3.17 (t, J=8.6 Hz, 2H), 3.01 (q, J=6.8 Hz, 1H), 2.17 (s, 3H), 1.77-1.52 (m, 6H), 1.24-0.98 (m, 8H).

LCMS: ESI-MS(+), [M+1]=469.3.

The following compounds were prepared by referring to Embodiment 59:

| Embodiment | No. | ¹H-NMR | MS |
|---|---|---|---|
| Embodiment | I-4-2 | ¹H NMR (400 MHz, Methanol-d₄) δ 8.51 | ESI-MS: |
| 60 | | (s, 1H), 8.22 (s, 1H), 7.30 (q, J = 8.0 Hz, | [M + H]⁺ = 461.30 |
| | | 1H), 6.90 (dd, J = 11.8, 8.9 Hz, 2H), 4.59 | |
| | | (s, 1H), 4.50-4.41 (m, 1H), 4.33-4.25 (m, | |
| | | 1H), 3.81 (s, 2H), 3.72 (q, J = 7.2 Hz, 1H), | |
| | | 3.20 (q, J = 8.4, 6.8 Hz, 2H), 2.55 (s, 3H), | |
| | | 1.39 (d, J = 6.7 Hz, 3H), 1.08 (s, 9H). | |
| Embodiment | I-4-3 | ¹H NMR (400 MHz, DMSO-d₆) δ 8.40 (d, | ESI-MS: 443.3 |
| 61 | | J = 8.8 Hz, 1H), 8.09 (s, 1H), 7.28-7.21 | [M + H]⁺ |
| | | (m, 2H), 7.12-7.05 (m, 2H), 4.52 (d, J = | |
| | | 8.7 Hz, 1H), 4.20 (ddd, J = 12.9, 9.9, 7.8 | |
| | | Hz, 2H), 3.73 (d, J = 9.3 Hz, 2H), 3.55 (d, | |
| | | J = 7.0 Hz, 1H), 3.07 (t, J = 8.9 Hz, 2H), | |
| | | 2.37 (s, 3H), 1.22 (d, J = 6.8 Hz, 3H), 0.99 | |
| | | (s, 9H). | |
| Embodiment | I-4-4 | ¹H NMR (400 MHz, DMSO-d₆) δ 8.92- | ESI-MS: 489.3 |
| 62 | | 8.76 (m, 2H), 8.73 (d, J = 8.4 Hz, 1H), | [M + H]⁺ |
| | | 8.02 (s, 1H), 7.33 (td, J = 8.7, 6.7 Hz, 1H), | |
| | | 7.20 (td, J = 9.9, 2.6 Hz, 1H), 7.03 (td, J = | |
| | | 8.5, 2.5 Hz, 1H), 4.52 (d, J = 8.4 Hz, 1H), | |
| | | 4.06 (d, J = 10.5 Hz, 2H), 3.91 (d, J = 7.4 | |
| | | Hz, 2H), 3.74 (d, J = 5.4 Hz, 3H), 1.29 (t, | |
| | | J = 6.7 Hz, 9H), 1.01 (s, 9H). | |

### Embodiment 63: Synthesis of (S)-N-((S)-3,3-dimethyl-1-oxo-1-(5-oxo-6-phenyl-2,3,5,6-tetrahydro-1H-pyrrolo[2,3-c]pyridine-1-yl)propane-2-yl)-2-(methylamino)propanamide (I-5-1)

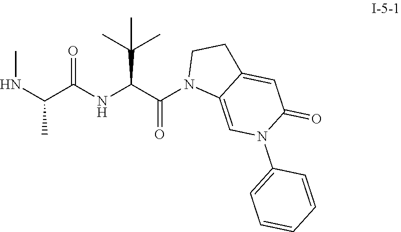 [SMILES: CN[C@@H](C)C(=O)N[C@H](C(=O)N1CCc2cc(=O)n(-c3ccccc3)cc21)C(C)(C)C]

### Synthesis of 5-oxo-2,3,5,6-tetrahydro-1H-pyrrolo[2,3-c]pyridine-1-tert-butyl formate (D-1)

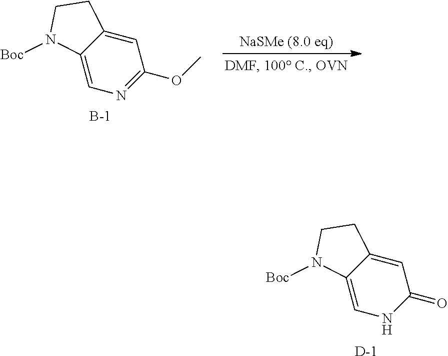

To a solution of B-1 (500 mg, 2.00 mmol, 1.0 equivalent) in N,N-dimethylformamide (5 mL) was added sodium methylmercaptan (1.12 g, 15.98 mmol, 8.0 equivalents). After addition, the mixture was heated to 100° C. and stirred for 16 h. The reaction mixture was concentrated under vacuum to remove N,N-dimethylformamide, and stirred in dichloromethane for 0.5 h. The reaction mixture was filtered and concentrated the filtrate to give the crude production, which was then purified by silica gel column chromatography to obtain D-1 (200 mg, yield 42.4%).

LCMS: ESI-MS(+): 271.34 [M+1]

### Synthesis of (5-oxo-6-phenyl-2,3,5,6-tetrahydro-1H-pyrrolo[2,3-c]pyridine-1-tert-butyl formate (D-2-1)

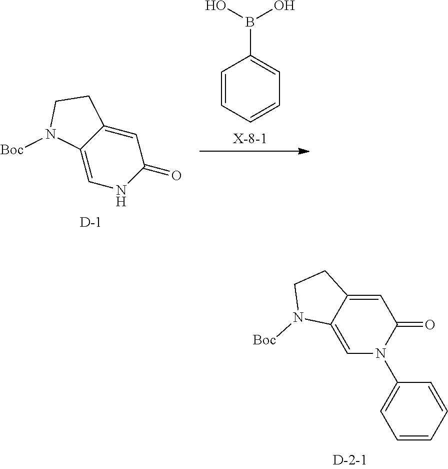

A suspension of D-1 (100 mg, 0.424 mmol, 1.0 equivalent), phenylboronic acid (62 mg, 0.508 mol, 1.2 equivalents), cupric acetate (22 mg, 0.106 mmol, 0.25 equivalent), triethylamine (86 mg, 0.848 mmol, 2.0 equivalents) and pyridine (34 mg, 0.424 mmol, 1.0 equivalent) in dichloromethane (5 mL) was stirred at room temperature under oxygen for 4 h. The reaction mixture was added dichloromethane (10 mL) and washed with water (10 mL), ammonium chloride aqueous solution (10%, 10 mL) and brine (10 mL). The organic phase was dried (anhydrous sodium sulfate), and concentrated under vacuum to afford the crude product, which was purified by silica gel column chromatography (petroleum ether:ethyl acetate=1:1) to obtain D-2-1 (140 mg, yield 99%).

LCMS: ESI-MS(+) 625.42 [2M+1]

### Synthesis of 6-phenyl-1,2,3,6-tetrahydro-5H-pyrrolo[2,3-c]pyridine-5-one (D-3-1)

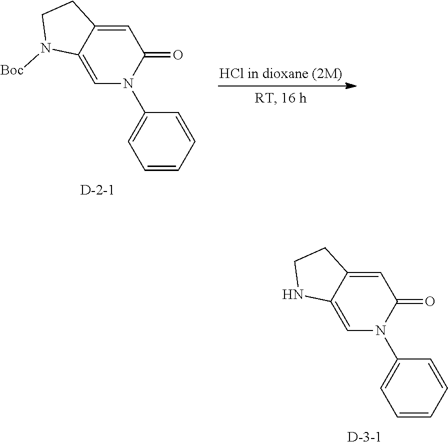 [SMILES: CC(C)(C)OC(=O)N1CCc2cc(=O)n(-c3ccccc3)cc21 ; O=c1cc2c(cn1-c1ccccc1)NCC2]

To a solution of D-2-1 (130 mg, 0.42 mmol, 1.0 equivalent) in 1,4-dioxane (1 mL) was added hydrogen chloride solution of (2 mol/L in 1,4-dioxane, 1 mL), and stirred at room temperature for 16 h. The reaction solution was concentrated under vacuum to afford the crude product, which was directly used at the next step.

LCMS: ESI-MS(+) 212.46 [M+1]

### Synthesis of (S)-(3,3-dimethyl-1-oxo-1-(5-oxo-6-phenyl-2,3,5,6-tetrahydro-1H-pyrrolo[2,3-c]pyridine)-1-yl)butane-2-yl)tert-butyl carbamate (D-4-1)

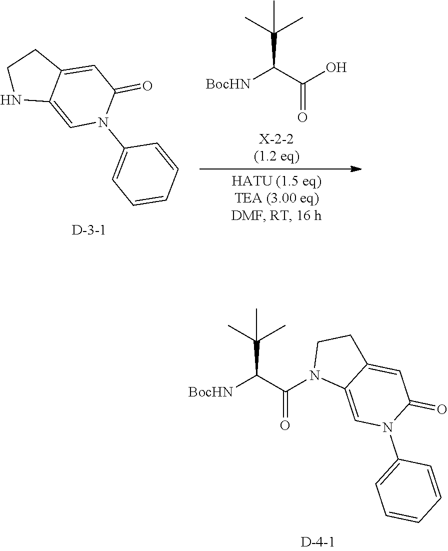 [SMILES: O=c1cc2c(cn1-c1ccccc1)NCC2 ; CC(C)(C)OC(=O)N[C@H](C(=O)O)C(C)(C)C ; CC(C)(C)OC(=O)N[C@H](C(=O)N1CCc2cc(=O)n(-c3ccccc3)cc21)C(C)(C)C]

To a solution of X-2-2 (82 mg, 0.36 mmol, 1.5 equivalents) and triethylamine (90 mg, 0.89 mmol, 3.0 equivalents) in anhydrous N,N-dimethylformamide (2 mL) was added 2-(7-azabenzotriazol-1-yl)-N,N,N′,N′-tetramethyluronium hexafluorophosphate (90 mL, 0.89 mmol, 3.0 equivalents), and stirred at 0° C. for 0.5 h. The reaction mixture was then added D-3-1 (63 mg, 0.30 mmol, 1.0 equivalent), and stirred at room temperature for 16 h. The reaction mixture was diluted with water (5 mL) and extracted with ethyl acetate (5 mL*3). The organic layer was washed with brine (5 mL*2), dried over anhydrous sodium sulfate and then concentrated under reduced pressure. The crude product was further purified by silica gel column chromatography (dichloromethane:methanol=20:1) to obtain D-4-1 (80 mg, yield 63%).

LCMS: ESI-MS(+), [M+1]=425.88

### Synthesis of (S)-1-(2-amino-3,3-dimethylbutyryl)-6-phenyl-1,2,3,6-tetrahydro-5H-pyrrolo[2,3-c]pyridine-5-one (D-5-1)

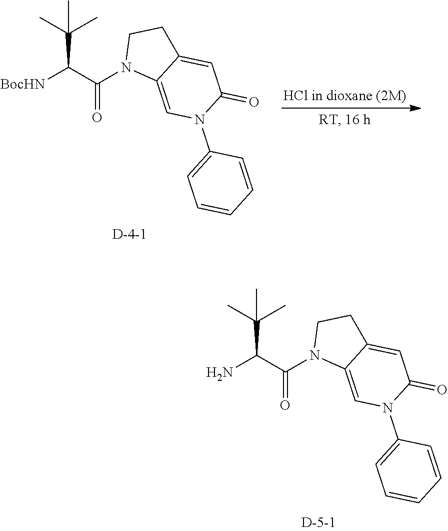 [SMILES: CC(C)(C)OC(=O)N[C@H](C(=O)N1CCc2cc(=O)n(-c3ccccc3)cc21)C(C)(C)C ; CC(C)(C)[C@H](N)C(=O)N1CCc2cc(=O)n(-c3ccccc3)cc21]

To a solution of D-4-1 (80 mg, 0.19 mmol, 1.0 equivalent) in 1,4-dioxane (1 mL) was added hydrogen chloride solution (4.0 mol/L in 1,4-dioxane, 1.0 mL), and stirred at room temperature for 16 h. The reaction was concentrated to give the crude product, which was directly used at the next step.

### Synthesis of ((S)-1-(((S)-3,3-dimethyl-1-oxo-1-(5-oxo-6-phenyl-2,3,5,6-tetrahydro-1H-pyrrole) [2,3-c]pyridine-1-yl)butane-2-yl)amino)-1-oxopropyl-2-yl)(methyl)tert-butyl carbamate (D-6-1)

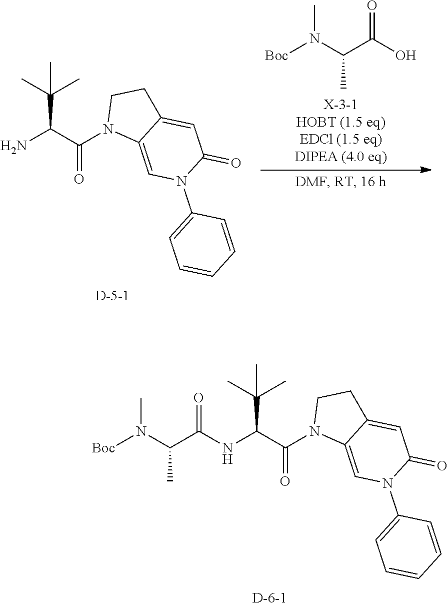 [SMILES: CC(C)(C)[C@H](N)C(=O)N1CCc2cc(=O)n(-c3ccccc3)cc21 ; C[C@@H](C(=O)O)N(C)C(=O)OC(C)(C)C ; C[C@@H](C(=O)N[C@H](C(=O)N1CCc2cc(=O)n(-c3ccccc3)cc21)C(C)(C)C)N(C)C(=O)OC(C)(C)C]

A suspension of D-5-1 (53 mg, 0.16 mmol, 1.0 equivalent), X-3-1 (44 mg, 0.21 mmol, 1.3 equivalents), 1-hydroxybenzotriazole (HOBT, 33 mg, 0.25 mmol, 1.5 equivalents), 1-(3-dimethylaminopropyl)-3-ethyl-carbodiimid monohydrochloride (47 mg, 0.25 mmol, 1.5 equivalents) and N,N-diisopropylethylamine (64 mg, 0.49 mmol, 4.0 equivalents) in anhydrous N,N-dimethylformamide (1 mL) was stirring at room temperature for 16 h. The reaction mixture was diluted with water (10 mL) and extracted with ethyl acetate (10 mL*3). The organic layer was washed with water (10 mL) and brine (10 mL), dried over anhydrous sodium sulfate and then concentrated under reduced pressure. The crude product was further purified by silica gel column chromatography (dichloromethane:methanol=20:1) to obtain D-6-1 (18 mg, yield 21.7%).

LCMS: ESI-MS (+) 511.1 [M+1]

### Synthesis of (S)-N-((S)-3,3-dimethyl-1-oxo-1-(5-oxo-6-phenyl-2,3,5,6-tetrahydro-1H-pyrrolo[2,3-c]pyridine-1-yl)butane-2-yl)-2-(methylamino)propanamide (I-5-1)

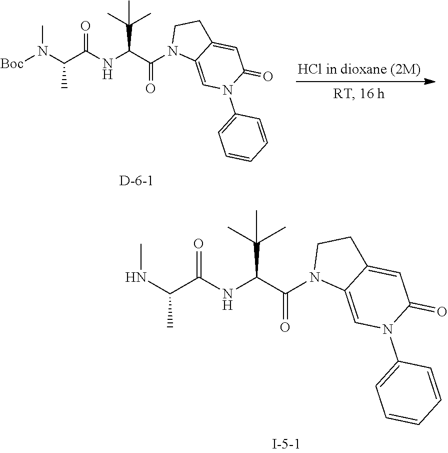 [SMILES: C[C@@H](C(=O)N[C@H](C(=O)N1CCc2cc(=O)n(-c3ccccc3)cc21)C(C)(C)C)N(C)C(=O)OC(C)(C)C ; CN[C@@H](C)C(=O)N[C@H](C(=O)N1CCc2cc(=O)n(-c3ccccc3)cc21)C(C)(C)C]

To a solution of D-6-1 (18 mg, 0.035 mmol, 1.0 equivalent) in 1,4-dioxane (1 mL) was added hydrogen chloride solution (4.0 mol/L in 1,4-dioxane, 1 mL) and stirring at room temperature for 16 h. The reaction solution was concentrated and purified by preparative liquid chromatography to obtain I-5-1 (15 mg, yield 99%).

¹H NMR (400 MHz, DMSO-d₆) δ 9.16 (s, 1H), 8.84 (s, 1H), 8.64 (d, J=8.1 Hz, 1H), 8.16 (s, 1H), 7.54 (t, J=7.5 Hz, 2H), 7.46 (t, J=7.3 Hz, 1H), 7.40 (d, J=7.7 Hz, 2H), 6.45 (s, 1H), 4.59 (d, J=8.1 Hz, 1H), 4.35-4.21 (m, 2H), 3.99 (d, J=7.4 Hz, 1H), 3.19 (s, 2H), 1.37 (d, J=6.9 Hz, 3H), 1.06 (s, 9H).

LCMS: ESI-MS(+) 411.20 [M+1]

The following compounds were prepared by referring to the preparation method in Embodiment 63:

| Embodi- | | | |
|---|---|---|---|
| ment | No. | ¹H-NMR | MS |
| Embodi- | I-5-2 | ¹H NMR (400 MHz, DMSO-d₆) δ 9.51 (s, | ESI-MS: |
| ment | | 1H), 8.29 (d, J = 8.5 Hz, 1H), 8.23 (s, | 468.4 |
| 64 | | 1H), 8.13 (d, J = 2.0 Hz, 2H), 7.51 (dd, | [M + H]⁺ |
| | | J = 8.6, 2.0 Hz, 1H), 6.47 (s, 1H), 4.56 (s, | |
| | | 1H), 4.29 (s, 2H), 3.19 (d, J = 7.6 Hz, | |
| | | 3H), 2.23 (s, 2H), 1.15 (s, 3H), 0.99 (s, | |
| | | 9H). | |

By referring to the method described in the Description of WO2008/128171A2, SM-406 was prepared (the compound in Embodiment 16 of the Description of WO2008/128171A2).

| No. | ¹H-NMR | MS |
|---|---|---|
| SM- | ¹H NMR (400 MHz, DMSO-d₆) δ 8.94 (d, J = 8.5 | ESI-MS: |
| 406 | Hz, 1H), 8.19 (d, J = 6.4 Hz, 1H), 7.24-7.38 (m, | 562.3[M + |
| | 10H), 6.10 (d, J = 8.4 Hz, 1H), 4.51-4.60 (m, 2H), | H]⁺ |
| | 4.06-4.12 (m, 1H), 3.78-3.84 (m, 2H), 3.10- | |
| | 3.15 (m, 1H), 2.90-2.98 (m, 2H), 1.46-2.39 (m, | |
| | 1H), 2.15-2.35 (m, 5H), 2.12 (s, 1H), 1.91-2.06 | |
| | (m, 3H), 1.78-1.85 (m, 1H), 1.63-1.67 (m, 1H), | |
| | 1.51-1.58 (m, 1H), 1.14 (d, J = 6.9 Hz, 3H), | |
| | 0.88-0.93 (m, 6H). | |

### Embodiment 65: Binding of Inhibitors to IAP Proteins

The binding affinity of the compounds provided by the present invention to IAP proteins was determined using a fluorescence polarization method (Nikolovska-Colesak et al, Anal. Biochem. 2004, 332:261-73). The recombinant BIR3 domains of human XIAP (residues 238-358), human cIAP1 (residues 255-364) and human cIAP2 (residues 236-342) fusing GST-tags were expressed in E. coli and purified using glutathione agarose 4B affinity chromatography and gel filtration chromatography. A 5-Fam fluorescently modified peptide probe (AbuRPFK(5-Fam)-NH2) was used to test the competitive binding ability of the compounds to the BIR3 domains of XIAP, cIAP1 and cIAP2. The peptide probe (5 nM), XIAP BIR3 (30 nM) or cIAP1 BIR3 (5 nM) or cIAP2 BIR3 (10 nM), and serial diluted compounds were mixed in a test buffer (100 mM potassium phosphate pH 7.5, 100 μg/ml bovine gamma globulin, 0.02% sodium azide, and 1 mM DTT). After incubating the samples at room temperature for 1 hour, the fluorescence polarization values were read using a Tecan microplate reader (FP excitation wavelength 485 nm, absorption wavelength 530 nm). The IC₅₀ value was obtained by fitting the curve using nonlinear least square regression with the Graphpad Prism software. The inhibition constant Kᵢ, values were calculated using the Equation Kᵢ, [I]₅₀/([L]₅₀/K_{d}+[P]₀/Kd+1).

In the binding assay, as shown in Table 1, the compounds provided by the present invention exhibited strong binding to the BIR3 domains of XIAP, cIAP1 and cIAP2. Specifically, Embodiments 18, 25, 29, 34, 36-37, 40-42, 47-52, 55-57, 59-62 showed a stronger binding activity to the cIAP1 BIR3 domain compared to compound SM-406 from WO2008/128171. Embodiments 18, 25, 29, 34, 36-37, 41, 48-52, 55-57, 59-62 demonstrated a stronger binding activity to the cIAP2 BIR3 domain compared to compound SM-406 from WO2008/128171. Additionally, Embodiment 34, 36, 48, 51-52, 55, 57, 59-61 showed a stronger binding activity to the XIAP BIR3 domain compared to compound SM-406 from WO2008/128171.

TABLE 1

| | | cIAP1 BIR3 | cIAP2 BIR3 | XIAP BIR3 |
|---|---|---|---|---|
| Embodiment | No. | Ki (nM) | Ki (nM) | Ki (nM) |
| 18 | I-2-4 | 1.3 | 3.2 | 73.2 |
| 25 | I-2-11 | 0.7 | 1.7 | 31.4 |
| 29 | I-2-15 | 1.0 | 4.0 | 76.9 |
| 34 | I-2-20 | 1.0 | 1.8 | 21.5 |
| 36 | I-2-22 | 0.8 | 1.5 | 21.0 |
| 37 | I-2-23 | 1.0 | 2.3 | 53.6 |
| 40 | I-2-26 | 1.4 | 6.9 | 70.9 |
| 41 | I-2-27 | 1.9 | 5.7 | 208.3 |
| 42 | I-2-28 | 1.9 | 6.7 | 184.8 |
| 43 | I-2-29 | 3.0 | 12.7 | 567.5 |
| 46 | I-2-32 | 2.2 | 10.0 | 342.8 |
| 47 | I-2-33 | 1.9 | 7.9 | 287.8 |
| 48 | I-2-34 | 1.1 | 1.5 | 24.1 |
| 49 | I-2-35 | 1.2 | 2.6 | 33.4 |
| 50 | I-3-1 | 1.2 | 2.6 | 30.7 |
| 51 | I-3-2 | 1.2 | 2.2 | 18.4 |
| 52 | I-3-3 | 1.4 | 2.2 | 13.7 |
| 55 | I-3-6 | 0.8 | 1.6 | 12.3 |
| 56 | I-3-7 | 1.6 | 5.2 | 45.2 |
| 57 | I-3-8 | 1.3 | 2.1 | 7.1 |
| 59 | I-4-1 | 1.3 | 2.7 | 19.9 |
| 60 | I-4-2 | 1.3 | 2.4 | 9.4 |
| 61 | I-4-3 | 1.0 | 1.9 | 8.2 |
| 62 | I-4-3 | 1.6 | 3.8 | 55.2 |

| | | | |
|---|---|---|---|
| Compound SM-406 | 2.3 | 5.8 | 25.2 |
| from Embodiment 16 | | | |
| of WO2008/128171 | | | |

### Embodiment 66: Effects of Inhibitors on Proliferation of Different Cancer Cell Lines

The effects of the compounds provided by the present invention on the proliferation of different cancer cell lines were tested using the WST-8 assay kit. This method is based on the reduction of WST-8 by dehydrogenase in live cells to form a highly water-soluble yellow Formazan dye. Cells were seeded in a 96-well plate and cultured with the test compounds at 37° C. under 5% CO₂ for 72 hours. Afterwards, 10% (v/v) WST-8 was added, and the samples were incubated for an additional 2-3 hours. The absorbance of the samples was measured at 450 nm using a Tecan microplate reader, and the concentration of the compound that inhibited 50% of cell proliferation (IC₅₀, nM) was calculated. The activity of the compounds provided by the present invention was determined in three cancer cell lines: MDA-MB-231 human breast cancer cells, SK-OV-3 human ovarian cancer cells, and PANC1 human pancreatic cancer.

The IC₅₀ values of the test compounds provided by the present invention against three cancer cell lines were all lower than those of Embodiment 16 (compound SM-406) from WO2008/128171. This indicates that the compounds provided by the present invention are potent inhibitors of cancer cell growth and have stronger inhibitory activity compared to compound SM-406 from Embodiment 16 of WO2008/128171. The IC₅₀ data of the compounds of this invention are provided in Table 2.

TABLE 2

| | | MDA-MB-231 | SKOV-3 | PANC1 |
|---|---|---|---|---|
| Embodiment | No. | IC₅₀ (nM) | IC₅₀ (nM) | IC₅₀ (nM) |
| 18 | I-2-4 | 8 | 13 | 75 |
| 25 | I-2-11 | 5 | 4 | 38 |
| 29 | I-2-15 | 9 | 12 | 64 |
| 34 | I-2-20 | 3 | 1 | 33 |
| 36 | I-2-22 | 3 | 2 | 34 |
| 37 | I-2-23 | 9 | 5 | 67 |
| 40 | I-2-26 | 21 | 7 | 89 |
| 41 | I-2-27 | 18 | 19 | 96 |
| 42 | I-2-28 | 20 | 23 | 124 |
| 43 | I-2-29 | 29 | 32 | 165 |
| 46 | I-2-32 | 28 | 29 | 189 |
| 47 | I-2-33 | 24 | 27 | 192 |
| 50 | I-3-1 | 5 | 4 | 58 |
| 51 | I-3-2 | 2 | 2 | 43 |
| 52 | I-3-3 | 2 | 1 | 37 |
| 59 | I-4-1 | 6 | 1 | 53 |
| 60 | I-4-2 | 4 | 1 | 16 |

| | | | |
|---|---|---|---|
| Compound SM-406 | 33 | 40 | 241 |
| from Embodiment 16 | | | |
| of WO2008/128171 | | | |

### Embodiment 67: Inhibitor-Induced Apoptosis in MDA-MB-231 Cancer Cells

The effect of the compounds provided by the present invention and compound SM-406 from WO2008/128171 on inducing apoptosis was tested in MDA-MB-231 cancer cells. After treating the cells with 10 nM and 100 nM concentrations of the compounds for 3 hours, the induction of apoptosis was analyzed using flow cytometry with Annexin V and propidium iodide staining. The proportions (%) of apoptotic cells relative to the total number of cells are provided in Table 3. The compounds provided by the present invention induced strong apoptotic cell deaths at both 10 nM and 100 nM concentrations, with significantly stronger activity compared to compound SM-406 from Embodiment 16 of WO2008/128171. The results demonstrate that the compounds of this invention strongly induce apoptotic cell death.

TABLE 3

| | | Proportion of apoptotic cells (%) | |
|---|---|---|---|
| | | Drug concentration | Drug concentration |
| Embodiment | No. | 10 nM | 100 nM |
| 18 | I-2-4 | 32 | 45 |
| 25 | I-2-11 | 35 | 48 |
| 29 | I-2-20 | 31 | 47 |
| 34 | I-2-20 | 39 | 53 |
| 36 | I-2-22 | 38 | 52 |
| 37 | I-2-23 | 35 | 50 |
| 40 | I-2-26 | 32 | 47 |
| 41 | I-2-27 | 31 | 46 |
| 42 | I-2-28 | 29 | 47 |
| 43 | I-2-29 | 20 | 42 |
| 46 | I-2-32 | 21 | 43 |
| 47 | I-2-33 | 23 | 45 |
| 50 | I-3-1 | 36 | 48 |
| 51 | I-3-2 | 38 | 52 |
| 52 | I-3-3 | 43 | 55 |
| 59 | I-4-1 | 36 | 49 |
| 60 | I-4-2 | 41 | 54 |

| | | |
|---|---|---|
| Compound SM-406 | 6 | 34 |
| from Embodiment 16 | | |
| of WO2008/128171 | | |

1.-12. (canceled)

13. A pyridone compound, with a structure as shown in Formula (I), comprising its isomer, prodrug, stable isotope derivative, pharmaceutically acceptable salt or a mixture thereof: 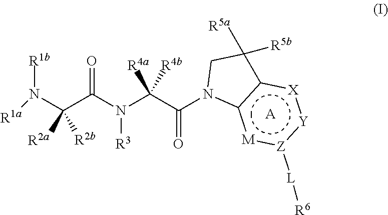
where:
R^{1a} or R^{1b} is independently selected from hydrogen or C₁˜C₆ alkyl or halogenated C₁˜C₆ alkyl, or R^{1a} or R^{1b} and the connected nitrogen atom together forming 3-7-membered azacyclic alkyl;
R^{2a} or R^{2b} is independently selected from hydrogen, C₁˜C₆ alkyl or halogenated C₁˜C₆ alkyl, or R^{2a} or R^{2b} and the connected carbon atom together forming C₃˜C₆ cycloalkyl or C₃˜C₆ heterocyclic alkyl, and when R^{2a} and R^{2b} are different, the carbon atom connected to R^{2a} and R^{2b} being of racemic configuration, R configuration or S configuration;
R³ is selected from hydrogen, C₁˜C₆ alkyl or C₅˜C₁₂ aryl;
R^{4a} or R^{4b} is independently selected from hydrogen, substituted C₁˜C₆ alkyl, substituted C₅˜C₁₂ aryl, substituted C₃˜C₆ cycloalkyl or C₃˜C₆ heterocyclic alkyl, the substituent being selected from hydrogen, halogen, cyano, halogenated C₁˜C₆ alkyl, C₁˜C₆ alkyl, C₁˜C₆ alkoxy, C₃˜C₆ cycloalkyl or heterocyclic alkyl, hydroxyl, amino, methylamino, dimethylamino, acetamino, carboxyl, methoxycarbonyl or nitro, there being one or more substituents, and when R^{4a} and R^{4b} are different, the connected carbon atom being of racemic configuration, R configuration or S configuration;
R^{5a} or R^{5b} is independently selected from hydrogen, C₁˜C₆ alkyl or R^{5a} or R^{5b} and the connected carbon atom together forming C₃˜C₆ cycloalkyl or C₃˜C₆ heterocyclic alkyl, and when R^{5a} and R^{5b} are different, the connected carbon atom being of racemic configuration, R configuration or S configuration;
R⁶ is selected from hydrogen, any substituted C₅˜C₁₂ aryl, any substituted C₃˜C₁₂ cycloalkyl or heterocyclic alkyl, the substituent being selected from hydrogen, halogen, cyano, halogenated C₁˜C₆ alkyl, C₁˜C₆ alkyl, C₁˜C₆ alkoxy, hydroxyl, amino, methylamino, dimethylamino, acetamino, carboxyl, methoxycarbonyl or nitro, and there being one or more substituents;
The ring A is a heteroaromatic ring, where when M is carbonyl, X and Y are CH and Z is N; when M is CH, Y is carbonyl, Z is C or N and X is CH or NR⁷;
R⁷ is selected from hydrogen, substituted C₁˜C₆ alkyl, substituted C₅˜C₁₂ aryl, substituted C₃˜C₆ cycloalkyl or C₃˜C₁₀ heterocyclic alkyl; the substituent being selected from hydrogen, halogen, cyano, halogenated C₁˜C₆ alkyl, C₁˜C₆ alkyl, C₁˜C₆ alkoxy, C₃˜C₆ cycloalkyl or C₃˜C₆ heterocyclic alkyl, hydroxyl, methoxy, amino, methylamino, dimethylamino, acetamino, carboxyl, methoxycarbonyl or nitro, and there is one or more substituents;
L is selected from oxygen, sulfur, 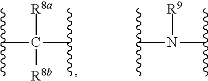
or —(CH₂)ₙ—, where the wavy lines represent points connecting the rest part of the compound;
R^{8a} or R^{8b} is independently selected from hydrogen, halogen, C₁˜C₆ alkyl or C₅˜C₁₂ aryl, and when R^{8a}, R^{8b} and R⁶ are different, the connected carbon atom is of racemic configuration, R configuration or S configuration;
R⁹ is selected from hydrogen or C₁˜C₆ alkyl or C₅˜C₁₂ aryl;
n ranging from 0 to 3.

14. The pyridone compound according to claim 13, wherein:
R^{1a} is selected from hydrogen, methyl or ethyl;
R^{1b} is hydrogen;
R^{2a} or R^{2b} is independently selected from hydrogen, methyl or ethyl, or R^{2a} or R^{2b} and the connected carbon atom together forming cyclopropyl, oxacyclobutyl and cyclobutyl, and when R^{2a} is hydrogen, R^{2b} being methyl or ethyl, and the carbon atom connected to R^{2b} is of racemic configuration, R configuration or S configuration;
R³ is selected from hydrogen, methyl or ethyl;
R^{4a} or R^{4b} is independently selected from hydrogen, cyclopropyl methyl, cyclopentyl, cyclohexyl, tetrahydropyranyl, isopropyl or tert-butyl and the carbon atom connected to R^{4a} and R^{4b} being of R or S configuration;
R^{5a} and R^{5b} are both hydrogen or methyl, or R^{5a} or R^{5b} and the connected carbon atom together form cyclopropyl, cyclopentyl or cyclohexyl;
R⁶ is selected from hydrogen, substituted C₅˜C₁₂ aryl, substituted C₅˜C₁₂ heteroaryl, substituted C₃˜C₆ cycloalkyl or C₃˜C₁₀ heterocyclic alkyl pyrimidyl, the substituent being selected from hydrogen, halogen, cyano, methoxy, hydroxyl or trifluoromethyl, and there is one or more substituents;
the ring A is a pyridine ring, when M is carbonyl, X and Y is CH and Z is N; when M is CH, Y is carbonyl, Z is C or N and X is CH or NR⁷;
R⁷ is selected from hydrogen, C₁˜C₆ alkyl or C₁˜C₆ cycloalkyl;
L is selected from 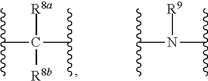
or —(CH₂)ₙ—, where the wavy lines represent points connecting the rest part of the compound;
R^{8a} or R^{8b} is independently selected from hydrogen, halogen, C₁˜C₆ alkyl or C₅˜C₁₂ aryl, and when R^{8a}, R^{8b} and R⁶ are different, the connected carbon atom is of racemic configuration;
R⁹ is selected from hydrogen or methyl;
n ranging from 0 to 3.

15. The pyridone compound according to claim 13, wherein
R^{1a} is selected from hydrogen or methyl;
R^{1b} is selected from hydrogen;
R^{2a} or R^{2b} is independently selected from hydrogen or methyl, or R^{2a} or R^{2b} and the connected carbon atom together forming cyclopropyl, oxacyclobutyl and cyclobutyl, and when R^{2a} is hydrogen, R^{2b} is methyl or ethyl, and the carbon atom connected to R^{2b} is S configuration;
R³ is selected from hydrogen;
R^{4a} or R^{4b} is independently selected from hydrogen, cyclopropyl methyl, cyclopentyl, cyclohexyl, tetrahydropyranyl, isopropyl or tert-butyl and the carbon atom connected to R^{4a} and R^{4b} being of R or S configuration;
R^{5a} and R^{5b} are both hydrogen or methyl;
R⁶ is selected from hydrogen, substituted phenyl, substituted pyridinyl or substituted pyrimidyl, the substituent being selected from hydrogen, halogen, cyano, methoxy, hydroxyl, hydroxyl or trifluoromethyl, and there is one or more substituents;
the ring A being a pyridine ring, where when M is carbonyl, X and Y is CH and Z is N; when M is CH, Y is carbonyl, Z is C or N and X is CH or NR⁷;
R⁷ is selected from hydrogen or methyl;
L is selected from 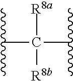
or —(CH₂)ₙ—, where the wavy lines represent points connecting the rest part of the compound;
R^{8a} is selected from hydrogen, halogen, methyl or phenyl, R^{8b} is hydrogen, and when R^{8a}, R^{8b} and R⁶ are different, the connected carbon atom is of racemic configuration;
n is selected from 0, 1 or 2.

16. The pyridone compound according to claim 13, which is a compound as shown in Formula (II-1), or tautomeric or stereochemically isomeric form thereof, a pharmaceutically acceptable salt or a solvate; where R^{1a}, R^{2a}, R^{2b}, R^{4a}, R^{4b}, R⁶ and L are as defined in any of claims 1 to 3

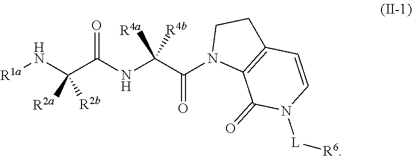

17. The pyridone compound according to claim 13, which is a compound as shown in Formula (II-2), or tautomeric or stereochemically isomeric form thereof, a pharmaceutically acceptable salt or a solvate; where R^{1a}, R^{2a}, R^{2b}, R^{4a}, R^{4b}, R^{5a}, R^{5b}, R⁶ and L are as defined in any of claims 1 to 3

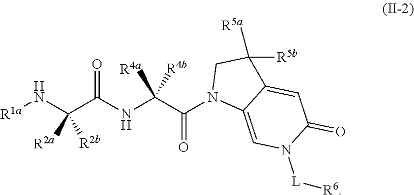

18. The pyridone compound according to claim 13, which is a compound as shown in Formula (II-3), or tautomeric or stereochemically isomeric form thereof, a pharmaceutically acceptable salt or a solvate; where R^{1a}, R^{2a}, R^{2b}, R^{4a}, R^{4b}, R⁶ and R⁷ are as defined in any of claims 1 to 3

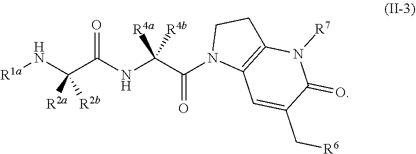

19. The pyridone compound according to claim 13, wherein in the structure:
R⁶ is selected from 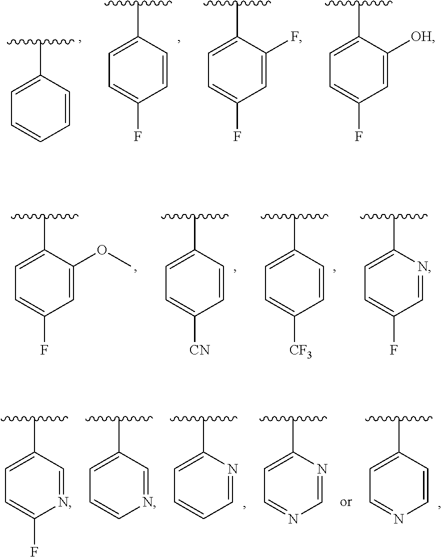
where the wavy lines represent points connecting the rest part of the compound.

20. The pyridone compound according to claim 13, wherein the pyridone compound is any of the following compounds:

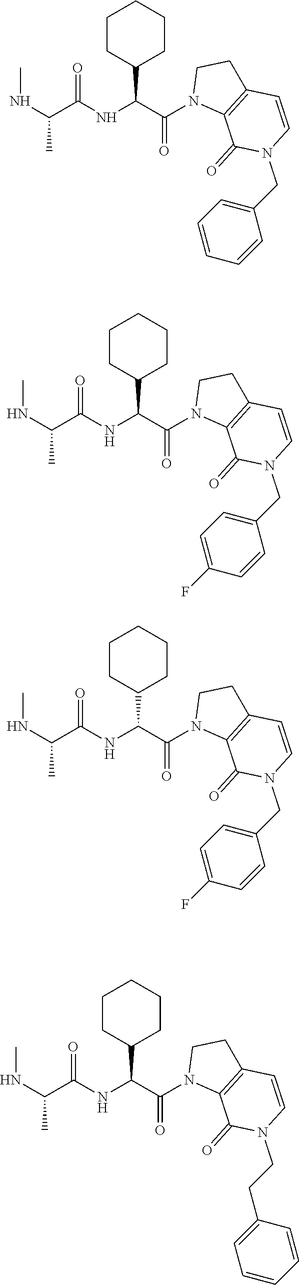 [SMILES: CN[C@@H](C)C(=O)N[C@H](C(=O)N1CCc2ccn(Cc3ccccc3)c(=O)c21)C1CCCCC1 ; CN[C@@H](C)C(=O)N[C@H](C(=O)N1CCc2ccn(Cc3ccc(F)cc3)c(=O)c21)C1CCCCC1 ; CN[C@@H](C)C(=O)N[C@@H](C(=O)N1CCc2ccn(Cc3ccc(F)cc3)c(=O)c21)C1CCCCC1 ; CN[C@@H](C)C(=O)N[C@H](C(=O)N1CCc2ccn(CCc3ccccc3)c(=O)c21)C1CCCCC1]

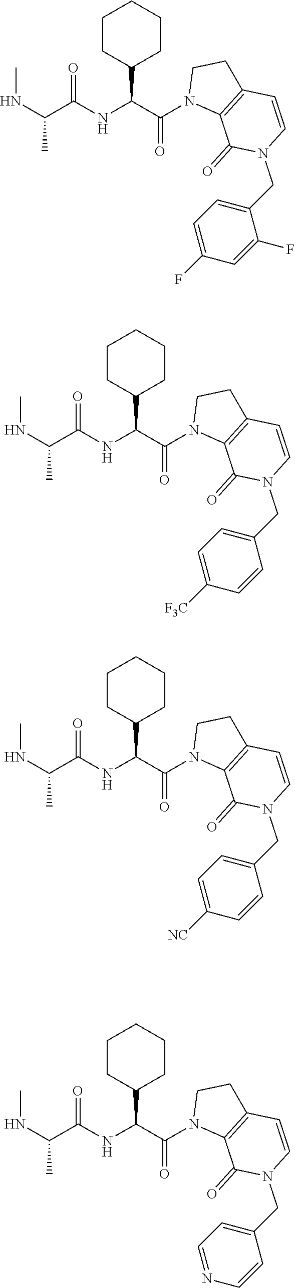 [SMILES: CN[C@@H](C)C(=O)N[C@H](C(=O)N1CCc2ccn(Cc3ccc(F)cc3F)c(=O)c21)C1CCCCC1 ; CN[C@@H](C)C(=O)N[C@H](C(=O)N1CCc2ccn(Cc3ccc(C(F)(F)F)cc3)c(=O)c21)C1CCCCC1 ; CN[C@@H](C)C(=O)N[C@H](C(=O)N1CCc2ccn(Cc3ccc(C#N)cc3)c(=O)c21)C1CCCCC1 ; CN[C@@H](C)C(=O)N[C@H](C(=O)N1CCc2ccn(Cc3ccncc3)c(=O)c21)C1CCCCC1]

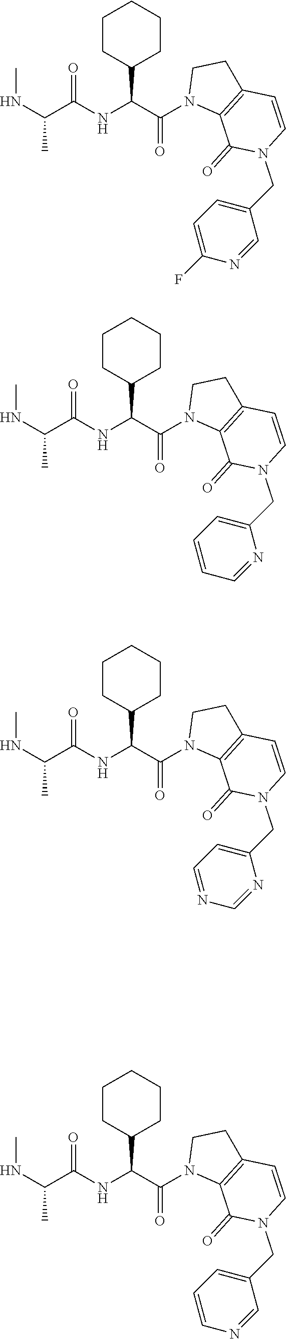 [SMILES: CN[C@@H](C)C(=O)N[C@H](C(=O)N1CCc2ccn(Cc3ccc(F)nc3)c(=O)c21)C1CCCCC1 ; CN[C@@H](C)C(=O)N[C@H](C(=O)N1CCc2ccn(Cc3ccccn3)c(=O)c21)C1CCCCC1 ; CN[C@@H](C)C(=O)N[C@H](C(=O)N1CCc2ccn(Cc3ccncn3)c(=O)c21)C1CCCCC1 ; CN[C@@H](C)C(=O)N[C@H](C(=O)N1CCc2ccn(Cc3cccnc3)c(=O)c21)C1CCCCC1]

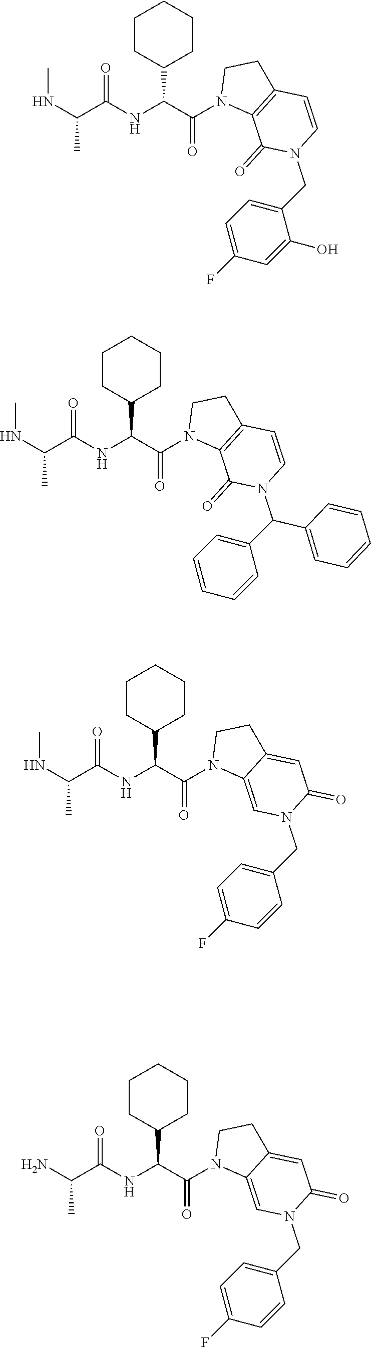 [SMILES: CN[C@@H](C)C(=O)N[C@@H](C(=O)N1CCc2ccn(Cc3ccc(F)cc3O)c(=O)c21)C1CCCCC1 ; CN[C@@H](C)C(=O)N[C@H](C(=O)N1CCc2ccn(C(c3ccccc3)c3ccccc3)c(=O)c21)C1CCCCC1 ; CN[C@@H](C)C(=O)N[C@H](C(=O)N1CCc2cc(=O)n(Cc3ccc(F)cc3)cc21)C1CCCCC1 ; C[C@H](N)C(=O)N[C@H](C(=O)N1CCc2cc(=O)n(Cc3ccc(F)cc3)cc21)C1CCCCC1]

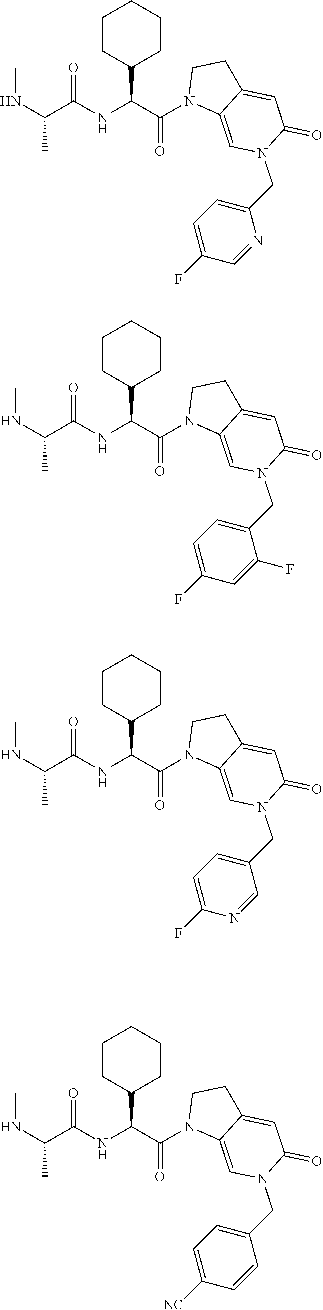 [SMILES: CN[C@@H](C)C(=O)N[C@H](C(=O)N1CCc2cc(=O)n(Cc3ccc(F)cn3)cc21)C1CCCCC1 ; CN[C@@H](C)C(=O)N[C@H](C(=O)N1CCc2cc(=O)n(Cc3ccc(F)cc3F)cc21)C1CCCCC1 ; CN[C@@H](C)C(=O)N[C@H](C(=O)N1CCc2cc(=O)n(Cc3ccc(F)nc3)cc21)C1CCCCC1 ; CN[C@@H](C)C(=O)N[C@H](C(=O)N1CCc2cc(=O)n(Cc3ccc(C#N)cc3)cc21)C1CCCCC1]

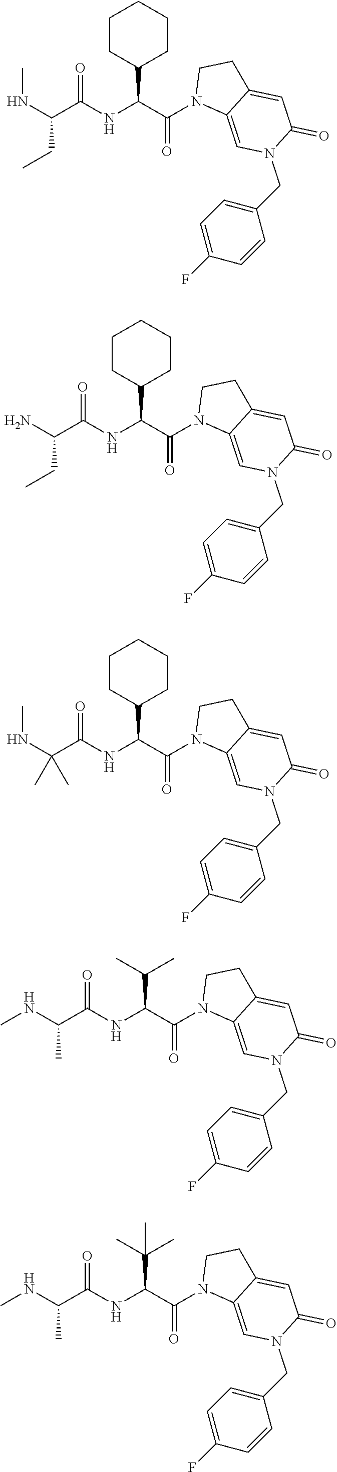

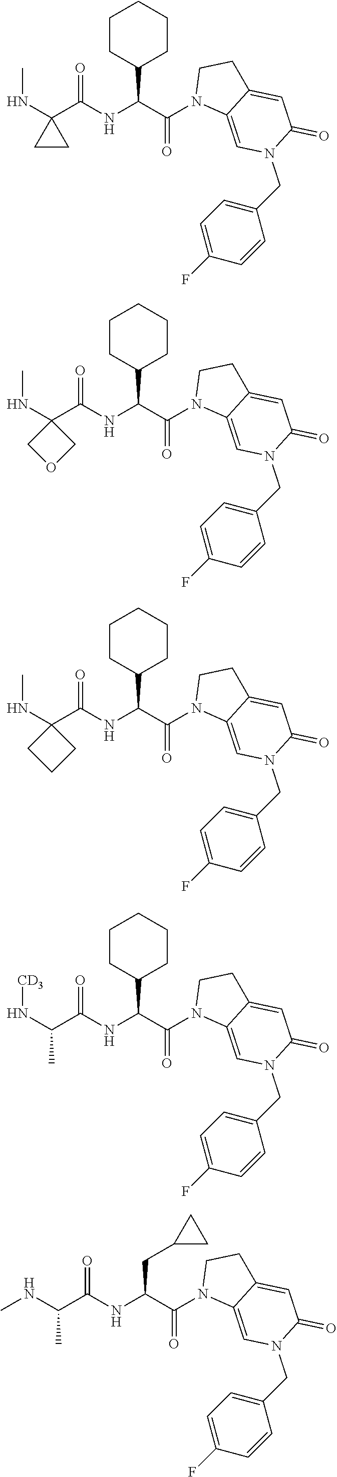

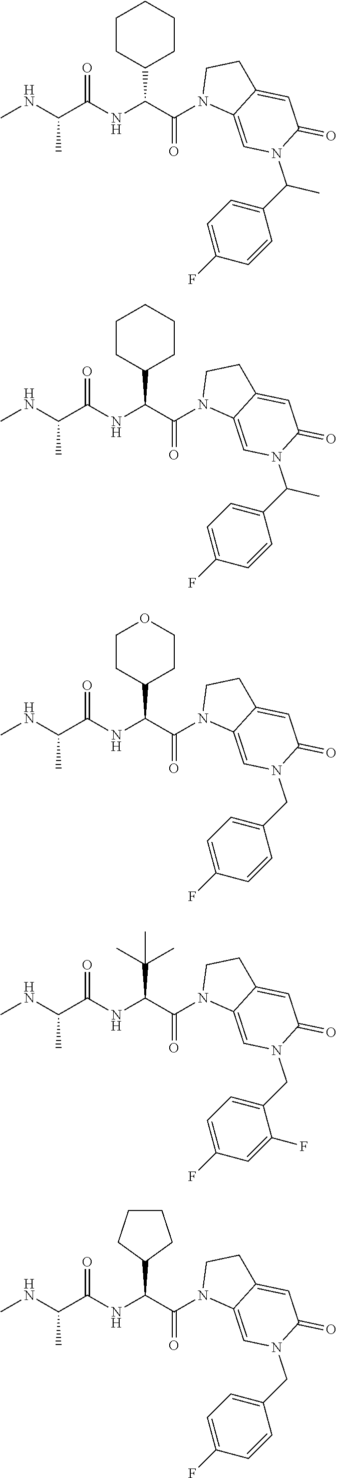

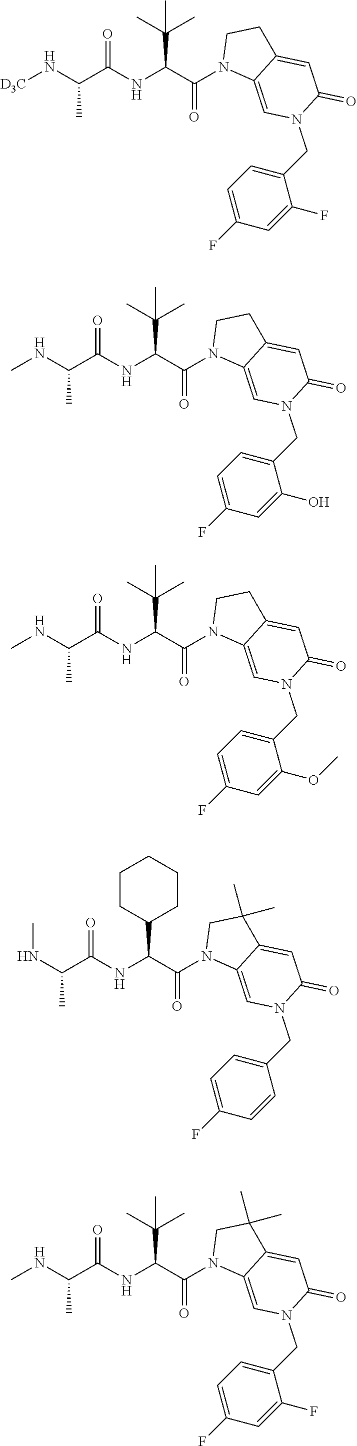 [SMILES: [2H]C([2H])([2H])N[C@@H](C)C(=O)N[C@H](C(=O)N1CCc2cc(=O)n(Cc3ccc(F)cc3F)cc21)C(C)(C)C ; CN[C@@H](C)C(=O)N[C@H](C(=O)N1CCc2cc(=O)n(Cc3ccc(F)cc3O)cc21)C(C)(C)C ; CN[C@@H](C)C(=O)N[C@H](C(=O)N1CCc2cc(=O)n(Cc3ccc(F)cc3OC)cc21)C(C)(C)C ; CN[C@@H](C)C(=O)N[C@H](C(=O)N1CC(C)(C)c2cc(=O)n(Cc3ccc(F)cc3)cc21)C1CCCCC1 ; CN[C@@H](C)C(=O)N[C@H](C(=O)N1CC(C)(C)c2cc(=O)n(Cc3ccc(F)cc3F)cc21)C(C)(C)C]

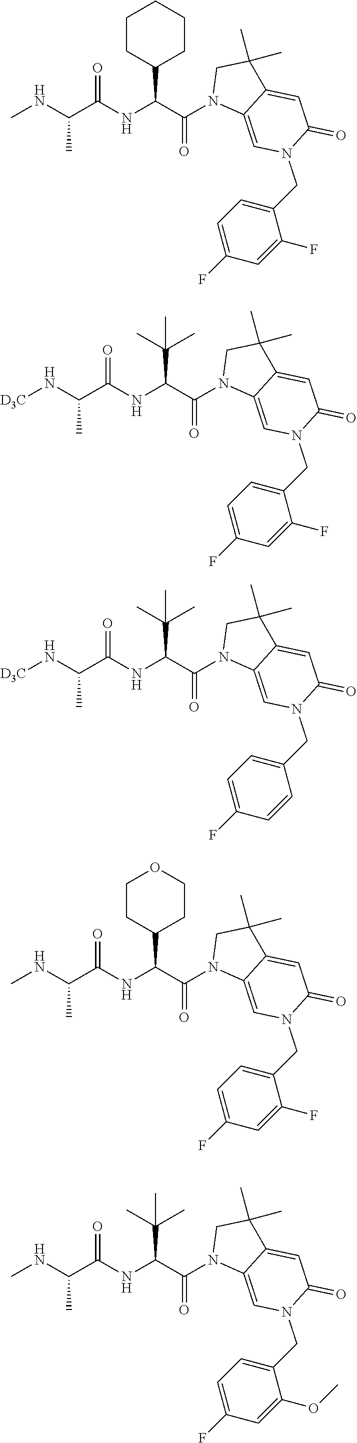

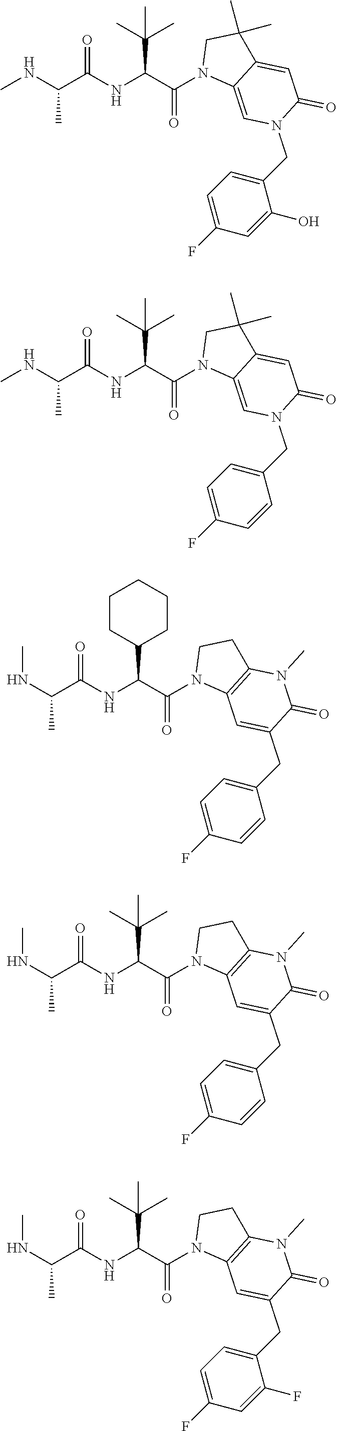 [SMILES: CN[C@@H](C)C(=O)N[C@H](C(=O)N1CC(C)(C)c2cc(=O)n(Cc3ccc(F)cc3O)cc21)C(C)(C)C ; CN[C@@H](C)C(=O)N[C@H](C(=O)N1CC(C)(C)c2cc(=O)n(Cc3ccc(F)cc3)cc21)C(C)(C)C ; CN[C@@H](C)C(=O)N[C@H](C(=O)N1CCc2c1cc(Cc1ccc(F)cc1)c(=O)n2C)C1CCCCC1 ; CN[C@@H](C)C(=O)N[C@H](C(=O)N1CCc2c1cc(Cc1ccc(F)cc1)c(=O)n2C)C(C)(C)C ; CN[C@@H](C)C(=O)N[C@H](C(=O)N1CCc2c1cc(Cc1ccc(F)cc1F)c(=O)n2C)C(C)(C)C]

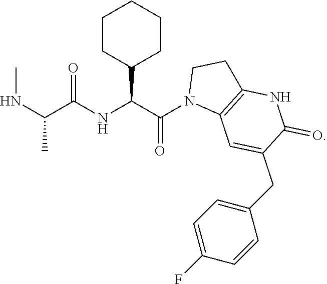 [SMILES: CN[C@@H](C)C(=O)N[C@H](C(=O)N1CCc2[nH]c(=O)c(Cc3ccc(F)cc3)cc21)C1CCCCC1]

21. The pyridone compound according to claim 13, wherein the pharmaceutically acceptable salt is a salt formed by the pyridone compound and an acid, and the acid being hydrochloric acid, hydrobromic acid, sulfuric acid, phosphoric acid, carbonic acid, methanesulfonic acid, benzenesulfonic acid, p-toluenesulfonic acid, naphthalene sulfonic acid, citric acid, tartaric acid, lactic acid, pyruvic acid, acetic acid, maleic acid, succinic acid, fumaric acid, salicylic acid, phenylacetic acid, mandelic acid or ferulic acid.

22. A pharmaceutical composition, comprising the pyridone compound in claim 13 and a pharmaceutically acceptable carrier.
